(19) United States
(12) Patent Application Publication
(10) Pub. No.: US 2010/0055702 A1
(43) Pub. Date: Mar. 4, 2010

(54) Pathogenecity Islands of Pseudomonas Aeruginosa

(75) Inventors: Scott E. Battle, Chicago, IL (US); Alan R. Hauser, Chicago, IL (US)

Correspondence Address: ANDRUS, SCEALES, STARKE & SAWALL, LLP, 100 EAST WISCONSIN AVENUE, SUITE 1100, MILWAUKEE, WI, 53202, US

(73) Assignee: Northwestern University, Evanston, IL (US)

(21) Appl. No.: 12/545,528

(22) Filed: Aug. 21, 2009

Related U.S. Application Data
(60) Provisional application No. 61/090,679, filed on Aug. 21, 2008.

(51) Int. Cl. C12Q 1/68; G01N 33/50

(52) U.S. Cl. 435/6; 436/94

(57) ABSTRACT

Disclosed are Pseudomonas aeruginosa Genomic Island nucleic acid sequences referred to as PAGI-5, PAGI-6, PAGI-7, PAGI-8, PAGI-9, PAGI-10, and PAGI-11. These nucleic acid sequences may be useful in methods for identifying virulent strains of Pseudomonas bacteria.

### CROSS-REFERENCE TO RELATED APPLICATIONS

The present application claims the benefit under 35 U.S.C. §119(e) to U.S. Provisional Application No. 61/090,679, filed on Aug. 21, 2008, the content of which is incorporated herein by reference in its entirety.

### STATEMENT REGARDING U.S. GOVERNMENT SPONSORED RESEARCH OR DEVELOPMENT

This invention was made with U.S. government support under grant Nos. K02 AI065615, F30-ES016487, and R01 AI075191 from the National Institutes of Health. The U.S. government has certain rights in this invention.

### BACKGROUND

The present invention relates generally to the field of Pseudomonas bacteria and methods for detecting virulent strains of Pseudomonas bacteria. In particular, the field relates to Pseudomonas aeruginosa and methods for detecting and assessing virulence strains thereof.

Pseudomonas aeruginosa is a medically important opportunistic pathogen that causes serious disease in hospitalized patients and individuals with cystic fibrosis (Fitzsimmons, 1993; Stryjewski et al., 2003). In the environment, it naturally inhabits lakes, streams, moist soil, and plant matter (Stryjewski, et al., 1974; Hoadley, 1977; Rhame, 1979) and has pathogenic activity against a wide spectrum of hosts, including mammals, worms, insects, fungi, amoebae, and plants (Alibaud et al., 2008; Glazebrook et al., 1978; Hogan et al., 2002; Jander et al., 2000; Mahajan-Miklos et al., 1999; Rahme et al., 1995).

Observations from clinical experience and a number of infectious models indicate that the virulence of P. aeruginosa varies from strain to strain (Lee et al., 2006; Roy-Burman et al., 2001; Schulert et al., 2003; Woods et al., 1997), although the mechanisms accounting for this variation are not completely understood. The genes of most of the characterized P. aeruginosa virulence determinants are located in the core genome and therefore present in all strains (Wolfgang et al., 2003). Thus, it is conceivable that varying expression of these conserved pathogenic factors is responsible for differences in virulence between P. aeruginosa strains. Alternatively, P. aeruginosa's accessory genome may contribute to the heterogeneity of virulence. The accessory genome consists of bacteriophages, plasmids, and genomic islands found in some strains but not in others. Genomic islands in particular have been the focus of much recent attention. These horizontally transferred segments of DNA are often integrated into tRNA genes, have G+C contents divergent from that of the host core chromosome, and include components of mobile genetic elements (Cheetham et al., 1995; Dobrindt et al., 2004; Lawrence, 2005; Reiter et al., 1989). When they encode virulence determinants, genomic islands are referred to as pathogenicity islands (Dobrindt et al., 2004).

One well-described example of a pathogenicity island contributing to strain-to-strain variation in P. aeruginosa virulence is the family of islands that carry the exoU gene (Kulasekara et al., 2006), which encodes the type III secretion effector protein ExoU (Finck-Barbancon et al., 1997; Hauser et al., 1998). The exoU gene is present in approximately one-third of isolates obtained from acute infections, and secretion of the ExoU toxin is a marker for strains with enhanced virulence (Schulert et al., 2003). It is likely that additional pathogenicity islands contribute to the especially virulent phenotypes of some P. aeruginosa strains. If this is indeed the case, then highly virulent strains should prove to be rich sources of these islands. The identification of novel pathogenicity islands is important because they likely encode novel virulence determinants that would increase our understanding of P. aeruginosa pathogenesis.

Pseudomonas aeruginosa is a ubiquitous environmental gram-negative bacterium that can be found in lakes, streams, soil, and plant matter (Green et al., 1974; Hoadley, 1977; Rhame, 1979). In addition to thriving in multiple environmental niches, P. aeruginosa can infect many different organisms, including yeast (Hogan & Kolter, 2002), the nematode Caenorhabditis elegans (Mahajan-Miklos et al., 1999), insects (Jander et al., 2000), plants (Elrod & Braun, 1942; Rahme et al., 1995), and mammals (Glazebrook et al., 1978; Hammer et al., 2003). In humans, it is considered an opportunistic pathogen and is a significant cause of both acute infections (e.g. hospital-acquired pneumonia, urinary tract infections, and wound infections) and chronic infections (e.g. respiratory infections in individuals with cystic fibrosis) (Stryjewski & Sexton, 2003).

Two aspects of P. aeruginosa's genome evidently allow it to exploit differing environmental niches and infect a broad range of host organisms. First, it has an c. 6.3 Mb genome (Stover et al., 2000), one of the largest among bacteria. Thus, it harbors a large amount of genetic material necessary for environmental versatility. Consistent with its ability to inhabit diverse niches, P. aeruginosa's large genome has one of the highest proportions of predicted regulatory genes observed among bacterial genomes—8.4% of all predicted genes (Stover et al., 2000). Second, the P. aeruginosa genome contains a large number of genomic islands. About 90% of the P. aeruginosa chromosome is conserved (Wolfgang et al., 2003), but inserted within this core genome are genomic islands, which are found in some strains but not in others (Schmidt et al., 1996). Genomic islands are segments of DNA acquired by horizontal transfer (Dobrindt et al., 2004; Lawrence, 2005). They are frequently integrated adjacent to tRNA genes, have a G+C content distinct from that of the host core chromosome, and contain components of mobile genetic elements (Reiter et al., 1989; Cheetham & Katz, 1995). (Although the term ‘genomic island’ usually implies a large region of DNA, here it refers to both large and small segments of integrated DNA.) In P. aeruginosa, genomic islands constitute an accessory genome that may account for 10% of an individual isolate's genetic material (Spencer et al., 2003; Shen et al., 2006) and are thought to contribute to the ability of some P. aeruginosa strains to inhabit extreme environments.

Although the conserved core genome of P. aeruginosa has now been characterized by the sequencing of several strains (Stover et al., 2000; Lee et al., 2006; Mathee et al., 2008), the wealth of genetic material present in genomic islands remains relatively unexplored. Studies performed to date have identified and characterized several islands. For example, a 49 kb island called P. aeruginosa genomic island 1 (PAGI-1) was identified in a urinary tract infection isolate and was found to be present in 85% of the clinical strains tested (Liang et al., 2001). The large genomic islands PAGI-2 and PAGI-3 were identified by sequencing a hypervariable region in two different strains: a cystic fibrosis lung isolate and an environmental aquatic isolate (Larbig et al., 2002). Pseudomonas aeruginosa pathogenicity island-1 (PAPI-1) is representative of a large family of genomic islands derived from an ancestral pKLC102-like plasmid. pKLC102 is a 103.5-kb plasmid initially found in P. aeruginosa clone C strains that can exist as a plasmid or integrate into the chromosome, and can excise from the chromosome at a rate of up to 10% (He et al., 2004; Klockgether et al., 2004, 2007). A recent study comparing the genomes of five sequenced P. aeruginosa strains identified 62 genomic locations where at least one strain differed from the others by at least four ORFs (Mathee et al., 2008). These loci were designated ‘regions of genomic plasticity (RGPs)’ and represent hot spots for the presence of genomic islands. Therefore, characterized genomic islands represent a small fraction of the genomic diversity present in P. aeruginosa (Wolfgang et al., 2003).

Virulence is a complex trait requiring multiple steps, including entry into the host, adherence to and spread through host tissues, subversion of host defense systems, and induction of tissue damage (Finlay & Falkow, 1997). In P. aeruginosa, distinct strains appear to use a varying combination of factors to progress through these steps, and some of these factors appear to be encoded by genomic islands (Lee et al., 2006). This may suggest that unusually virulent strains of P. aeruginosa are likely to harbor a larger number of novel and interesting genomic islands. Thus, in a previous report, the virulence of a large collection of P. aeruginosa isolates was assessed in order to identify a candidate strain for studies aimed at identifying novel genomic islands (Battle et al., 2008). For this purpose, a set of 35 previously characterized P. aeruginosa clinical isolates (designated PSE isolates), all of which were originally cultured from patients with ventilator-associated pneumonia, was used (Hauser et al., 2002). Each isolate was screened for virulence in a mouse model of acute pneumonia and a lettuce leaf model of virulence in plants (Schulert et al., 2003; Battle et al., 2008). One isolate, PSE9, was noted to be virulent in both models and was chosen for further analysis. Subtractive hybridization was used to compare the genome of PSE9 with that of the less virulent but fully sequenced strain PAO1. This yielded 35 nonredundant sequences found in PSE9 but not in PAO1. Of these, 13 sequences corresponded to previously identified P. aeruginosa genetic elements. Seven novel islands were identified, one of which was designated P. aeruginosa genomic island 5 (PAGI-5) and examined further. This 99-kb island was shown to contain regions that were associated with highly virulent P. aeruginosa strains. Mutational analysis of these regions confirmed that they contributed to the highly virulent phenotype of the source strain. In addition to PAGI-5, an additional six PSE9 islands were identified by a similar approach and were designated PAGI-6, PAGI-7, PAGI-8, PAGI-9, PAGI-10, and PAGI-11.

Targeting of highly virulent bacterial strains may be a useful strategy for identifying novel genomic islands and virulence determinants. These determinants may be useful for identifying especially virulent strains of Pseudomonas spp. and may further be useful in diagnostic and therapeutic methods.

### SUMMARY

Disclosed are Pseudomonas aeruginosa Genomic Island (PAGI) nucleic acid sequences referred to as PAGI-5, PAGI-6, PAGI-7, PAGI-8, PAGI-9, PAGI-10, and PAGI-11 and the use thereof for detecting virulent strains of Pseudomonas bacteria. In some embodiments, these nucleic acid sequences may be useful in methods for identifying strains of Pseudomonas aeruginosa that comprise these nucleic acid sequences and exhibit increased virulence in comparison to strains that do not comprise these nucleic acid sequences.

The disclosed methods may be utilized to detect a virulent strain of Pseudomonas bacteria in a sample. The methods typically include detecting, either directly or indirectly, at least a fragment of PAGI-5, PAGI-6, PAGI-7, PAGI-8, PAGI-9, PAGI-10, or PAGI-11 nucleic acid in the sample, thereby detecting the virulent strain of Pseudomonas bacteria. In some embodiments, the methods include: (a) amplifying at least a fragment of PAGI-5, PAGI-6, PAGI-7, PAGI-8, PAGI-9, PAGI-10, or PAGI-11 from the sample to obtain amplified DNA; and (b) detecting the amplified DNA, thereby detecting the virulent strain of Pseudomonas bacteria. In other embodiments, the methods include (a) isolating nucleic acid from the sample; (b) contacting the isolated nucleic with an oligonucleotide that specifically hybridizes to nucleic acid of PAGI-5, PAGI-6, PAGI-7, PAGI-S, PAGI-9, PAGI-10, or PAGI-11; and (c) detecting hybridization of the oligonucleotide to the isolated nucleic acid, thereby detecting the virulent strain of Pseudomonas bacteria. In further embodiments, the methods include (a) isolating nucleic acid from the sample; (b) detecting a nucleic acid sequence in the isolated nucleic acid which comprises at least 10, 15, 20, 25, 30, 35, 40, 45, or 50 consecutive nucleotides of PAGI-5, PAGI-6, PAGI-7, PAGI-8, PAGI-9, PAGI-10, or PAGI-11, thereby detecting the virulent strain of Pseudomonas bacteria in the sample. Optionally, the methods include or do not include detecting PAGI-1, PAGI-2, PAGI-3, or PAGI-4 nucleic acid in the sample. Optionally, the methods include or do not include detecting at least a fragment of Pseudomonas aeruginosa pathenogenicity island 1 (PAPI-1) or (PAPI-2) (i.e., at least a fragment of PAPI-1 or PAPI-2), and in particular, at least a fragment of the exoU gene.

The methods may be utilized to identify a virulent strain of Pseudomonas bacteria (i.e., Pseudomonas spp.). In particular, the methods may be utilized to identify a virulent strain of Pseudomonas aeruginosa.

The methods may be utilized to identify a virulent strain of Pseudomonas bacteria in any suitable sample. Suitable samples may include biological samples from human patients.

The methods may include detecting at least a fragment of nucleic acid of PAGI-5, PAGI-6, PAGI-7, PAGI-8, PAGI-9, PAGI-10, or PAGI-11, or combinations thereof. Detecting may include amplifying DNA of PAGI-5, PAGI-6, PAGI-7, PAGI-8, PAGI-9, PAGI-10, or PAGI-11, or combinations thereof. In some embodiments, the amplified DNA may include at least about 50, 100, 150, 200, 250, 300, 400, 500, or 1000 contiguous nucleotides of PAGI-5, PAGI-6, PAGI-7, PAGI-8, PAGI-9, PAGI-10, or PAGI-1. For example, the amplified DNA may include at least about 50, 100, 150, 200, 250, 300, 400, 500, or 1000 contiguous nucleotides of PAGI-5 within novel region I (NR-I) or novel region II (NR-II). In some embodiments, the amplified DNA includes at least a portion of an ORF present in PAGI-5, PAGI-6, PAGI-7, PAGI-8, PAGI-9, PAGI-10, or PAGI-11, or combinations thereof. For example, the amplified DNA may include at least a portion of an ORF present in PAGI-5 within novel region I (NR-I) or novel region II (NR-II). Optionally, the methods include or do not include detecting a fragment of nucleic acid of PAGI-1, PAGI-2, PAGI-3, or PAGI-4 in the sample. Optionally, the methods include or do not include detecting at least a fragment of PAPI-1 or PAPI-2, and in particular include or do not include detecting at least a fragment of the exoU gene.

The methods may include contacting nucleic that is isolated from a sample with an oligonucleotide that specifically hybridizes to nucleic acid of PAGI-5, PAGI-6, PAGI-7, PAGI-8, PAGI-9, PAGI-10, or PAGI-11, or combinations thereof. The isolated nucleic acid may include DNA, which optionally may be amplified DNA. The oligonucleotide may include a label, for example the oligonucleotide may be conjugated to a fluorophore or a radioisotope. Hybridization of the oligonucleotide to the isolated nucleic acid may include detecting a signal from the label. In some embodiments, the isolated nucleic acid may be contacted with a pair of oligonucleotides that function as primers for amplifying at least a portion of the isolated nucleic acid to obtain amplified DNA. Detecting hybridization of the oligonucleotide may include detecting the amplified DNA. Optionally, the methods include or do not include contacting the isolated nucleic acid with an oligonucleotide that specifically hybridizes to nucleic acid of PAGI-1, PAGI-2, PAGI-3, or PAGI-4. Optionally, the methods include or do not include contacting the isolated nucleic acid with an oligonucleotide that specifically hybridizes to nucleic acid of PAPI-1 or PAPI-2, and in particular the exoU gene.

The disclosed methods may utilize one or more oligonucleotides that hybridize specifically to nucleic acid of PAGI-5, PAGI-6, PAGI-7, PAGI-8, PAGI-9, PAGI-10, or PAGI-11, or combinations thereof (e.g., as probes or primers for amplification). In some embodiments, the methods utilize one or more oligonucleotides that hybridize specifically to one or more ORFs present in PAGI-5, PAGI-6, PAGI-7, PAGI-8, PAGI-9, PAGI-10, or PAGI-11. In further embodiments, the methods utilize one or more oligonucleotides that hybridize specifically to nucleic acid of PAGI-5 within novel region I (NR-I) or novel region II (NR-II). The one or more oligonucleotides may hybridize specifically to one or more ORFs present within novel region I (NR-I) or novel region II (NR-II) of PAGI-5. Optionally, the oligonucleotides hybridize or do not hybridize specifically to nucleic acid of PAGI-1, PAGI-2, PAGI-3, or PAGI-4 (e.g., within an ORF contained therein). Optionally, the oligonucleotides hybridize or do not hybridize specifically to nucleic acid of PAPI-1 or PAPI-2, and in particular the exoU gene.

The disclosed methods may include detecting a nucleotide sequence in a nucleic acid isolated from a sample (which may include DNA that optionally has been amplified). The detected nucleotide sequence may include at least 10, 15, 20, 25, 30, 35, 40, 45, or 50 consecutive nucleotides of PAGI-5, PAGI-6, PAGI-7, PAGI-8, PAGI-9, PAGI-10, or PAGI-11, or combinations thereof. Detecting the nucleotide sequence may include contacting the isolated nucleic acid with an oligonucleotide that specifically hybridizes to nucleic acid of PAGI-5, PAGI-6, PAGI-7, PAGI-8, PAGI-9, PAGI-10, or PAGI-11. Optionally, the oligonucleotide may include a label and detecting hybridization of the oligonucleotide to the isolated nucleic acid may include detecting a signal from the label. Detecting the nucleotide sequence may include amplifying at least a portion of the isolated nucleic acid that includes the nucleotide sequence. In some embodiments, the detected nucleotide sequence may be present within novel region I (NR-I) or novel region II (NR-II) of PAGI-5. The detected nucleotide sequence may be present within an ORF (e.g., an ORF of PAGI-5, PAGI-6, PAGI-7, PAGI-8, PAGI-9, PAGI-10, or PAGI-11). Optionally, the detected nucleotide sequence includes or does not include 10, 15, 20, 25, 30, 35, 40, 45, or 50 consecutive nucleotides of PAGI-I, PAGI-2, PAGI-3, or PAGI-4. Optionally, the detected nucleotide sequence includes or does not include 10, 15, 20, 25, 30, 35, 40, 45, or 50 consecutive nucleotides of PAPI-1 or PAPI-2, and in particular the exoU gene.

The methods may include indirectly detecting at least a fragment of PAGI-5, PAGI-6, PAGI-7, PAGI-8, PAGI-9, PAGI-10, or PAGI-11 nucleic acid in the sample. For example, the methods may include detecting expression of at least a fragment of PAGI-5, PAGI-6, PAGI-7, PAGI-8, PAGI-9, PAGI-10, or PAGI-11 nucleic acid in the sample.

In some embodiments, the methods include: (a) reacting a sample with an antibody that binds specifically to a polypeptide encoded by an ORF present in PAGI-5, PAGI-6, PAGI-7, PAGI-8, PAGI-9, PAGI-10, or PAGI-11; and (b) detecting binding of the antibody to the polypeptide, thereby detecting the virulent strain of Pseudomonas bacteria in the sample. The antibody may include a label and detecting binding of the antibody to the polypeptide may include detecting a signal from the label. In some embodiments, the detected polypeptide may be encoded by an ORF present in PAGI-5 within novel region I (NR-I) or novel region II (NR-II). Optionally, the methods further may include or may not include reacting the sample with an antibody that binds specifically to a polypeptide encoded by an ORF present in PAGI-1, PAGI-2, PAGI-3, or PAGI-4. Optionally, the methods further may include or may not include reacting the sample with an antibody that binds specifically to a polypeptide encoded by an ORF present in PAPI-1 or PAPI-2, in particular the polypeptide encoded by the exoU gene.

Also disclosed are kits for performing the aforementioned methods. In some embodiments, the kits include one or more oligonucleotides for detecting or amplifying a nucleic acid sequence of PAGI-5, PAGI-6, PAGI-7, PAGI-8, PAGI-9, PAGI-10, or PAGI-11. The kits may include one or more oligonucleotides for detecting or amplifying a nucleic acid sequence of novel region I (NR-I) or novel region II (NR-II) of PAGI-5. The kits may include an antibody that binds specifically to a polypeptide encoded by an ORF present in PAGI-5, PAGI-6, PAGI-7, PAGI-8, PAGI-9, PAGI-10, or PAGI-11. In some embodiments, the kits include an antibody that binds specifically to a polypeptide encoded by an ORF present in PAGI-5 within novel region I (NR-I) or novel region II (NR-II).

### BRIEF DESCRIPTION OF THE DRAWINGS

FIG. 1. Virulence of P. aeruginosa isolates in a mouse model of acute pneumonia and distribution of PAGI-5 regions among these isolates. (A) Mice were infected with a range of bacterial inocula and monitored over the subsequent 7 days to calculate LD₅₀s. The y axis is inverted so that the results for more virulent isolates are indicated by taller bars. The gray bars indicate ExoU-secreting isolates, the diagonally striped bars indicate ExoS-secreting isolates, and the open bars indicate nonsecreting isolates. “Other” refers to isolate PSE7, which had a functional type III secretion system but did not secrete any effector proteins. (These results were previously published by Schulert et al. (2003) and are reproduced here in adapted form.) (B) Presence of PAGI-5 conserved and novel sequences in the panel of P. aeruginosa clinical isolates. The regions of PAGI-5 are indicated on the left, and a plus sign indicates the presence of the sequence.

FIG. 2. Virulence of P. aeruginosa isolates in a lettuce leaf model of infection. (A) Lettuce leaf infected with positive control strain PA14, clinical isolate PSE9, and reference strain PAO1. (B) Area of soft rot caused by each P. aeruginosa isolate normalized to the area of soft rot caused by PA14. The data are the means±standard errors of the means for three inoculation sites on a single leaf. For an explanation of the type III secretion profiles see the legend to FIG. 1.

FIG. 3. Flow chart showing the results of analysis of 75 PSE9 subtractive hybridization products. After removal of false positives, redundant clones, and sequences from previously characterized genomic islands, 22 distinct sequences remained. These sequences were used to screen a PSE9 genomic library.

FIG. 4. Map of PAGI-5. Arrows represent ORFs and are oriented in the direction of transcription. Gray arrows represent ORFs with similarity to PAPI-1 sequences, and open arrows represent ORFs that lack PAPI-1 similarity. Black arrows represent PAO1 ORFs that flank PAGI-5. Diagonal stripes indicate ORFs that are predicted to encode proteins with sequences that do not suggest a function, and speckled arrows indicate ORFs expected to play a role in DNA mobility. tRNA attL and attR sites are indicated by vertical arrows. G+C contents are indicated above the ORFs and were calculated using a sliding 100-bp window. PAGI-5 ORFs are designated “5PGX,” where “X” is the sequential number of the ORF within the genomic island.

FIG. 5. Alignment of PAGI-5, PAPI-1, and ExoU island A. Dark bands and ORFs represent conserved nucleotide sequences, whereas open ORFs indicate unrelated sequences. The double lines beneath PAGI-5 indicate the sequences that were amplified by PCR to detect the presence of the corresponding conserved and novel regions of PAGI-5 in the panel of 35 P. aeruginosa clinical isolates.

FIG. 6. Survival of PSE9, PSE9ΔNR-I, PSE9ΔNR-II, and PAO1 in a mouse model of acute pneumonia. The symbols indicate the percentage of animals surviving in each experimental group over time. Each group contained 14 to 35 mice pooled from at least two separate experiments. An asterisk indicates that values are significantly different (P=0.0036, log rank test).

FIG. 7. Results of competition assays using mixtures of PSE9 and PAO1, PSE9ΔNR-I, or PSE9ΔNR-II at 22 h post-infection for the lungs and spleen. Data from eight or nine mice from two experiments were pooled. Each symbol indicates the CI for the tissue sample from one mouse, and the bars indicate medians. CIs for parental strain PSE9 in competition with either PSE9ΔNR-I, PSE9ΔNR-II, or PAO1 were compared to CIs for parental strain PSE9 in competition with PSE9 tagged with a gentamicin resistance cassette to determine whether differences were significant. Statistical significance was determined using a two-tailed unpaired Student's I test (*, p≦0.05; **, P≦0.005).

FIG. 8. Strategy for identifying fosmid clones containing subtractive hybridization sequences. A three-tiered PCR-based screening process was used whereby pools of 96 fosmid clones were first screened for sequences found by subtractive hybridization. In the second screen, 12 pools of eight fosmid clones each were screened from each 96-well plate identified in the first screen. Finally, each individual clone from the pools identified in the second screen was itself screened for the presence of subtractive hybridization sequences.

FIG. 9. The PAGI-6 genomic island and map of PAGI-6. Arrows represent ORFs and are oriented in the direction of transcription. Arrows with gray backgrounds represent ORFs with similarity to φCTX sequences, and arrows with white backgrounds represent ORFs that lack φCTX similarity. Black arrows represent PAO1 ORFs that flank PAGI-6. ORFs without similarity to characterized ORFs are indicated with diagonal stripes, and ORFs expected to play a role in DNA mobility are speckled. tRNA attL and attR sites are represented by vertical arrows. The G+C content is shown above the ORFs, calculated from a sliding 100 bp window. PAGI-6 ORFs are referred to as ‘6PG#’, where ‘#’ is the sequential number of the ORF within the genomic island.

FIG. 10. PAGI-6 alignment with the φCTX genome. Dark bands and ORFs represent conserved nucleotide sequences, whereas light gray and white ORFs indicate unrelated sequences. att sites are represented by vertical arrows, and cos sites by circles.

FIG. 11. Map of PAGI-7. Arrows represent ORFs and are oriented in the direction of transcription. Arrows with white backgrounds represent PAGI-7 ORFs, and black arrows represent PAO1 ORFs that flank PAGI-7. ORFs without similarity to characterized ORFs are indicated by diagonal stripes, and ORFs expected to play a role in DNA mobility are speckled. The locations of inverted repeat sequences are indicated. The G+C content is shown above the ORFs, calculated from a sliding 100 bp window. PAGI-7 ORFs are referred to as ‘7PG#’, where ‘#’ is the sequential number of the ORF within the genomic island.

FIG. 12. Map of PAGI-8. Arrows represent ORFs and are oriented in the direction of transcription. Arrows with white backgrounds represent PAGI-8 ORFs, and black arrows represent PAO1 ORFs that flank PAGI-8. ORFs without similarity to characterized ORFs are indicated by diagonal stripes, and ORFs expected to play a role in DNA mobility are speckled. tRNA attL and attR sites are represented by vertical arrows. The G+C content is shown above the ORFs, calculated from a sliding 100 bp window. PAGI-8 ORFs are referred to as ‘8PG#’, where ‘#’ is the sequential number of the ORF within the genomic island.

FIG. 13. Maps of (a) PAGI-9, (b) PAGI-10, and (c) PAGI-11. Arrows with white backgrounds represent genomic island ORFs, and black arrows represent flanking PAO1 ORFs. The underlying gray bars indicate the extent of the PAO1 conserved sequence. Cross-hatching represents conserved PA14 sequence. The predicted locations of the Rhs element core extensions are indicated. The G+C content is shown above the ORFs, calculated from a sliding 100 bp window.

FIG. 14. Location of PSE9 genomic islands within the P. aeruginosa chromosome. The circular chromosome represents that of PAO1, which is 6,264,403 bp (Stover, et al., 2000). The actual chromosome of PSE9 has not been sequenced and may differ in size and include rearrangements relative to that of PAO1. The O-antigen cluster, the pilA gene, and PAGI-5, which were also identified by the subtractive hybridization approach, are described elsewhere (Battle, et al., 2008) but included here for completeness.

### DETAILED DESCRIPTION

The disclosed subject matter may be further described utilizing terms as defined below.

Unless otherwise specified or indicated by context, the terms “a”, “an”, and “the” mean “one or more.”

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean plus or minus ≦10% of the particular term and “substantially” and “significantly” will mean plus or minus >10% of the particular term.

As used herein, the terms “include” and “including” have the same meaning as the terms “comprise” and “comprising.”

The terms “patient” and “subject” may be used interchangeably herein. A patient may be a human patient. A patient may refer to a human patient having or at risk for acquiring an infection with Pseudomonas spp. (e.g., Pseudomonas aeruginosa). A “patient in need thereof” may include a patient having an infection with Pseudomonas spp. (e.g., Pseudomonas aeruginosa) or at risk for developing infection with Pseudomonas spp. (e.g., Pseudomonas aeruginosa).

The term “sample” is to be interpreted broadly to include patient samples and environmental samples. The term “patient sample” is meant to include biological samples such as tissues and bodily fluids. “Bodily fluids” may include, but are not limited to, blood, serum, plasma, saliva, cerebral spinal fluid, pleural fluid, tears, lactal duct fluid, lymph, sputum, and semen. Environmental samples may include, but are not limited to, surface swabs and water samples.

The term “nucleic acid” or “nucleic acid sequence” refers to an oligonucleotide, nucleotide or polynucleotide, which may include a full-length polynucleotide or a fragment or portion thereof. Nucleic acid may be single or double stranded, and represent the sense or antisense strand with respect to an encoded polypeptide. A nucleic acid may include DNA or RNA, and may be of natural or synthetic origin. For example, a nucleic acid may include mRNA or cDNA. Nucleic acid may include nucleic acid that has been reverse-transcribed and/or amplified (e.g., using polymerase chain reaction). A “fragment” of DNA typically comprises at least about 10, 15, 20, 25, 30, 35, 40, 45, or 50 nucleotides, and preferably at least about 50, 100, 150, 200, 250, 300, 400, 500, or 1000 nucleotides (which may be contiguous nucleotides relative to a reference sequence such as PAGI-5, PAGI-6, PAGI-7, PAGI-8, PAGI-9, PAGI-10, or PAGI-11, as further described herein, such as any of SEQ ID NOs:1-7.) The term “at least a fragment of” contemplates a full-length polynucleotide.

The term “source of nucleic acid” refers to any sample which contains nucleic acids (RNA or DNA). Particularly preferred sources of target nucleic acids are biological samples including, but not limited to blood, plasma, serum, saliva, cerebral spinal fluid, pleural fluid, milk, lymph, sputum and semen.

A “gene” refers to a DNA sequence that comprises control and coding sequences necessary for the production of an RNA, which may have a non-coding function (e.g., a ribosomal or transfer RNA) or which may include a polypeptide or a polypeptide precursor. As used herein the term “codon” refers to a sequence of three adjacent nucleotides (either RNA or DNA) constituting the genetic code that determines the insertion of a specific amino acid in a polypeptide chain during protein synthesis or the signal to stop protein synthesis. The term “codon” is also used to refer to the corresponding (and complementary) sequences of three nucleotides in the messenger RNA into which the original DNA is transcribed. An “open reading frame” or “ORF” refers to a consecutive series of codons that encodes a polypeptide. A gene for a polypeptide includes an ORF.

The term “oligonculeotide” is understood to be a molecule that has a sequence of bases on a backbone comprised mainly of identical monomer units at defined intervals. The bases are arranged on the backbone in such a way that they can enter into a bond with a nucleic acid having a sequence of bases that are complementary to the bases of the oligonucleotide. The most common oligonucleotides have a backbone of sugar phosphate units. A distinction may be made between oligodeoxyribonucleotides that do not have a hydroxyl group at the 2′ position and oligoribonucleotides that have a hydroxyl group in this position. Oligonucleotides of the method which function as primers or probes are generally at least about 8, 10, 12, or 14 nucleotide long and more preferably about 15 to 25 nucleotides long, although shorter or longer oligonucleotides may be used in the method. The exact size will depend on many factors, which in turn depend on the ultimate function or use of the oligonucleotide. The oligonucleotide may be generated in any manner, including chemical synthesis, DNA replication, reverse transcription, PCR, or a combination thereof. The oligonucleotide may be modified. For example, the oligonucleotide may be labeled with an agent that produces a detectable signal (e.g., a fluorophore or a radioisotope).

Oligonucleotides used as primers or probes for specifically amplifying (e.g., amplifying a particular target nucleic acid sequence) or specifically detecting (e.g., detecting a particular target nucleic acid sequence) generally are capable of specifically hybridizing to the target nucleic acid. An oligonucleotide (e.g., a probe or a primer) that is specific for a target nucleic acid will “hybridize” to the target nucleic acid under suitable conditions. As used herein, “hybridization” or “hybridizing” refers to the process by which an oligonucleotide single strand anneals with a complementary strand through base pairing under defined hybridization conditions.

As contemplated herein, an oligonucleotide that specifically hybridizes to PAGI-5, PAGI-6, PAGI-7, PAGI-8, PAGI-9, PAGI-10, or PAGI-10, may comprise a nucleic acid sequence (e.g., at least about 10, 15, 20, 25, 30, 35, 40, 45, or 50 nucleotides) that is the reverse complement of the corresponding sequence in PAGI-5, PAGI-6, PAGI-7, PAGI-8, PAGI-9, PAGI-10, or PAGI-11 to which the oligonucleotide specifically hybridizes. However, as contemplated herein, an oligonucleotide that specifically hybridizes to PAGI-5, PAGI-6, PAGI-7, PAGI-8, PAGI-9, PAGI-10, or PAGI-11 need not comprise the exact reverse complement of the corresponding sequence in PAGI-5, PAGI-6, PAGI-7, PAGI-8, PAGI-9, PAGI-10, or PAGI-11 to which the oligonucleotide specifically hybridizes. “Specific hybridization” is an indication that two nucleic acid sequences share a high degree of complementarity. Specific hybridization complexes form under permissive annealing conditions and remain hybridized after any subsequent washing steps. Permissive conditions for annealing of nucleic acid sequences are routinely determinable by one of ordinary skill in the art and may occur, for example, at 65° C. in the presence of about 6×SSC. Stringency of hybridization may be expressed, in part, with reference to the temperature under which the wash steps are carried out. Such temperatures are typically selected to be about 5° C. to about 20° C. lower than the thermal melting point (Tm) for the specific sequence at a defined ionic strength and pH. The Tm is the temperature (under defined ionic strength and pH) at which 50% of the target sequence hybridizes to a perfectly matched probe. Equations for calculating Tm and conditions for nucleic acid hybridization are known in the art.

“Primer” refers to an oligonucleotide that is capable of acting as a point of initiation of synthesis when placed under conditions in which primer extension is initiated (e.g., primer extension associated with an application such as PCR). An oligonucleotide “primer” may occur naturally, as in a purified restriction digest or may be produced synthetically. Primers contemplated herein may include, but are not limited to, oligonucleotides that comprise the nucleocleotide sequence of any of SEQ ID NOs:204-265.

A “probe” refers to an oligonucleotide that interacts with a target nucleic acid via hybridization. A probe may be fully complementary to a target nucleic acid sequence or partially complementary. The level of complementarity will depend on many factors based, in general, on the function of the probe. A probe or probes can be used, for example to detect the presence or absence of a mutation in a nucleic acid sequence by virtue of the sequence characteristics of the target. Probes can be labeled or unlabeled, or modified in any of a number of ways well known in the art. A probe may specifically hybridize to a target nucleic acid.

A “target nucleic acid” refers to a nucleic acid molecule containing a sequence that has at least partial complementarity with a probe oligonucleotide and/or a primer oligonucleotide. A primer or probe may specifically hybridize to a target nucleic acid. Target nucleic acid may refer to nucleic acid of PAGI-5, PAGI-6, PAGI-7, PAGI-8, PAGI-9, PAGI-10, PAGI-11, or combinations thereof (i.e., SEQ ID NOs:1-7, respectively).

The term “amplification” or “amplifying” refers to the production of additional copies of a nucleic acid sequence. Amplification is generally carried out using polymerase chain reaction (PCR) technologies known in the art. The term “amplification reaction system” refers to any in vitro means for multiplying the copies of a target sequence of nucleic acid. The term “amplification reaction mixture” refers to an aqueous solution comprising the various reagents used to amplify a target nucleic acid. These may include enzymes (e.g., a thermostable polymerase), aqueous buffers, salts, amplification primers, target nucleic acid, and nucleoside triphosphates, and optionally at least one labeled probe and/or optionally at least one agent for determining the melting temperature of an amplified target nucleic acid (e.g., a fluorescent intercalating agent that exhibits a change in fluorescence in the presence of double-stranded nucleic acid).

As used herein the term “sequencing” as in determining the sequence of a polynucleotide refers to methods that determine the base identity at multiple base positions or determine the base identity at a single position. “Detecting nucleic acid” as contemplated herein, may include “sequencing nucleic acid.”

The term “polypeptide” refers to a polymer of amino acids and fragments or portions thereof. A polypeptide may include amino acids of natural or synthetic origin. A “fragment” of a polypeptide, which alternatively may be called a peptide fragment, typically comprises at least about 10, 15, 20, 25, 30, 35, 40, 45, or 50 amino acids, and preferably at least about 50, 100, 150, 200, 250, 300, 400, 500, or 1000 amino acids (which may be contiguous amino acids relative to a reference amino acid sequence encoded by an ORF present in any of PAGI-5, PAGI-6, PAGI-7, PAGI-8, PAGI-9, PAGI-10, or PAGI-11, as further described herein, such as any of SEQ ID NOs:8-203 or the ORFs disclosed in Tables 2 and 6-9.) The term “at least a fragment of” contemplates a full-length polypeptide.

The term “Pseudomonas” or “Pseudomonas spp.” as used herein refers to any type of Pseudomonas bacteria, including but not limited to Pseudomonas aeruginosa, Pseudomonas putida, Pseudomonas fluorescens, and Pseudomonas multivorans. Particularly preferred for carrying out the present invention is Pseudomonas aeruginosa.

The terms “virulence” and “virulent” as used herein refers to the degree of pathogenicity of a microorganism, as indicated by fatality rate of infected hosts infected with that microorganism and/or the ability of that microorganism to invade the tissues of an infected host. For example, virulence may be assessed by determining the amount of bacteria which results in a 50% fatality rate in a given population of hosts (e.g., the LD50 in a population of mice). Relative virulence may refer to the virulence of a strain of Pseudomonas bacteria that comprises PAGI-5, PAGI-6, PAGI-7, PAGI-8, PAGI-9, PAGI-10, PAGI-11, or combinations thereof, in comparison to a strain of Pseudomonas bacteria that does not comprise Pseudomonas bacteria PAGI-5, PAGI-6, PAGI-7, PAGI-8, PAGI-9, PAGI-10, or PAGI-11. In some embodiments, a virulent strain of Pseudomonas bacteria may have an LD50 (e.g., in mice) that is no more than 1×10⁸ CFU, preferably no more than 1×10⁷ CFU, more preferably no more than 1×10⁶ CFU, even more preferably no more than 1×10⁵ CFU. For example, a highly virulent strain of Pseudomonas bacteria may have an LD50 in mice that is no more than about 1.3×10⁶ CFU as discussed below.

The term PAGI refers to “Pseudomonas aeruginosa Genomic Island.” As used herein, a “genomic island” refers to any chromosomal continuous fragment of DNA, regardless of size, that is found in some Pseudomonas aeruginosa strains but not others. The nucleic acid sequences for PAGI-5 (SEQ ID NO:1), PAGI-6 (SEQ ID NO:2), PAGI-7 (SEQ ID NO:3), PAGI-8 (SEQ ID NO:4), PAGI-9 (SEQ ID NO:5), PAGI-10 (SEQ ID NO:6), and PAGI-11 (SEQ ID NO:7) have been deposited at GenBank under accession nos. EF611301, EF611302, EF611303, EF6.1.1304, EF611305, EF611306, and EF611307, respectively, which GenBank entries are incorporated herein by reference in their entireties. The nucleic acid sequences for PAGI-1, PAGI-2, PAGI-3, and PAGI-4 have been deposited at GenBank under accession nos. AF241171, AF440523, AF440524, and AY258138, respectively, which GenBank entries are incorporated herein by reference in their entireties.

The methods contemplated herein may include detecting nucleic acid of an open reading frame (ORF) present within PAGI-5, PAGI-6, PAGI-7, PAGI-8, PAGI-9, PAGI-10, or PAGI-11. The methods contemplated herein also may include detecting a polypeptide encoded by an open reading frame present within PAGI-5, PAGI-6, PAGI-7, PAGI-8, PAGI-9, PAGI-10, or PAGI-11, which may include but are not limited to polypeptides comprising an amino acid sequence of any of SEQ ID NOs:8-203 or the ORFs disclosed in Tables 2 and 6-9. The methods may include detecting at least a fragment of a polypeptide encoded by an open reading frame present within PAGI-5, PAGI-6, PAGI-7, PAGI-8, PAGI-9, PAGI-10, or PAGI-11 (e.g., where the fragment comprises at least about a 10, 15, 20, 25, 30, 35, 40, 45, or 50 consecutive amino acid sequence of any of SEQ ID NOs:8-203 or the ORFs disclosed in Tables 2 and 6-9).

The methods contemplated herein may include detecting a polypeptide encoded by an open reading frame present within PAGI-5, PAGI-6, PAGI-7, PAGI-8, PAGI-9, PAGI-10, or PAGI-11, (e.g., a full-length polypeptide or a fragment thereof), by reacting the polypeptide or the fragment thereof with an antibody that specifically binds to the polypeptide or the fragment thereof. The term “antibody” is used in the broadest sense and specifically covers, for example, polyclonal antibodies, monoclonal antibodies, single chain antibodies, and antibody fragments. “Antibody fragments” comprise a portion of an intact antibody, preferably the antigen binding or variable region of the intact antibody. Examples of antibody fragments include Fab, Fab′, F(ab′)₂, and Fv fragments; diabodies; linear antibodies (Zapata et al., Protein Eng. 8(10): 1057-1062 [1995]); single-chain antibody molecules (i.e., scFv); and multispecific antibodies formed from antibody fragments. An antibody that “specifically binds to” or is “specific for” a particular polypeptide or an epitope on a particular polypeptide is one that binds to that particular polypeptide or epitope on a particular polypeptide without substantially binding to any other polypeptide or polypeptide epitope.

As used herein, a “label” refers to a detectable compound or composition which is conjugated directly or indirectly to an oligonucleotide or antibody so as to generate a “labeled” oligonucleotide or antibody. The label may be detectable by itself (e.g., radioisotope labels or fluorescent labels) or, in the case of an enzymatic label, may catalyze chemical alteration of a substrate compound or composition which then is detectable.

### ILLUSTRATIVE EMBODIMENTS

The following list of Embodiments is illustrative and is not intended to limit the scope of the claimed subject matter.

### Embodiment 1

A method for detecting a virulent strain of Pseudomonas bacteria in a sample, the method comprising detecting at least a fragment of PAGI-5, PAGI-6, PAGI-7, PAGI-8, PAGI-9, PAGI-10, or PAGI-11 nucleic acid in the sample, thereby detecting the virulent strain of Pseudomonas bacteria.

### Embodiment 2

The method of embodiment 1, comprising: (a) amplifying at least a fragment of PAGI-5, PAGI-6, PAGI-7, PAGI-8, PAGI-9, PAGI-10, or PAGI-11 from the sample to obtain amplified DNA; and (b) detecting the amplified DNA, thereby detecting the virulent strain of Pseudomonas bacteria.

### Embodiment 3

The method of embodiment 2, wherein the virulent strain of Pseudomonas bacteria is a virulent strain of Pseudomonas aeruginosa.

### Embodiment 4

The method of embodiment 2 or 3, wherein the sample is a biological sample from a patient.

### Embodiment 5

The method of any of embodiments 2-4, wherein the amplified DNA comprises at least about 100, 150, 200, 250, 300, 400, 500, or 1000 contiguous nucleotides of PAGI-5, PAGI-6, PAGI-7, PAGI-8, PAGI-9, PAGI-10, or PAGI-11.

### Embodiment 6

The method of any of embodiments 2-5, wherein the amplified DNA comprises at least about 100, 150, 200, 250, 300, 400, 500, or 1000 contiguous nucleotides of PAGI-5 within novel region I (NR-I) or novel region II (NR-II).

### Embodiment 7

The method of any of embodiments 2-5, wherein the amplified DNA comprises at least a portion of an ORF present in PAGI-5, PAGI-6, PAGI-7, PAGI-8, PAGI-9, PAGI-10, or PAGI-11.

### Embodiment 8

The method of embodiment 1, comprising: (a) isolating nucleic acid from the sample; (b) contacting the isolated nucleic with an oligonucleotide that specifically hybridizes to nucleic acid of PAGI-5, PAGI-6, PAGI-7, PAGI-8, PAGI-9, PAGI-10, or PAGI-11; and (c) detecting hybridization of the oligonucleotide to the isolated nucleic acid, thereby detecting the virulent strain of Pseudomonas bacteria.

### Embodiment 9

The method of embodiment 8, wherein the virulent strain of Pseudomonas bacteria is a virulent strain of Pseudomonas aeruginosa.

### Embodiment 10

The method of embodiment 8 or 9, wherein the sample is a biological sample from a patient.

### Embodiment 11

The method of any of embodiments 8-10, wherein the isolated nucleic acid comprises DNA.

### Embodiment 12

The method of any of embodiments 8-11, wherein the isolated nucleic acid comprises amplified DNA.

### Embodiment 13

The method of any of embodiments 8-12, wherein the oligonucleotide comprises a label and detecting hybridization of the oligonucleotide to the isolated nucleic acid comprises detecting a signal from the label.

### Embodiment 14

The method of any of embodiments 8-13, comprising contacting the isolated nucleic with a pair of oligonucleotides that function as primers and wherein detecting hybridization of the oligonucleotide to the isolated nucleic acid comprises amplifying at least a portion of the isolated nucleic acid.

### Embodiment 15

The method of any of embodiments 8-14, wherein the oligonucleotide hybridizes specifically to one or more ORFs present in PAGI-5, PAGI-6, PAGI-7, PAGI-8, PAGI-9, PAGI-10, or PAGI-11.

### Embodiment 16

The method of any of embodiments 8-15, wherein the oligonucleotide hybridizes specifically to nucleic acid of PAGI-5 within novel region I (NR-I) or novel region II (NR-II).

### Embodiment 17

The method of any of embodiments 8-16, wherein the oligonucleotide hybridizes specifically to one or more ORFs present within novel region I (NR-I) or novel region II (NR-II) of PAGI-5.

### Embodiment 18

The method of embodiment 1, comprising: (a) isolating nucleic acid from the sample; (b) detecting a nucleic acid sequence in the isolated nucleic acid which comprises at least 10, 15, 20, 25, 20, 35, 40, 45, or 50 consecutive nucleotides of PAGI-5, PAGI-6, PAGI-7, PAGI-8, PAGI-9, PAGI-10, or PAGI-11, thereby detecting the virulent strain of Pseudomonas bacteria in the sample.

### Embodiment 19

The method of embodiment 18, wherein the virulent strain of Pseudomonas bacteria is a virulent strain of Pseudomonas aeruginosa.

### Embodiment 20

The method of embodiment 18 or 19, wherein the sample is a biological sample from a patient.

### Embodiment 21

The method of any of embodiments 18-20, wherein the isolated nucleic acid comprises DNA.

### Embodiment 22

The method of any of embodiments 18-21, wherein the isolated nucleic acid comprises amplified DNA.

### Embodiment 23

The method of any of embodiments 18-22, wherein detecting comprises contacting the isolated nucleic acid with an oligonucleotide that specifically hybridizes to nucleic acid of PAGI-5, PAGI-6, PAGI-7, PAGI-8, PAGI-9, PAGI-10, or PAGI-11 and detecting hybridization of the oligonucleotide to the isolated nucleic acid.

### Embodiment 24

The method of embodiment 23, wherein the oligonucleotide comprises a label and detecting hybridization of the oligonucleotide to the isolated nucleic acid comprises detecting a signal from the label.

### Embodiment 25

The method of any of embodiments 18-24, wherein detecting comprises amplifying at least a portion of the isolated nucleic acid.

### Embodiment 26

The method of any of embodiments 18-25, wherein the detected nucleic acid sequence comprises at least 20 consecutive nucleotides of PAGI-5, PAGI-6, PAGI-7, PAGI-8, PAGI-9, PAGI-10, or PAGI-11.

### Embodiment 27

The method of any of embodiments 18-25, wherein the detected nucleic acid sequence comprises at least 30 consecutive nucleotides of PAGI-5, PAGI-6, PAGI-7, PAGI-8, PAGI-9, PAGI-10, or PAGI-11.

### Embodiment 28

The method of any of embodiments 18-25, wherein the detected nucleic acid sequence comprises at least 40 consecutive nucleotides of PAGI-5, PAGI-6, PAGI-7, PAGI-8, PAGI-9, PAGI-10, or PAGI-11.

### Embodiment 29

The method of any of embodiments 18-25, wherein the detected nucleic acid sequence comprises at least 50 consecutive nucleotides of PAGI-5, PAGI-6, PAGI-7, PAGI-8, PAGI-9, PAGI-10, or PAGI-11.

### Embodiment 30

The method of any of embodiments 18-25, wherein the detected nucleic acid sequence comprises at least 10 consecutive nucleotides of PAGI-5 within novel region I (NR-I) or novel region II (NR-II).

### Embodiment 31

The method of any of embodiments 18-25, wherein the detected nucleic acid sequence comprises at least 20 consecutive nucleotides of PAGI-5 within novel region I (NR-I) or novel region II (NR-II).

### Embodiment 32

The method of any of embodiments 18-25, wherein the detected nucleic acid sequence comprises at least 30 consecutive nucleotides of PAGI-5 within novel region I (NR-I) or novel region II (NR-II).

### Embodiment 33

The method of any of embodiments 18-25, wherein the detected nucleic acid sequence comprises at least 40 consecutive nucleotides of PAGI-5 within novel region I (NR-I) or novel region II (NR-II).

### Embodiment 34

The method of any of embodiments 18-25, wherein the detected nucleic acid sequence comprises at least 50 consecutive nucleotides of PAGI-5 within novel region I (NR-I) or novel region II (NR-II).

### Embodiment 35

The method of any of embodiments 18-25, wherein the detected nucleic acid sequence is present within an ORF of PAGI-5, PAGI-6, PAGI-7, PAGI-8, PAGI-9, PAGI-10, or PAGI-11.

### Embodiment 36

The method of any of embodiments 18-25, wherein the detected nucleic acid sequence is present within an ORF of PAGI-5 within novel region I (NR-I) or novel region II (NR-II).

### Embodiment 37

The method of any of embodiments 1-36, further comprising detecting, either directly or indirectly, at least a 10 nucleotide fragment of PAPI-1 or PAPI-2, and in particular at least a 10 nucleotide fragment of the exoU gene.

### Embodiment 38

A method for detecting a virulent strain of Pseudomonas bacteria in a sample, the method comprising: (a) reacting the sample with an antibody that binds specifically to a polypeptide encoded by an ORF present in PAGI-5, PAGI-6, PAGI-7, PAGI-8, PAGI-9, PAGI-10, or PAGI-11; and (b) detecting binding of the antibody to the polypeptide, thereby detecting the virulent strain of Pseudomonas bacteria in the sample.

### Embodiment 39

The method of embodiment 38, wherein the virulent strain of Pseudomonas bacteria is a virulent strain of Pseudomonas aeruginosa.

### Embodiment 40

The method of embodiment 38 or 39, wherein the sample is a biological sample from a patient.

### Embodiment 41

The method of any of embodiments 38-40, wherein the antibody comprises a label and detecting binding of the antibody to the polypeptide comprises detecting a signal from the label.

### Embodiment 42

The method of any of embodiments 38-42, wherein the detected polypeptide is encoded by an ORF present in PAGI-5 within novel region I (NR-I) or novel region II (NR-II).

### Embodiment 43

The method of any of embodiments 38-42, further comprising reacting the sample with an antibody that binds specifically to a polypeptide encoded by an ORF present in PAPI-1 or PAPI-2, in particular the polypeptide encoded by the exoU gene.

### Embodiment 44

The method of any of embodiments 1-43, wherein the virulent strain of Pseudomonas bacteria has an LD50 in mice that is no more than 1×10⁵ CFU, 1×10⁶ CFU, 1×10⁷ CFU, or 1×10⁸ CFU.

### Embodiment 45

A kit for performing any of the methods of embodiments 1-44, comprising an oligonucleotide for detecting a nucleic acid sequence of PAGI-5, PAGI-6, PAGI-7, PAGI-8, PAGI-9, PAGI-10, or PAGI-11.

### Embodiment 46

The kit of embodiment 45, comprising an oligonucleotide for detecting a nucleic acid sequence of novel region I (NR-I) or novel region II (NR-II) of PAGI-5.

### Embodiment 47

A kit for performing any of the methods of embodiments 38-44, comprising an antibody that binds specifically to a polypeptide encoded by an ORF present in PAGI-5, PAGI-6, PAGI-7, PAGI-8, PAGI-9, PAGI-10, or PAGI-11 .

### Embodiment 48

A kit of embodiment 47, comprising an antibody that binds specifically to a polypeptide encoded by an ORF present in PAGI-5 within novel region I (NR-I) or novel region II (NR-II).

### EXAMPLES

The following Examples are illustrative and are not intended to limit the scope of the claimed subject matter.

### Example 1

Reference is made to the scientific article Battle et al., “Hybrid Pathogenicity Island PAGI-5 Contributes to the Highly Virulent Phenotype of a Pseudomonas aeruginosa Isolate in Mammals,” J. Bacteriol. 2008 November; 190(21):7130-40. Epub 2008 Aug. 29, the content of which is incorporated herein by reference in its entirety.

### SUMMARY

Most known virulence determinants of Pseudomonas aeruginosa are remarkably conserved in this bacterium's core genome, yet individual strains differ significantly in virulence. One explanation for this discrepancy is that pathogenicity islands, regions of DNA found in some strains but not in others, contribute to the overall virulence of P. aeruginosa. Here, the virulence of a panel of P. aeruginosa isolates was tested in mouse and plant models of disease, and a highly virulent isolate, PSE9, was chosen for comparison by subtractive hybridization to a less virulent strain, PAO1. The resulting subtractive hybridization sequences were used as tags to identify genomic islands found in PSE9 but absent in PAO1. One 99-kb island, designated P. aeruginosa genomic island 5 (PAGI-5), was a hybrid of the known P. aeruginosa island PAPI-1 and novel sequences. Whereas the PAPI-1-like sequences were found in most tested isolates, the novel sequences were found only in the most virulent isolates. Deletional analysis confirmed that some of these novel sequences contributed to the highly virulent phenotype of PSE9. These results indicate that targeting highly virulent strains of P. aeruginosa may be a useful strategy for identifying pathogenicity islands and novel virulence determinants.

Materials and Methods

Bacterial strains and growth conditions. P. aeruginosa PSE strains PSE1 to PSE35 were previously obtained by culture of bronchoscopic fluid from patients who met strict criteria for ventilator-associated pneumonia (Hauser et al., 2002). PAO1 is a laboratory strain of P. aeruginosa (Holloway et al., 1979), and PA14 is a human clinical isolate known to be pathogenic in both plants and mammals (Rahme et al., 2000). Escherichia coli strains JM109 (Promega, Madison, Wis.), EP1300-T1R (Epicentre, Madison, Wis.), and S17.1 (Simon et al., 1983) were used for cloning and conjugation experiments. Antibiotic concentrations and growth conditions are described below.

Mouse model of acute pneumonia. Data from experiments in which mice were infected with PSE strains were published previously (Schulert et al., 2003) and are reproduced here with permission to facilitate comparison with data from plant virulence studies. The mice were infected intranasally as previously described (Schulert et al., 2003).

Mouse survival studies were performed as previously described by Comolli et al. (Comolli et al., 1999). Briefly, bacteria grown for 17 h in MINS medium (Nicas et al., 1984) at 37° C. with shaking (250 rpm) were diluted, regrown to exponential phase, and then were washed and resuspended in phosphate-buffered saline (PBS) (Invitrogen). Six- to eight-week-old female BALB/c mice were anesthetized by intraperitoneal injection of a mixture of ketamine (100 mg/ml) and xylazine (20 mg/ml). A bacterial dose that was approximately equal to the 50% lethal dose (LD50) of PSE9 in 50 ml PBS, as determined by measuring the optical density and confirmed by plating serial dilutions onto Vogel-Bonner medium (VBM) agar, was instilled into the noses of anesthetized mice. The mice were monitored for survival or severe illness over the next 7 days. Severely ill mice, as determined by the presence of matted fur, labored breathing, and decreased activity, were euthanized and scored as dead. The experiments were performed twice, and the results were pooled.

For competition experiments, mice were inoculated as described above for the survival experiments. Inoculation was performed using approximately equal numbers (as determined by measuring the optical density and by plating to obtain viable counts) of parental strain PSE9 and a deletion mutant strain or approximately equal numbers of wild-type strain PAO1 and a PSE9 strain tagged with a gentamicin resistance cassette to allow discrimination between PSE9 and PAO1. Mice were re-anesthetized and sacrificed at 22 h post-infection. Lungs and spleens were aseptically removed prior to homogenization in 5 ml PBS. The bacterial load in each organ was determined following plating of serial dilutions on Luria-Bertani (LB) agar and LB agar supplemented with 100 μg/ml of gentamicin to distinguish PSE9 from the second bacterial strain. Colonies were counted following incubation at 37° C. for 24 h. The following formula was used to calculate the competitive index (CI) (Logsdon et al., 2003): CI=(mutant/wild-type output ratio)/(mutant/wild-type input ratio).

All experiments were approved by and performed in accordance with the guidelines of the Northwestern University Animal Care and Use Committee.

Lettuce infection model. The lettuce infection model was adapted from the model described by Rahme and colleagues (Rahme et al., 1997). Briefly, P. aeruginosa strains were grown to saturation in LB broth at 37° C. Cultures were then diluted 1:200 in fresh LB broth and grown for an additional 3 to 4 h. The resulting log-phase cultures were diluted in 10 mM MgSO4 to obtain an optical density at 600 nm of 0.2. Romaine lettuce leaves were purchased from a local supermarket, washed in 0.1% bleach, rinsed with water, and then placed in a plastic container lined with Whatman paper impregnated with MgSO4. A pipette tip was used to puncture the lettuce midrib and inoculate 10 μl of a diluted culture. The leaves were incubated at 30° C. in a humid environment for 4 days, after which the length and width of the region of soft rot were measured. The area of soft rot was estimated using the following formula: A=0.25π×l×w, where A is area of tissue damage, l is the length, and w is the width. Each strain was inoculated in triplicate. The area of soft rot caused by each P. aeruginosa isolate inoculated was compared to the area of soft rot caused by PA14 inoculated adjacently to control for leaf-to-leaf variation. In certain experiments, the number of CFU present within a lettuce lesion was determined by a method adapted from the method of Dong et al. (Dong et al., 1991). Briefly, after 4 days the infected region of a lettuce leaf was cut from the midrib and macerated in 5 ml of 10 mM MgSO4 with a mortar and pestle. Serial dilutions were plated on LB agar for enumeration of bacterial CFU following incubation at 37° C. for 24 h.

Subtractive hybridization. Bacterial genomic DNA was purified from P. aeruginosa strains PSE9 and PAO1 using Genomic-Tip 500/G columns (Qiagen, Valencia, Calif.) by following the manufacturer's instructions. Subtractive hybridization was then performed using the PCR-Select bacterial genome subtractive hybridization approach (Clontech, Mountain View, Calif.). Subtractive hybridization was performed as directed by the manufacturer except for the following changes. Genomic DNA was ethanol precipitated with a linear acrylamide carrier (Bio-Rad, Hercules, Calif.) (Gaillard et al. (1990)). The primary PCR mixture was incubated at 72° C. for 5 min to allow filling of the adapter overhangs before incubation at 94° C. for 30 s, at 56° C. for 30 s, and at 72° C. for 90 s for 25 cycles. The secondary PCR mixture was heated to 72° C. before addition of Taq polymerase. The sample was then incubated at 94° C. for 30 s, at 58° C. for 30 s, and at 72° C. for 90 s for 15 cycles. PCR products were purified using the QIAquick PCR purification approach (Qiagen).

Generation of the subtractive hybridization library. Subtractive hybridization products were ligated to the pGEM-T T/A cloning vector (Promega) at 4° C. overnight (Sambrook et al., 1989). Transformation was performed by adding 2 μl of a ligation mixture to JM109 competent cells (Sambrook et al., 1989), and transformants were selected for by growth on LB agar supplemented with ampicillin (50 μg/μl), isopropyl-β-d-thiogalactopyranoside (IPTG) (50 μg/μl), and 5-bromo-4-chloro-3-indolyl-β-d-galactopyranoside (X-Gal) (50 μg/μl; Sigma-Aldrich, St. Louis, Mo.). Following growth in LB broth supplemented with ampicillin (50 μg/μl), plasmid DNA was purified from selected transformants using a spin column technique (Qiagen). Plasmid DNA was digested with BglI for 1 h at 37° C. and then screened to determine the presence of an insert and the insert size following electrophoresis through a 0.8% agarose gel. Plasmids containing inserts were sequenced by the University of Chicago Cancer Research Center DNA Sequencing Facility (Chicago, Ill.).

PSE9 genomic library. To generate a PSE9 genomic library, the fosmid vector pSB100 was first constructed as follows. The 4.35-kb DrdI fragment of plasmid mini-CTX1 (Hoang et al., 2000), which encodes tetracycline resistance, has an oriT site for mating into P. aeruginosa, and has an attP site and integrase gene for integration into an intergenic chromosomal attB site on the P. aeruginosa chromosome, was purified. This fragment was treated with the DNA polymerase Klenow fragment (New England Biolabs, Beverly, Mass.) along with each deoxynucleoside triphosphate at a concentration of 33 μM to generate blunt ends and was ligated into the blunt Eco72 I site of the fosmid pCC1FOS (Epicentre) to generate pSB100. The pCC1 FOS vector contributed chloramphenicol resistance and cos, ori2, and oriV sites to pSB100. ori2 is the E. coli F-factor single-copy origin of replication, and oriV is an inducible high-copy-number origin of replication. These ori sites allowed pSB100 to be maintained as a low-copy-number fosmid yet to be induced to high copy numbers to facilitate fosmid DNA purification.

To construct a fosmid library of PSE9 genomic DNA fragments, the vector pSB100 was digested with XhoI, and overhangs were partially filled with dTTP and dCTP to generate ends with 5′ TC overhangs. PSE9 genomic DNA was purified as described above, and 1 μg of DNA was partially digested with 0.3 U of Sau3AI (New England Biolabs) at 37° C. for 1 h, which was followed by heat inactivation for 20 min at 60° C. To generate DNA fragments with GA 5′ overhangs compatible with the TC 5′ overhangs of the modified pSB100 fosmid vectors, 2.5 U of the DNA polymerase Klenow fragment was added, and the reaction mixture was incubated at 25° C. for 15 min in the presence of 33 mM dATP and dGTP. The reaction was terminated by addition of 1.5 μl of 0.2 M EDTA and by heat inactivation at 75° C. for 15 min. Following electrophoresis of eight of the reaction mixtures described above through a 0.6% low-melting-point agarose gel, DNA fragments that ranged from 25 to 40 kb long were extracted from the gel using the GELase enzyme preparation (Invitrogen, Carlsbad, Calif.). Extracted DNA was precipitated with ethanol.

The digested vector and insert were ligated by incubation with Fast-Link DNA ligase (Epicentre) at room temperature for 2 h. The ligation reaction mixture was then incubated with MaxPlax lambda packaging extract (Epicentre) and transduced into E. coli strain EPI300-T1R (Epicentre). Bacteria were plated on LB agar supplemented with chloramphenicol (12.5 μg/μl). A total of 960 colonies were individually inoculated into the wells of 96-well plates containing 150 μl/well LB broth supplemented with glycerol (7.5%) and chloramphenicol (12.5 μg/μl). Ten 96-well plates were incubated at 37° C. overnight in a nonshaking incubator and then stored at −80° C.

To assess the quality of the fosmid library, fosmid DNA was isolated from 25 randomly selected clones. The DNA was digested with HindIII, and the restriction digestion patterns were examined following electrophoresis through an agarose gel (0.8%). From the restriction pattern of these fosmid clones, it was estimated that the fosmid insert sizes were between 30 kb and 40 kb. Conservatively assuming an average insert size of 30 kb, a library of this size would be predicted to have a 99% probability of containing any particular genomic sequence.

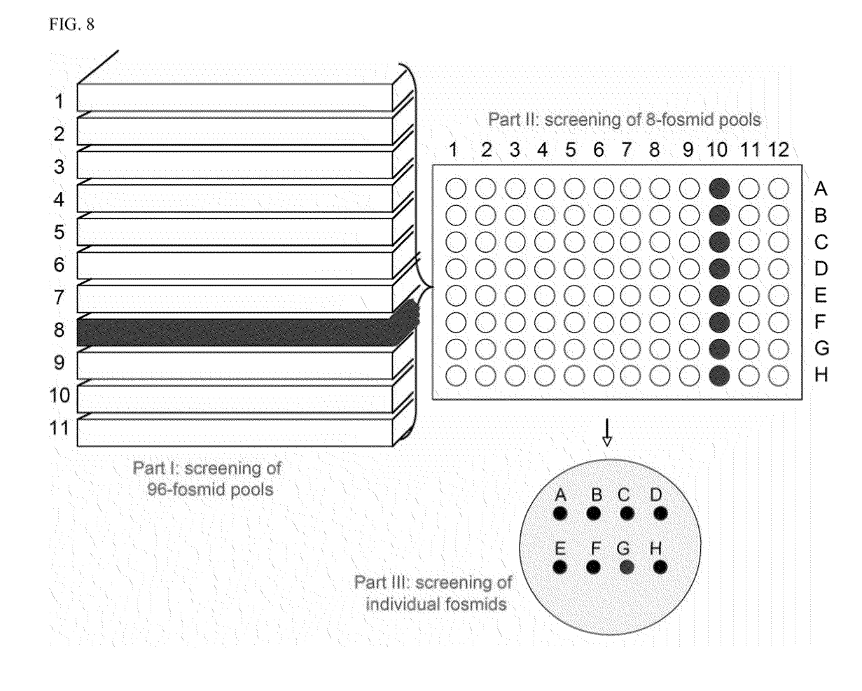

Screening the fosmid library for subtractive hybridization sequences. Fosmid library clones containing subtractive hybridization sequences were detected using a three-tiered PCR-based screening approach (see FIG. 8). In the first screening step, PCR amplification using primers specific for subtractive hybridization products (see Table 3) was performed with pools of fosmid clones, each pool consisted of all the fosmid clones from a 96-well plate. Fosmid pools were created by replica plating the stocks onto LB agar supplemented with chloramphenicol (12.5 μg/ml), followed by incubation overnight at 37° C. Pools of bacteria were collected directly from the plates by washing with STET buffer (0.1 M NaCl, 10 mM Tris, 1 mM EDTA, 5% Triton X-100) and centrifugation at 18,000×g. Pools of DNA were then isolated using a column spin technique (Qiagen). Thus, the entire 960-member fosmid library was represented by 10 pools of DNA, which were used as templates for the PCRs. The reaction mixtures were incubated at 94° C. for 30 s, at 52° C. for 30 s, and at 72° C. for 45 s for 25 cycles. In the second screening step, 12 fosmid pools were created using the eight clones from each column of wells in each 96-well plate that had tested positive in the first step of the screen. PCR amplification was then performed to identify which column pool contained the clone of interest. Thus, the location of the library clone containing a specific subtractive hybridization sequence was narrowed to a column of a 96-well plate by screening 12 pools. In the final step of the screening process, each of the eight individual clones from the identified column pool was individually screened by PCR amplification. In this way, the entire fosmid library was rapidly screened for the presence of subtractive hybridization sequences.

Sequencing of fosmids. To obtain the sequences of the inserts in fosmids containing subtractive hybridization sequences, the EZ::TN<KAN-2> transposon-mediated sequencing approach was used (Epicentre). Briefly, 0.05 pmol of transposon EZ::TN<KAN-2> (provided by the manufacturer) and 2 μg of fosmid DNA were incubated with EZ::TN transposase (provided by the manufacturer) at 37° C. for 2 h. The reaction was terminated with stop solution (provided by the manufacturer), and 1 μl of the reaction mixture was electroporated into electrocompetent E. coli EP1300-T1R cells (Epicentre). Electroporated E. coli cells were plated onto LB agar supplemented with kanamycin (50 μg/ml). Colonies were inoculated into 1 ml LB broth supplemented with kanamycin (50 μg/ml) and grown overnight. Cultures were added to 9 ml of LB medium supplemented with chloramphenicol (12.5 μg/ml) and 10 μl CopyControl induction solution (provided by manufacturer), which induces fosmids to high copy numbers, and shaken at 37° C. for 5 h. Fosmid DNA was purified using a spin column approach (Qiagen). Primers hybridizing to the borders of the transposon were used to sequence the DNA flanking the transposon insertion site (primer KAN-2 FP-1, ACCTACAACAAAGCTCTCATCAACC (SEQ ID NO:256); primer KAN-2 RP-1, GCAATGTAACATCAGAGATTTTGAG (SEQ ID NO:257)), and primer walking was used to fill in sequence gaps. Sequencing was performed by SeqWright (Dallas, Tex.) and by the University of Chicago Cancer Research Center DNA Sequencing Facility. Sequences not present in the fosmid library were obtained by PCR amplification of chromosomal DNA using the Advantage-GC genomic polymerase mixture (Clontech). Each strand of DNA was sequenced one or two times.

Sequence assembly, annotation, and analysis. Contiguous sequences were assembled using Vector NTI Contig Express (InforMax, Inc., Frederick, Md.). Open reading frames (ORFs) in genomic island sequences were predicted using GenDB (Meyer et al., 2003) and GeneMark (Lukashin et al., 1998), and the G+C content was calculated by using Vector NTI BioPlot (InforMax, Inc.) from a sliding 100-bp window. Nucleotide and amino acid sequence similarities were identified using BLASTN and BLASTP, respectively (Altschul et al., 1990), and sequences were aligned using Vector NTI AlignX (Informax, Inc.).

Construction of the PAGI-5 deletion mutant. PSE9 mutants with deletion of novel region I (NR-I) of PAGI-5 (PSE9ΔNR-I) or novel region II (NR-II) of PAGI-5 (PSE9ΔNR-II) were created by homologous recombination using a variation of the method of Schweizer and Hoang (Schweizer et al., 1995). PCR primers were designed to amplify 500 to 700-bp fragments of the 5′ and 3′ ends of PAGI-5 NR-I and NR-II. These PCR fragments were engineered to have NgoMIV restriction sites on the exterior side and XmaI sites on the interior side. These PCR products were digested with NgoMIV and XmaI and sequentially cloned into the XmaI site of pEX100T (Schweizer et al., 1995). After PCR was used to confirm the correct orientation of the cloned fragments, the 2.3-kb XmaI digestion product of pX1918G (Schweizer et al., 1995), which contained a gentamicin resistance cassette, was cloned into the Xmal site between the two fragments, creating deletion vectors pPG5NRI-5G3 and pPG5NRII-5G3. The deletion vectors were transformed into E. coli S17.1 and then mated into PSE9 (Schweizer et al., 1995). Selection for vector integration into the PSE9 genome was obtained by growth on VBM (Vogel et al., 1956) agar supplemented with 100 μg/ml gentamicin. Gentamicin-resistant colonies were transferred to VBM agar supplemented with 100 μg/ml gentamicin and 5% sucrose to induce a second recombination event that resulted in deletion of the targeted region as well as the vector backbone, which included the sacB sucrose sensitivity gene. PCR was used to screen gentamicin- and sucrose-resistant colonies for the presence of the gentamicin resistance cassette (X1918G-GentF, CGCAGCAGCAACGATGTTACGC (SEQ ID NO:258); X1918G-GentR, CGCGTTGGCCTCATGCTTGA (SEQ ID NO:259); X1918G-XylF, TCGAATTCCTCCGCGAGAGC (SEQ ID NO:260); X1918G-XylR, AAATCCATGCCCGGCTCGTC (SEQ ID NO:261)) and deletion of NR-I (PAGI5-5Gupstream, GCACGTTGCCAGATGTTCTCC (SEQ ID NO:262); PAGI5-5Gdownstream, GGCAGAAATGGCTGCGTTCG (SEQ ID NO:263)) and NR-II (PAGI5-MGupstream, CGATTCAAGCGAGCCAGGATC (SEQ ID NO:264); PAGI5MGdownstream, GCCACCACGTTGACAACAAGCT (SEQ ID NO:265)).

Construction of a gentamicin-resistant PSE9 strain. To distinguish PSE9 from PAO1 in competition experiments, it was necessary to tag PSE9 with a chromosomal copy of a gentamicin resistance cassette. The 2.3-kb XmaI fragment from pX1918G (Schweizer et al., 1995) containing the gentamicin resistance cassette was cloned into the Xmal site of mini-CTX1 (Hoang et al., 2000). The resulting mini-CTX-Gent construct was transformed into E. coli S17.1 and mated into wild-type strain PSE9, in which it integrated into the chromosomal attB site. The vector backbone was then excised by mating pFlp2 (Hoang et al., 2000) into the strain, resulting in expression of Flp recombinase. Integration and vector excision were confirmed by PCR as described above for the PSE9ΔNR-I and PSE9ΔNR-II mutants. The absence of a virulence defect in the tagged PSE9 strain was confirmed by performing competition experiments with tagged PSE9 and parental strain PSE9 (data not shown).

Sequencing of PAGI-5. The PAGI-5 genomic island was sequenced as follows: Two of the subtractive hybridization sequences that lacked similarity to known sequences identified two overlapping fosmids. Complete sequencing of the inserts in these fosmids indicated that the novel subtractive hybridization sequences were contiguous with sequences related to PAPI-1. Rescreening the fosmid library with PCR primers designed to amplify other PAPI-1 sequences identified a third non-overlapping fosmid containing a PAPI-1 related region contiguous with PAO1 backbone sequence. Long-range PCR using PSE9 chromosomal DNA as template was utilized to amplify the sequence between the PAPI-1 related regions of the two overlapping fosmids and those of the third fosmid. Sequencing of the amplified product indicated that it contained the PAPI-1 subtractive hybridization sequence, which had not been found in the fosmid library. In this way, the complete sequence of PAGI-5 was obtained and localized within the core chromosome.

Nucleotide sequence accession number. The sequence of PAGI-5 has been deposited in the National Center for Biotechnology Information GenBank database under accession number EF611301.

Results and Discussion

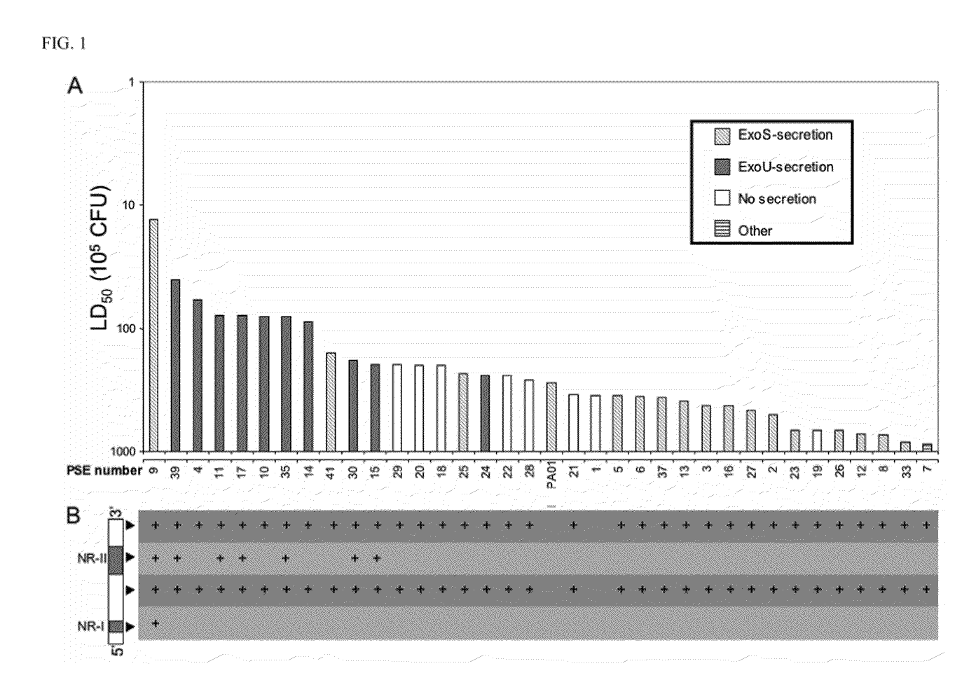

Virulence in a mouse model of pneumonia. We first investigated strain-to-strain variation in the virulence of P. aeruginosa. For this purpose, a set of 35 previously collected P. aeruginosa clinical isolates designated PSE1, PSE2, PSE3, etc., was used (Hauser et al., 2002). Each of these isolates was originally cultured from patients with ventilator-associated pneumonia. The virulence of these 35 isolates was previously quantified in a mouse model of acute pneumonia by calculating the LD50 (Schulert et al., 2003). As an aid, the data are shown in FIG. 1A.

Significant strain-to-strain variation in the levels of virulence was observed, and the LD50s of the most and least virulent strains differed by almost 100-fold. The most virulent strain was PSE9, which had an LD50 of 1.3×10⁶ CFU, while the least virulent strain was PSE7, which had an LD50 of 8.8×10⁷ CFU. The laboratory strain PAO1 was used as a control and was found to have an intermediate level of virulence (LD50, 4.2×10⁷ CFU). These results confirm that strains of P. aeruginosa differ in virulence in an animal model of infection.

The difference in pathogenicity of P. aeruginosa strains suggested that some strains might possess virulence factors that other strains lack. Although the genes encoding most known P. aeruginosa virulence factors are conserved in nearly all strains (Wolfgang et al., 2003), the exoU and exoS genes, which encode effector proteins of the P. aeruginosa type III secretion system, are variable traits (Feltman et al., 2001; Fleiszig et al., 1997). For this reason, the type III secretion profile of each of the 35 strains was determined previously (Schulert et al., 2003); this analysis showed that ExoU-secreting strains as a group were indeed more virulent (FIG. 1A), but neither ExoU nor ExoS secretion explained all the differences in virulence between these strains (Schulert et al., 2003). Therefore, it was postulated that the remaining differences were due to either differential regulation of conserved virulence determinants or the variable presence of virulence-encoding genes in the accessory genomes of the strains. Subsequent experiments focused on the latter set of genes.

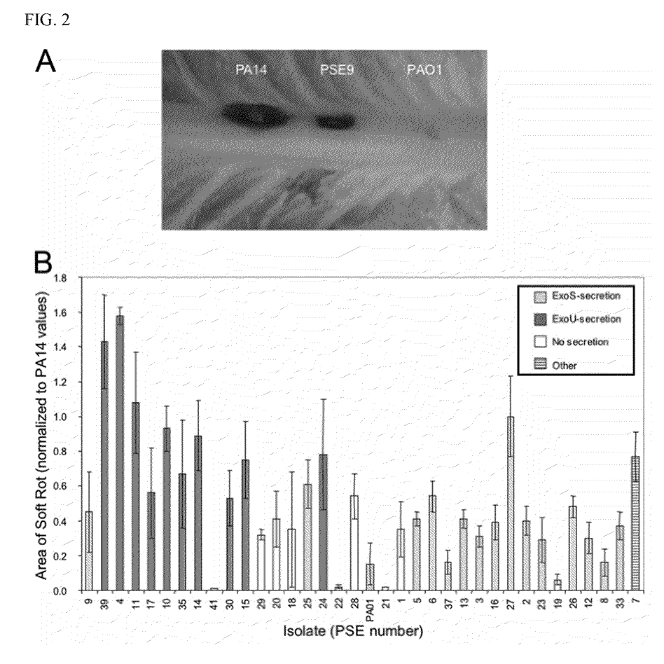

Virulence in a plant model of infection. Since P. aeruginosa is also a pathogen of plants (He et al., 2004; Rahme et al., 1995), the virulence of the 35 isolates was quantified using a plant model of disease. The lettuce leaf infection system developed by Rahme and colleagues was used for this purpose (Rahme et al., 1997). P. aeruginosa was inoculated into the spines of lettuce leaves, and the areas of tissue damage that developed over the ensuing 4 days were determined and used to quantify virulence (FIG. 2A). Strain P A14, a clinical isolate known to be highly virulent in plants (Rahme et al., 1995), was used as a positive control. Again, the 35 isolates differed in virulence (FIG. 2B). Whereas some strains had no apparent effect on the lettuce, other strains caused areas of tissue damage larger than those caused by strain PA14. PAO1 was relatively avirulent in this model system, producing an area of damage that was only 15% of the area of damage produced by PA14. Some of the strains that were highly virulent in the mouse model exhibited low levels of virulence in the lettuce model (e.g., PSE41), while other strains were highly virulent in the lettuce model but only slightly virulent in the mouse model (e.g., PSE7 and PSE27). Still other strains were highly virulent in both models (e.g., PSE39 and PSE4). The most virulent isolate in the mouse model, PSE9, was the 17th most virulent strain in the plant model and caused an area of damage that was just under one-half the area of damage caused by PA14. These findings confirm those of Rahme et al. (Rahme et al., 2000) and demonstrate that strains of P. aeruginosa differ in virulence in a plant model of infection. Furthermore, they are consistent with a model in which the accessory genetic material of some strains enhances virulence in animals, the accessory genetic material of other strains enhances virulence in plants, and the accessory genetic material of still other strains enhances virulence in both animals and plants.

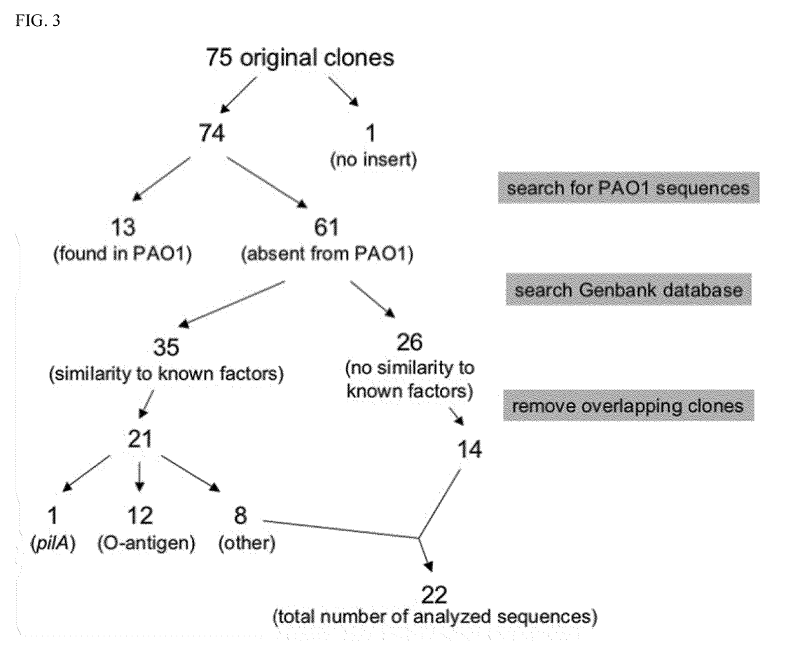

Comparison of a highly virulent strain and a less virulent strain of P. aeruginosa using subtractive hybridization. Since PSE9 exhibited elevated levels of virulence in both the animal and plant models, it was reasoned that this strain had a high likelihood of containing a number of interesting genomic islands encoding virulence factors. For this reason, a PCR-based subtractive hybridization approach was used to identify genetic regions present in PSE9 but absent in the less virulent PAO1 strain. PAO1 was chosen as the reference strain for these experiments because of its relatively low virulence, the availability of its genomic sequence (Stover et al., 2000), and its growth rate, which was equivalent to that of PSE9 in LB medium (data not shown). A subtractive hybridization library consisting of 75 fragments of PSE9 DNA was generated, cloned, sequenced, and compared to the GenBank database (FIG. 3). One clone was found to have no insert and was removed from the library. Of the remaining 74 subtractive hybridization products, 13 (18%) were found to be nearly identical to PAO1 sequences, indicating that they were false positives. Of the 61 sequences that were not present in the PAO1 genome, 35 were similar to known sequences, whereas 26 had no significant similarity to known sequences. Based on sequence alignments, 26 sequences were determined to overlap and were therefore removed from the analysis, leaving 21 products with similarity to known sequences and 14 products without similarity to known sequences.

Of the 21 subtractive hybridization products with similarity to known sequences, 13 were nearly identical to previously sequenced P. aeruginosa genomic islands (FIG. 3 and Table 1). Twelve of these sequences were sequences from a putative serotype 01 O-antigen biosynthesis gene cluster. This cluster is 1 of 11 distinct genetic elements found in P. aeruginosa that encode the biosynthetic enzymes necessary for serotype specificity (Raymond et al., 2002). Thus, PSE9 is related to serotype 01 strains. Strain PAO1, on the other hand, carries the serotype 05 gene cluster, which is divergent from the serotype 01 cluster, explaining why the O-antigen biosynthesis gene cluster sequences were detected by subtractive hybridization. A single insert was similar to a region of the pilA gene, which encodes the pilin subunit of P. aeruginosa type IV pili (Sastry et al., 1985). The type IV pilin genes from different strains of P. aeruginosa segregate into five subclusters that are dispersed among the type IV pilin genes of gram-negative bacteria; pilA genes from different subclusters share less than 30% nucleotide identity (Spangenberg et al., 1997). For these reasons, it has been postulated that the pilA gene of P. aeruginosa was acquired by horizontal transfer. Thus, the identification of the O-antigen biosynthetic cluster and the pilA gene. validated the ability of the subtractive hybridization approach to identify genetic elements that were different in different P. aeruginosa strains. Since the O-antigen biosynthetic cluster and the pilA gene have been characterized previously (Raymond et al., 2002; Spangenberg et al., 1997), they were not further evaluated.

The eight remaining sequences that were similar to known genes had characteristics that suggested that they were parts of novel genomic islands (Table 1). One sequence was similar to an ORF found in CTX, a cytotoxin-converting phage previously isolated from P. aeruginosa strain PA158 (Hayashi et al. (1990); and Nakayama et al. (1999)). This subtractive hybridization product, however, also contained a novel sequence, suggesting that it was from a related but distinct phage. Another sequence had similarity to P. aeruginosa pathogenicity island 1 (PAPI-1) (He et al. (2004)). Note that neither the CTX phage nor PAPI-1 is present in PAO1. The remaining sequences were similar to sequences encoding site-specific recombinases, a zinc-binding transcriptional regulator, a putative phage-related DNA binding protein, and Rhs family elements.

Identification of novel genomic islands. In several cases, the subtractive hybridization products appeared to have identified a small portion of a larger genomic island. Therefore, these sequences were used as tags to identify and characterize the entire genomic island, as well as the DNA flanking the island. To accomplish this, a genomic library of strain PSE9 was constructed using the fosmid vector pSB100, and PCR primers designed to amplify subtractive hybridization products were used to screen the library for individual fosmid clones that contained these sequences (see Materials and Methods). Using this approach, the fosmid library was screened for the presence of the 14 subtractive hybridization products with no similarity to known genes, as well as the eight sequences with similarity to genes not found in PAO1 (FIG. 3). Of these 22 sequences, 20 were represented in the library. The two sequences that were not detected were a clone with similarity to an Rhs family gene and a clone with similarity to PAPI-1. Overall, 25 fosmid clones that contained at least 1 of the 20 subtractive hybridization sequences were identified. A subset of nine fosmid clones cumulatively contained all 20 of the subtractive hybridization sequences and was used in subsequent analyses.

Sequencing of a large PSE9 genomic island. To further characterize the PSE9-associated genomic islands, the complete nucleotide sequence of the subset of nine fosmid clones containing all 20 subtractive hybridization products was obtained. Overall, this analysis suggested that the set of fosmid inserts analyzed represented seven distinct genomic islands located at different sites in the P. aeruginosa genome (data not shown). Here, the largest of these novel islands is characterized.

The inserts of three fosmids were determined to contain portions of a single large genomic island with similarity to PAPI-1. The complete sequence of this island was obtained. Since this island differed substantially from PAPI-1 (see below), it was given a unique name. Using the nomenclature system of Liang et al. (2001), Larbig et al. (2002), and Klockgether et al. (2004), who identified PAGI-1, PAGI-2, PAGI-3, and PAGI-4, this large island was designated “PAGI-5.”

PAGI-5 is the largest of the genomic islands identified in PSE9; it is 99,276 bp long. Its G+C content is 59.6%, which is lower than the PAO1 overall genome G+C content, 66.6% (Stover et al. (2000)). This island is predicted to contain 121 ORFs and is integrated into the genome immediately adjacent to a tRNALys gene (PA0976.1) at bp 1,061,197 in the core chromosome. (PAO1 gene designations are used throughout this paper (Stover et al. (2000).) tRNA genes frequently serve as integration sites for prokaryotic genomic islands (Williams 2002), and this P. aeruginosa tRNA gene is no exception. It serves as the insertion site for PAPI-1, PAPI-2, pKLK106, and PAGI-4 (Kiewitz et al. (2000); Klockgether et al. (2004); and Qui et al. (2006)).

Based on sequence comparisons, PAGI-5 is related to a known family of P. aeruginosa genomic islands that includes PAPI-1, PAPI-2, ExoU islands A, B, and C, and an unnamed 8.9-kb tRNALys-associated island in strain PAO1 (He et al. (2004); Klockgether et al. (2004); and Kulasekara et al. (2006)). These islands themselves comprise a subset of a large family of pKLC102-related genomic islands prevalent in beta- and gammaproteobacteria (Klockgether et al. (2007)). The members of the pKLC102 family of genomic islands are plasmid-phage hybrids that consist of two parts: a relatively conserved core set of genes involved in propagation, replication, and partitioning, and variable “cargo” gene cassettes (Klockgether et al. (200); Klockgether et al. (2007); and Wurdemann et al. (2007)). Kulasekara et al., (2006) proposed that the PAPI-1-related islands evolved from an ancestral integrative plasmid similar to pKLC102. According to their model, during evolution these related elements diverged into two clades, which can be distinguished by the presence of the genes encoding the type III effector protein ExoU and its chaperone SpcU. In one clade, consisting of PAPI-2 and ExoU islands A, B, and C, the exoU and spcU genes are present, but additional rearrangements during or following integration led to loss of the partitioning factor gene of the pKLC102-like plasmid (Kulasekara et al. (2006)). The loss of this plasmid feature may have fixed the island into the chromosome (Kulasekara et al. (2006)). Consistent with this model is the finding that each of these islands is integrated into the same tRNALys gene (PA0976.1). The second clade consists of PAPI-1 and an 8.9-kb tRNALys-associated island of strain PAO1, which evolved from a lineage of the ancestral plasmid that did not acquire (or lost) the exoU and spcU genes. PAPI-1 has maintained the features of the integrated plasmid and has been shown to be transferable (Qiu et al. (2006)). As a result, PAPI-1 can integrate into either of the two tRNALys genes (PA4541.1 and PA0976.1) present in the P. aeruginosa genome (Qiu et al. (2006)). It is not surprising that some members of this clade can integrate into either of these sites, since the pKLC102-like plasmid pKLK106 has been shown to integrate into either site (Kiewitz et al. (2000); and Klockgether et al. (2004)). PAGI-5 appears to be another member of the second clade, since it also does not contain the exoU and spcU genes. PAGI-5 is integrated into the tRNALys gene PA0976.1 in PSE9; additional studies are necessary to determine whether this island is also transferable and can be found in the tRNALys gene PA4541.1 in other strains. PCR analysis indicated that the integration site in the PA4541.1 tRNALys gene of PSE9 is unoccupied (data not shown). Interestingly, like PAPI-1, PAGI-5 contains an intact partitioning factor gene (5PG121), suggesting that it may be transferable.

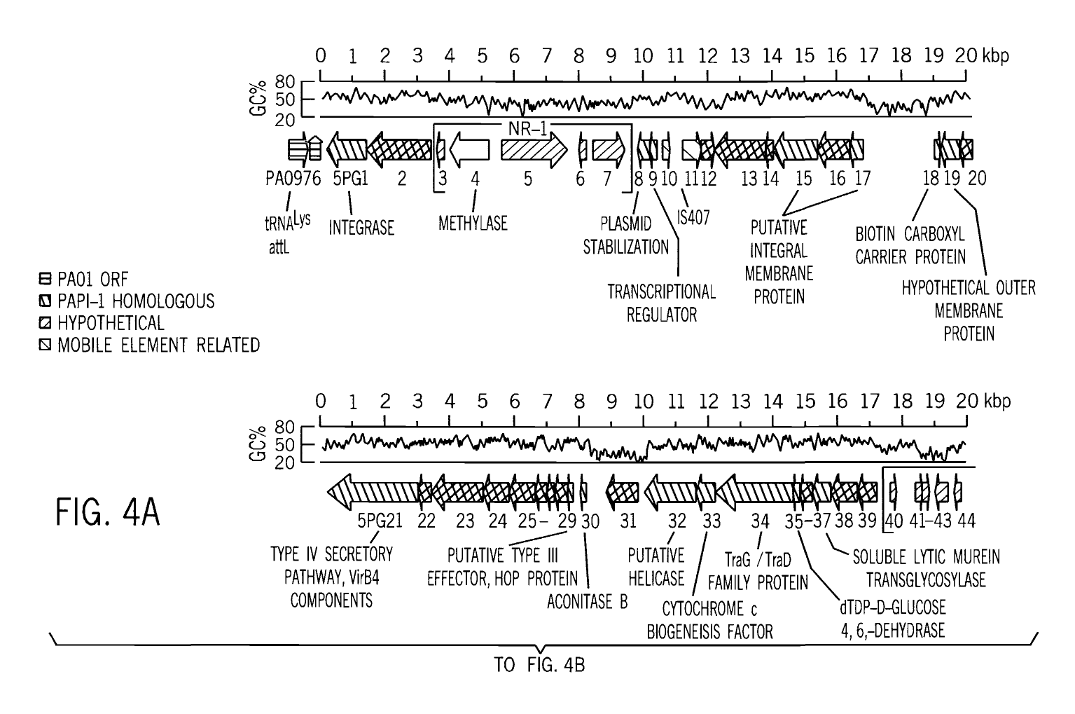

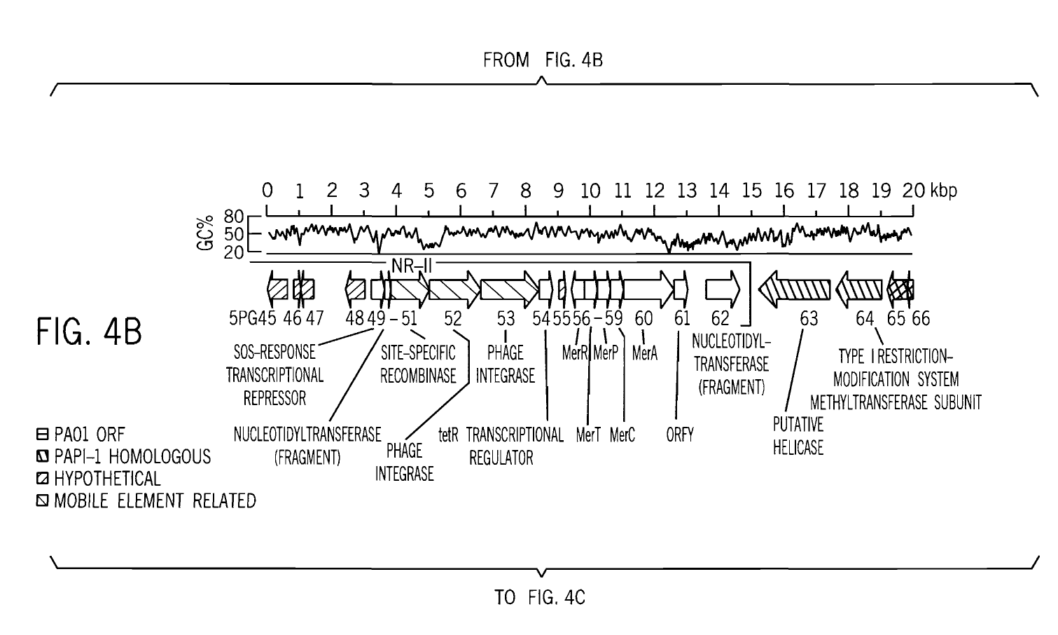

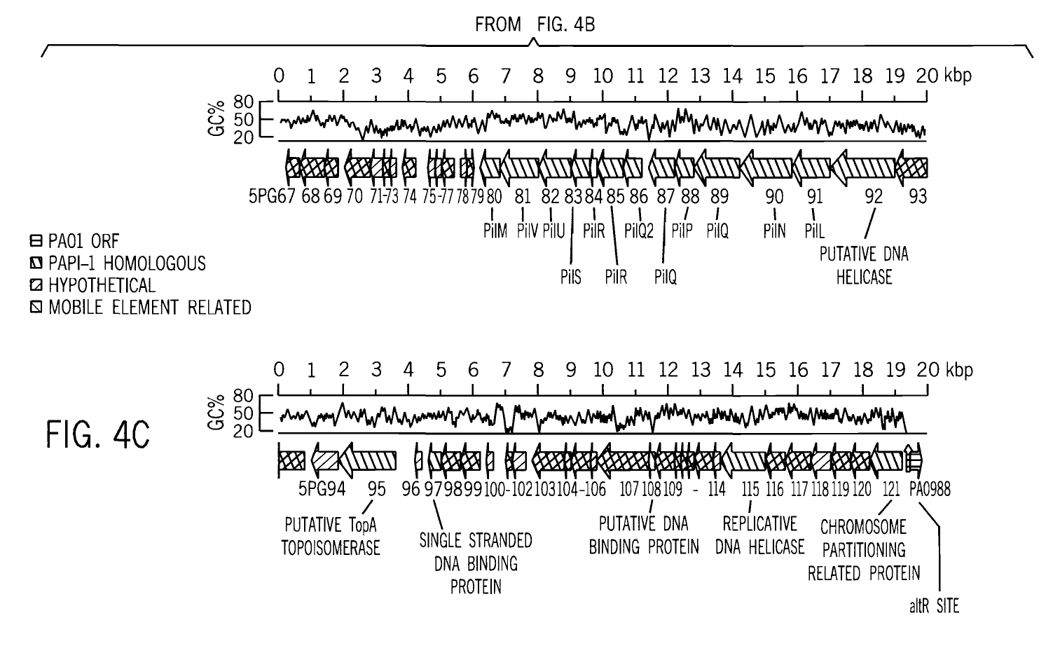

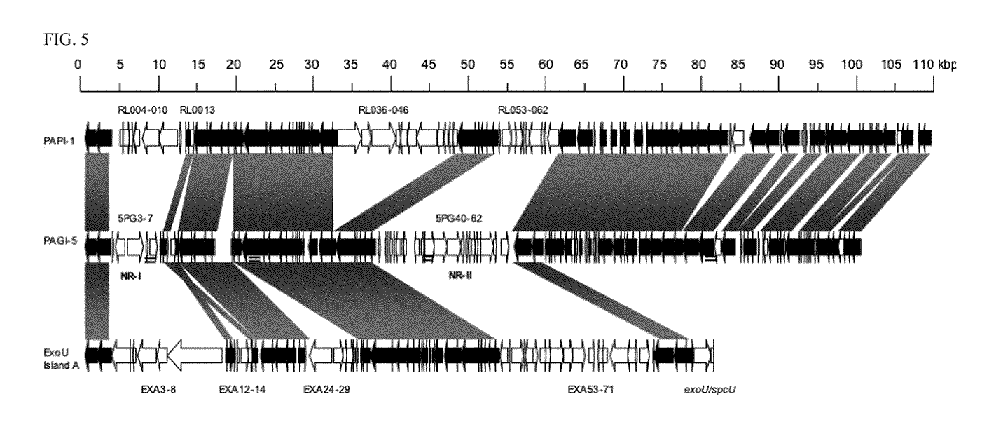

Of this group of related genomic islands, PAGI-5 is most similar to PAPI-1; 79 of the 121 predicted PAGI-5 ORFs share similarity to PAPI-1 ORFs (FIGS. 4 and 5; see Table 2). Yet PAGI-5 carries a substantial amount of genetic information that is not present in PAPI-1 and also lacks a number of PAPI-1 ORFs. Since PAPI-1 has been thoroughly described previously (He et al. (2004)), the features of PAGI-5 that differ from features of PAPI-1 are highlighted (FIGS. 4 and 5). A 14.8-kb region of PAPI-1 containing 11 ORFs (RL036 to RL046) is not present in PAGI-5. Two other large regions of PAPI-1 are also missing from PAGI-5, but both of these regions are replaced in PAGI-5 with novel DNA sequences. A 6.2-kb region containing seven PAPI-1 ORFs (RL004 to RL010) is replaced by an 8.5-kb sequence, which is referred to as NR-I. NR-I contains five ORFs (5PG3 to 5PG7), four without similarity to previously characterized genes and one (5PG4) that shares 25% identity with a putative methylase gene from Bacillus cereus. This region of PAGI-5 has a G+C content of 50.5%, which is considerably lower than the G+C content of PAGI-5 as a whole (59.6%), suggesting that its origins are distinct from those of the remainder of PAGI-5 (FIG. 4). A central 7.8-kb PAPI-1 region containing ORFs RL053 to RL062 is replaced in PAGI-5 by a 17.9-kb sequence, which is referred to as NR-II. NR-II contains 23 predicted ORFs (5PG40 to 5PG62) and has a G+C content of 56.6%. The first part of this region contains nine predicted ORFs that lack similarity to any previously characterized sequences. These ORFs are followed by an ORF (5PG49) that shares 99% identity with an ORF from P. aeruginosa strain C3719, which is predicted to encode an SOS response transcriptional repressor due to the presence of a region similar to a LexA domain (COG1974) (Fogh et al. (1994)). The 5PG49 and C3719 ORFs both share only 46% similarity and 32% identity overall with the ORF encoding PAO1 LexA (PA3007), the canonical SOS response repressor (Garriga et al. (1992)). The next ORF has apparently served as the integration site for a small genetic element (FIG. 4). This ORF, whose product is similar to a nucleotidyltransferase, is split into two separate ORFs (5PG50 and 5PG62) that flank the putative genetic element. Consistent with this interpretation is the finding that a region similar to 5PG50 and 5PG62 (along with 5PG43 to 5PG49) but lacking the inserted genetic element is present in strain C3719. The element itself consists of 11 ORFs, including ORFs encoding a predicted recombinase and two predicted integrases that are between 28% and 40% identical to the products of three predicted ORFs of Burkholderia multivorans. The presence of multiple ORFs associated with mobile elements suggests that these 11 ORFs had an evolutionarily distinct origin, which is supported by the presence of a region 99% identical to 5PG51 to 5PG61 in P. aeruginosa strain PA7. Also present in this genetic element is an ORF similar to ORFs encoding members of the TetR family of transcriptional regulators, which typically repress gene expression in response to environmental cues (Ramos et al. (2005)). The TetR regulator gene and an adjacent integrase gene are similar to a regulator-integrase gene pair found in P. aeruginosa plasmid pKLC102. In pKLC102 these ORFs are followed by ORFs encoding a putative short-chain dehydrogenase/reductase protein and another TetR regulator protein; in PAGI-5 they are instead adjacent to a cluster of ORFs (5PG56 to 5PG61) similar to a 4-kb fragment of the Pseudomonas mercury resistance transposon Tn5041 (Kholodii et al. (1997); and Larbig et al. (2002)). This cluster includes ORFs encoding homologs of the following proteins: MerR (transcriptional regulator of the CueR family), MerT (mercuric ion transport), MerP (periplasmic mercuric ion binding), MerC [inner membrane Hg(II) uptake], MerA (mercuric ion reductase), and ORFY (hypothetical protein). Thus, PAGI-5 has the potential to confer mercury resistance.

There are other minor differences between PAGI-5 and PAPI-1. For example, in place of RL013 of PAPI-1, PAGI-5 carries IS407, an insertion sequence that contains two ORFs (5PG10 and 5PG11) predicted to encode transposases. Interestingly, all or portions of IS407 are also found in ExoU islands A, B and C, where the sequence is adjacent to the exoU and spcU genes. In contrast, in PAGI-5 and the 8.9-kb genomic island associated with the PAO1 PA0976.1 tRNALys gene, the IS407 sequences are not associated with the exoU and spcU genes, which are not present in these islands. PAPI-1 lacks both IS407 and the exoU and spcU genes (He et al. (2004)). The close association between IS407, this group of genomic islands, and the exoU and spcU genes suggests that this insertion sequence played a role in either the acquisition or loss of the exoU and spcU genes from the ancestor of these elements (Kulasekara et al. (2006)).

Despite these differences, the majority of PAGI-5 is similar to PAPI-1 and even to the less closely related ExoU island A (FIG. 5). For example, ORFs 5PG12 to 5PG39 are conserved in all three islands and appear to involve plasmid-related functions. A subset of these ORFs (5PG21 to 5PG29) is similar to a cluster of genes from PKLC102 (CP73 to CP81) that are conserved in other tRNALys-associated islands in multiple species (Klockgether et al. (2007)). Likewise, ORFs putatively encoding an integrase (5PG1), a plasmid stabilization protein (5PG8), a transcriptional regulator (5PG9), a helicase (5PG63), and a methyltransferase (5PG64) are present in all three islands. These conserved regions are consistent with a common ancestry.

Distribution of PAGI-5 in clinical isolates. As mentioned above, PAGI-5 appears to be a chimeric genomic island consisting of three PAPI-1-related regions and two novel regions (designated NR-I and NR-II) (FIG. 5). To determine the frequency and distribution of these regions among P. aeruginosa strains, a collection of 35 clinical isolates were screened for the for the presence of NR-I, NR-II, and the large PAPI-1-like regions in the center and in the 3′ end of PAGI-5 (FIG. 1B). PCR was used to amplify a sequence within each of these regions in each isolate (FIG. 5). Amplified products from the central and 3′ PAPI-1 conserved regions were observed in 34 (97%) of the 35 clinical isolates (FIG. 1B), consistent with previous reports indicating that PAPI-1-related islands are common among P. aeruginosa strains (Klockgether et al. (2007)). In contrast, amplification of sequences within the novel NR-I and NR-II regions was observed only in highly virulent isolates. Specifically, the NR-I sequence was observed only in PSE9 itself, the most virulent of the 35 isolates, and the NR-II sequence was observed only in seven of the most virulent isolates (FIG. 1B). The presence of NR-II exclusively in highly virulent isolates suggested that it contributed directly to the increased pathogenicity of these isolates or was genetically associated with other factors that contributed.

The novel regions of PAGI-5 encode virulence determinants. To examine the role of NR-I and NR-II of PAGI-5 in virulence, two PSE9 deletion strains were created by homologous recombination (see Materials and Methods). The first mutant strain, PSE9ΔNR-I, had a deletion of bp 3712 to 9342 within NR-I, disrupting or deleting ORFs 5PG4 to 5PG7. The second mutant strain, PSE9ΔNR-II, had a deletion of bp 37,564 to 54,397 of NR-II, disrupting or deleting ORFs 5PG40 to 5PG62. In both strains, the deleted sequences were replaced with gentamicin resistance cassettes. Neither PSE9ΔNR-I nor PSE9ΔNR-II exhibited a growth defect in minimal medium (data not shown).

The importance of NR-I and NR-II to the virulence of PSE9 was then determined using the deletion mutants in the mouse model of acute pneumonia. Mice were inoculated by nasal aspiration with PSE9ΔNR-I, PSE9ΔNR-II, parental strain PSE9, or PAO1, and survival was monitored over the subsequent 7 days. Nearly all mice inoculated with parental strain PSE9 died during the course of the experiment, whereas all of the PAO1-infected mice survived (FIG. 6). The survival curve for mice inoculated with PSE9ΔNR-I resembled that for mice inoculated with parental strain PSE9, indicating that NR-I did not have a major effect on the survival of mice in the acute pneumonia model. In contrast, the mice infected with PSE9ΔNR-II had significantly improved survival compared to the mice infected with parental strain PSE9 (P=0.0036). These results indicate that NR-II of PAGI-5 contributes to the highly virulent phenotype of PSE9.

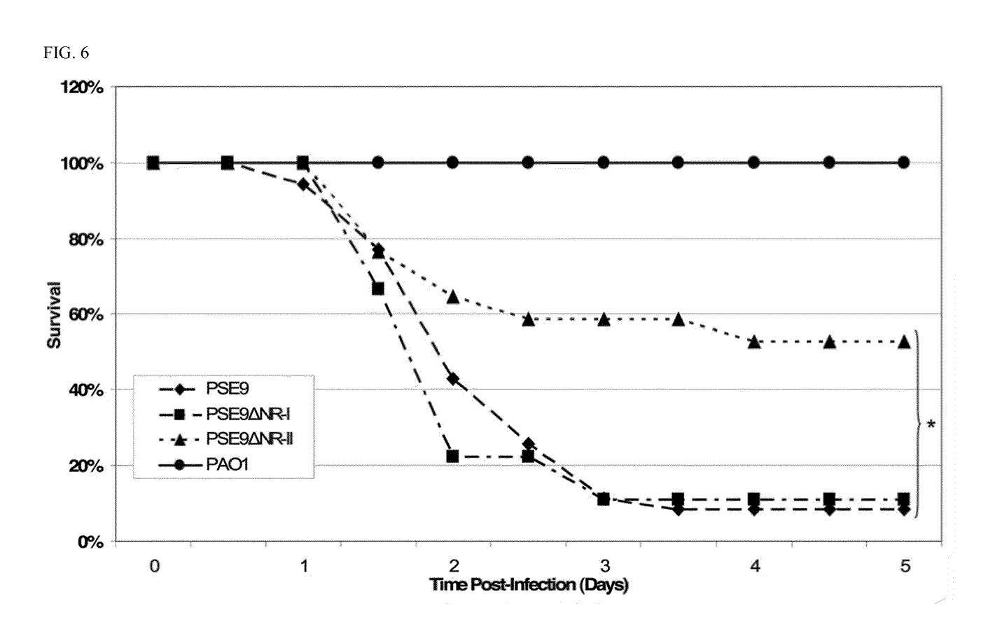

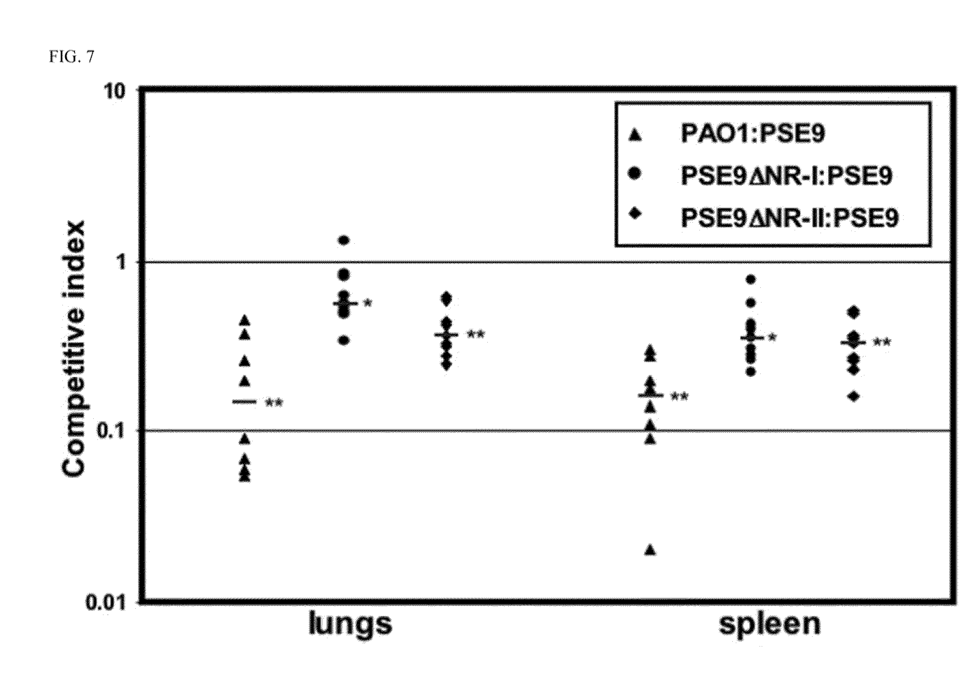

Next, the virulence of the NR-I and NR-II mutants was measured using competition assays, which can detect small differences in virulence between two strains. Mice were inoculated by nasal aspiration with a mixed dose of PSE9ΔNR-I and parental strain PSE9 or with a mixed dose of PSE9ΔNR-II and parental strain PSE9, and the amounts of viable bacteria present in the lungs and spleen were determined after 22 h of infection. Deletion of either NR-I or NR-II resulted in modest but statistically significant decreases in competitive fitness; the mean CIs were 0.56 and 0.37 in the lungs and 0.35 and 0.33 in the spleens, respectively (FIG. 7). In comparison, wild-type strain PAO1 had mean CIs of 0.15 in the lungs and 0.16 in the spleens when it competed against PSE9 (FIG. 7). The finding that there was a substantial difference in virulence between PSE9ΔNR-I and PSE9ΔNR-II in survival assays but there was only a small difference in competition assays may reflect a threshold below which virulence is undetectable in survival assays but apparent in competition assays. Alternatively, the true virulence defect of PSE9ΔNR-II may be masked in competition assays by the “complementing” effect of coinoculated parental strain PSE9. Together with the results of the survival experiments, these results indicate that NR-II of PAGI-5 makes a substantial contribution to the virulence of PSE9, whereas NR-I makes a more modest contribution.

The NR-I and NR-II mutants were also tested using the lettuce leaf model. After 4 days, no difference in either the area of tissue damage or bacterial survival was detected between parental strain PSE9 and either of the mutants (data not shown). Thus, factors other than PAGI-5 NR-I and NR-II must contribute to the virulent phenotype of PSE9 in the lettuce leaf model.

Synopsis. The approach of targeting a highly virulent strain as a source of novel pathogenicity islands in P. aeruginosa has led to identification of seven novel genomic islands, at least one of which is a pathogenicity island. PAGI-5 is a 99-kb hybrid island that is related to the PAPI-1 family of islands but has two large regions with novel sequences, NR-I and NR-II. Deletion of NR-II resulted in a marked decrease in the virulence of parental strain PSE9, and deletion of NR-I resulted in a modest decrease in virulence. Thus, both these regions encode novel virulence determinants that enhance the pathogenicity of PSE9 and are examples of factors responsible for strain-to-strain variation in P. aeruginosa virulence. Examination of other highly virulent strains may lead to identification of additional novel pathogenicity islands in P. aeruginosa, as well as in other bacteria. The advent of relatively inexpensive whole-genome sequencing should greatly facilitate these studies and enable more complete identification of the full arsenal of virulence factors available for use by P. aeruginosa.

### Example 2

Reference is made to the scientific article Battle et al., “Genomic Islands of Pseudomonas aeruginosa,” FEMS Microbiol. Lett. 2009 January; 290(1):70-8. Epub 2008 Nov. 18, the content of which is incorporated herein by reference in its entirety.

### SUMMARY

Key to Pseudomonas aeruginosa's ability to thrive in a diversity of niches is the presence of numerous genomic islands that confer adaptive traits upon individual strains. We reasoned that P. aeruginosa strains capable of surviving in the harsh environments of multiple hosts would therefore represent rich sources of genomic islands. To this end, a strain, PSE9, was identified that was virulent in both animals and plants. Subtractive hybridization was used to compare the genome of PSE9 with the less virulent strain PAO1. Nine genomic islands were identified in PSE9 that were absent in PAO1; seven of these had not been described previously. One of these seven islands, designated P. aeruginosa genomic island (PAGI)-5, has already been shown to carry numerous interesting ORFs, including several required for virulence in mammals. Here, the remaining six genomic islands, PAGI-6, -7, -8, -9, -10, and -11, which include a prophage element and two Rhs elements, are characterized.

Materials and Methods

Construction and screening of a PSE9 fosmid library. Construction of the fosmid library of PSE9 genomic DNA has been described previously (Battle et al., 2008). The complete library was stored in ten 96-well plates. These plates were screened for the presence of subtractive hybridization sequences by PCR amplification using primers corresponding to the sequences (Table 10). A three-tiered screening method was used, as described previously (Battle et al., 2008).

Sequencing of fosmids. Inserts in fosmids identified as containing subtractive hybridization products were sequenced using the EZ□TN <KAN-2> transposon-mediated sequencing approach (Epicentre) as described previously (Battle et al., 2008).

Sequence assembly, annotation, and analysis. Vector NTI Contig Express (inforMax Inc., Frederick, Md.) was used to assemble contiguous sequences. ORFs were identified using GENDB (Meyer et al., 2003) and GENEMARK (Lukashin & Borodovsky, 1998). The G+C content was calculated by a Vector NTI BIOPLOT (InforMax Inc.) from a sliding 100 bp window. BLASTN and BLASTP were used to identify nucleotide and amino acid sequence similarity, respectively (Altschul et al., 1990). Vector NTI ALIGNX (Informax Inc.) was used to align sequences. The dense alignment surface transmembrane prediction method was used to identify potential transmembrane domains (Cserzo et al., 1997). Primers used to identify PAGI sequences in PSE strains are shown in Table 11.

Nucleotide sequence accession number. The sequences of PAGI-6, -7, -8, -9, -10, and -11 have been submitted to the National Center for Biotechnology Information (NCBI) gene bank under the accession numbers EF611302, EF611303, EF611304, EF611305, EF611306, and EF611307, respectively.

Results and Discussion

Identification of fosmids containing PSE9 genomic islands. As mentioned, subtractive hybridization of PSE9 with PAO1 had yielded 22 PSE9 sequences that did not correspond to characterized PAGIs (Battle et al., 2008). Three of these sequences were used to identify PAGI-5 (Battle et al., 2008). The remaining 19 sequences were used to screen a fosmid library of PSE9 genomic DNA to identify fosmids that contained these sequences. The library was screened as pools using primers designed to amplify subtractive hybridization sequences by PCR. One of the sequences was not present in the library, but the remaining 18 sequences were all found between one and five times. A number of different subtractive hybridization sequences were found together on each of several different fosmid clones, suggesting that they were contained within the same genomic island. Overall, 23 fosmid clones that contained at least one of the 18 subtractive hybridization sequences were identified. A subset of seven fosmid clones cumulatively contained all 18 of the subtractive hybridization sequences. This subset of clones was used in subsequent analyses.

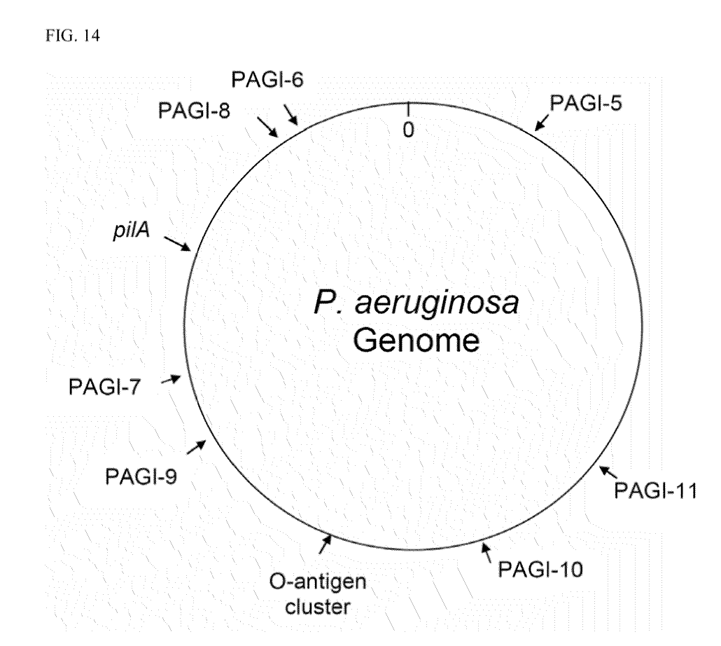

Location of novel genomic islands. The locations of the identified genomic islands within the P. aeruginosa chromosome were determined. Primers were designed to hybridize to the fosmid backbone sequence flanking the insert cloning site to allow sequencing of the ends of each PSE9 genomic insert. Sequencing analysis was then performed. In five of seven fosmids, the PAO1 sequence was found at both ends of the fosmid insert, and the remaining two had PAO1 sequence at one end, allowing placement of the insert in the Pseudomonas aeruginosa core genome. The proximity of the flanking PAO1 sequence found in the latter two fosmids indicated that they represented opposite ends of a single genomic island. Overall, this analysis suggested that the set of analyzed fosmid inserts represented six distinct genomic islands located at different sites in the Pseudomonas aeruginosa genome (Table 4 and FIG. 14).

Sequencing of PSE9 genomic islands. To further characterize the PSE9 genomic islands, the complete nucleotide sequence of the subset of fosmids containing all 18 subtractive hybridization products was obtained. In cases in which the PSE9 genomic island extended beyond the end of the fosmid insert, PCR primers were designed to amplify a sequence at the border of the insert, and the fosmid library was rescreened for the presence of this sequence. In this way, the complete sequence of each PSE9 genomic island was obtained. Altogether, six distinct genomic islands varying in size from 44 to 2 kb were identified (Table 4). Using the nomenclature system of Liang et al. (2001), Larbig et al. (2002), Klockgether et al. (2004), and Battle et al. (2008) who identified PAGIs -1, -2, -3, -4, and -5, these six novel genomic islands were named PAGI-6, -7, -8, -9, -10, and -11 (Table 4). Each island was in turn annotated.

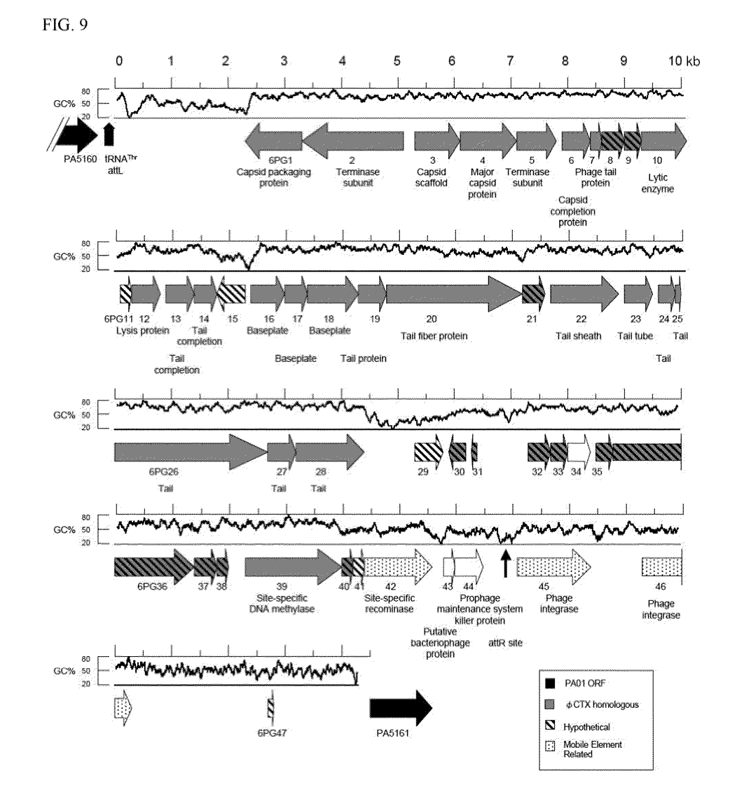

PAGI-6. The first genomic island, PAGI-6, is 44 302 bp in size and has a G+C content of 60.6%, 6% less than that of the overall genome of P. aeruginosa (FIG. 9). It is integrated into a site immediately flanking a tRNA^{Thr} gene (PA5160.1) between genes annotated to encode a drug efflux transporter (PA5160) and a dTDP-D-glucose 4,6-dehydratase (PA5161). [Unless otherwise noted, PAO1 gene designations will be used throughout this discussion (Stover et al., 2000).] PAGI-6 has many large regions highly similar to φCTX, a cytotoxin-converting phage isolated from P. aeruginosa strain PA158 (Nakayama et al., 1999). φCTX is a member of the Pseudomonas aeruginosa R-pyocin-related family of phages (Hayashi et al., 1994). As their name implies, these phages have similarities to R-pyocin-type bacteriocins (Shinomiya & Ina, 1989), bacterially derived proteins with antimicrobial properties (Riley & Wertz, 2002). It has been proposed that R-pyocins are defective phages that have been evolutionarily selected to function as bacteriocins (Nakayama et al., 2000). Thus, PAGI-6 appears to be or to have evolved from a prophage. Interestingly, PAGI-6 and φCTX have different chromosomal locations; in PSE9, PAGI-6 is integrated into the tRNA^{Thr} gene PA5160.1, but in Pseudomonas aeruginosa strain PA158 the φCTX phage genome is integrated into tRNA^{Ser} gene PA2603.1 (Hayashi et al., 1993), neither of which were previously identified as RGPs (Mathee et al., 2008).

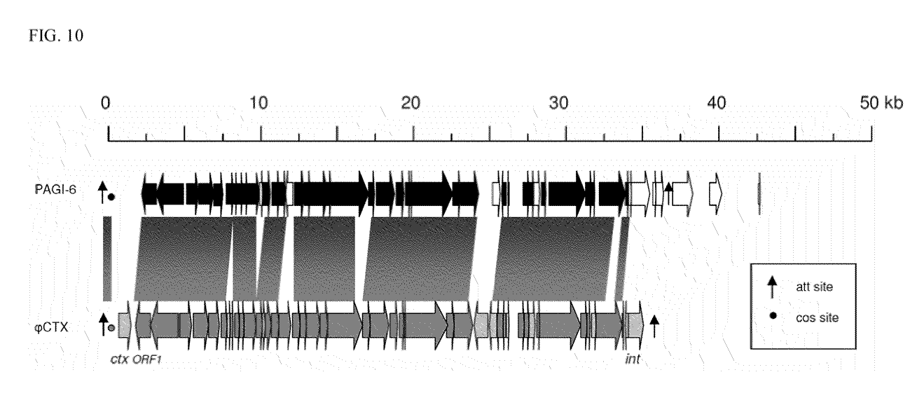

PAGI-6 is somewhat larger than the genome of φCTX, which is 35 538 bp in size, but both elements are relatively conserved over the majority of their sequences (FIG. 10). Notable differences include the absence of φCTX cytotoxin and φCTX integrase genes (ctx and int) from PAGI-6, and the presence of the 7403 bp segment of DNA that follows the attR site of PAGI-6 containing two predicted integrase genes and a stretch of DNA devoid of ORFs, except for a small 99 bp predicted ORF.

In both PAGI-6 and φCTX, sequences from 2,345 bp to 34,086 bp encode a number of putative phage-related structural and enzymatic proteins (FIGS. 9 and 10, Table 6). Despite this overall similarity, there are some interesting differences between these two elements (FIGS. 9 and 10). Like the φCTX genome, PAGI-6 is predicted to contain 47 ORFs, but only 36 of the PAGI-6 ORFs are similar to φCTX ORFs. The remaining 11 φCTX ORFs are not present in PAGI-6; these include φCTX ORFs 7, 12.5, 15, 28, 29, 32, 35, 40, 43, as well as the integrase-encoding gene (int) and, importantly, the cytotoxin gene itself (ctx). Nakayama and colleagues have postulated that some of these ORFs (e.g. ORF 15, 28, 29, and ctx) were not part of the ancestral φCTX genome (Nakayama, et al., 1999). Their absence from PAGI-6 further supports this supposition.

In the φCTX genome, ctx is found at the beginning of the element, between the cohesive end (cos) site and ORF1 (FIG. 10). Whereas the φCTX genome as a whole has a G+C content of 62.6%, the ctx gene has a lower G+C content (53.8%), suggesting its origin is distinct from that of the remainder of φCTX (Hayashi, et al., 1993, Nakayama, et al., 1999). Consistent with this interpretation is that several other members of the φCTX phage family lack the ctx gene (Nakayama, et al., 1999). Likewise, in PAGI-6 the car gene is replaced by 1589 bp fragment of DNA that does not contain an apparent ORF and shares no similarity with the ctx gene. This fragment does, however, contain two pseudogenes that are similar to ORFs in a prophage-related genomic island from Pseudomonas syringae pathovar (pv). phaseolicola. This 1589 bp sequence has a G+C content of 46.7%, significantly lower than the 60.6% G+C content of PAGI-6 (FIG. 9), suggesting its origin is also distinct from that of the rest of the island.

On the other end of PAGI-6, the φCTX integrase gene (int) has been replaced by a 2,768 bp piece of DNA containing multiple ORFs (FIGS. 9 and 10). This DNA fragment contains a putative recombinase gene (6PG42) that is 70% identical to a phage-associated integrase gene (PSPPH_{—}4973) found in the P. syringae prophage PSPPH06. The PAGI-6 recombinase gene is flanked on one side by two ORFs (6PG43 and 6PG44) that share 50% identity to putative bacteriophage protein-encoding genes from Salmonella enterica. The function of the first is unclear, but the second is predicted to encode a protein with homology to the prophage maintenance system killer protein Doc. Doc has been described in the P1 bacteriophage of Escherichia coli and helps in phage maintenance by causing lethality in cells that have been cured of the phage (Lehnherr, et al., 1993). These ORFs are followed by a 46 bp duplication of the 3′ end of the tRNA^{Thr} gene, which likely represents the duplication of the attachment site that occurred during integration (attR). Instead of marking the end of the island, however, the attR site is followed by an additional 7,403 bp of DNA that contains two additional predicted integrase genes (6PG45 and 6PG46) and a stretch of DNA devoid of ORFs with the exception of one small 99 bp sequence (6PG47). A 3700 bp stretch of DNA carrying the two integrase genes is 90% similar to DNA blocks found in strains 2192 and PACS2. This flanking segment of DNA may represent the remnants of a second genetic element. Composite genomic islands can be formed when a second mobile element integrates into an attL or attR site of an already integrated distinct genomic element (Qiu, et al., 2006). Despite the absence of an additional attR site at the very end of the island, the presence of integrase ORFs beyond the attR site, as well as the differing G+C content of the regions before and after the attR site (62.5% versus 51.3%) supports this composite element hypothesis.

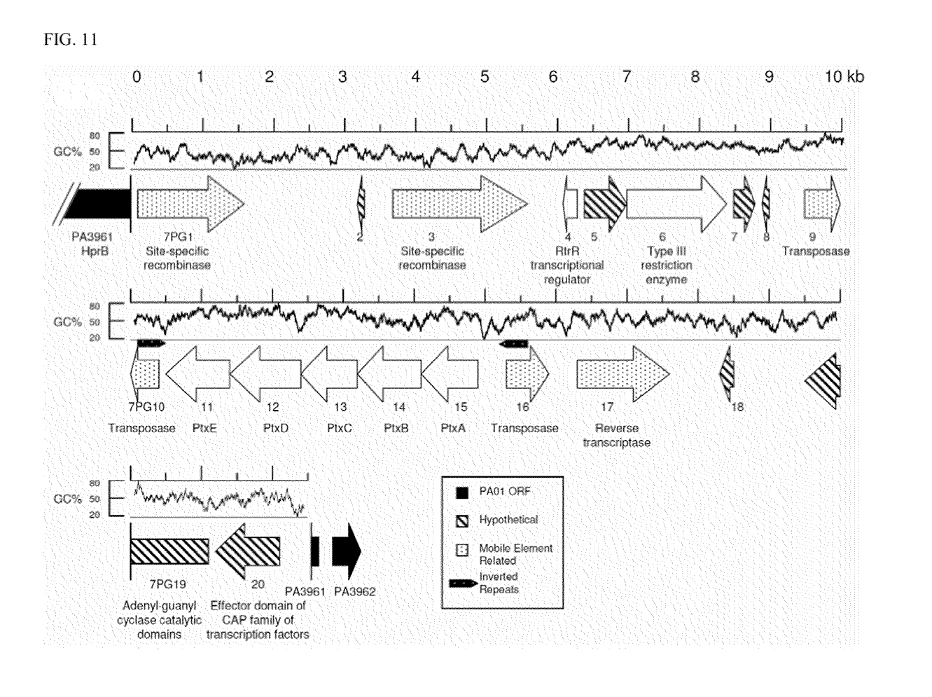

PAGI-7. The next largest identified island was PAGI-7 (Fleiszig et al. (1997)). This island is 22 479 bp in size and has a G+C content of 55.8%. PAGI-7 is not found within a tRNA gene, but instead is integrated within PAO1 ORF PA3961, which is predicted to encode HprB, a probable ATP-dependent helicase that is also not a previously identified RGP. Although the island interrupts PA3961, no portion of this ORF is deleted or repeated. PAGI-7 contains 20 ORFs (FIG. 11, Table 7), including multiple mobility-associated ORFs, predicted transcriptional regulators, and a predicted ptxABCDE operon. The latter was first identified in Pseudomonas stutzeri, where it is required for the oxidation of phosphite to phosphate (Metcalf & Wolfe, 1998; Costas et al., 2001).

PAGI-7 contains 20 total ORFs (FIG. 11, Table 7). One ORF (7PG4) shares 87% identity with an RtrR transcriptional regulator found in the fox genomic island from Pseudomonas syringae pv. actinidiae, a pathogenicity island that encodes a toxin causing chlorosis (discoloration due to lack of chlorophyll) in plants by inhibiting an enzyme in the urea cycle (Mitchell & Bieleski, 1977, Genka, et al., 2006). Seven ORFs (7PG2, 5, 7, 8, 18-20) are either not similar to characterized genes or have similarity only to ORFs encoding hypothetical proteins from Pseudomonas aeruginosa, Shewenella aquaeolei or Caulobacter crescentus. One of the C. crescentus similar ORFs (7PG19) contains two adenyl-guanylyl cyclase domains (75% and 85% alignment) (Liu, et al., 1997) and the other ORF (7PG20) has 90% alignment to the effector domain of the CAP family of transcription factors (McKay & Steitz, 1981), of which cAMP receptor protein is a member. Thus these genes may encode proteins involved cAMP signaling.

PAGI-7 also carries a region (7PG11-15) that shares 99% nucleotide identity to the ptxABCDE operon found in Pseudomonas stutzeri. In P. stutzeri, this operon is required for the oxidation of phosphite to phosphate, with ptxA, B, and C predicted to encode components of a phosphite transporter, ptxD a phosphite dehydrogenase, and ptxE a transcriptional regulator (Metcalf & Wolfe, 1998, Costas, et al., 2001). Expression of the P. stutzeri operon is upregulated under phosphate starvation conditions, suggesting that it could potentially provide an alternate route of phosphorous acquisition by oxidizing phosphite to phosphate, but its actual role in nature is less clear since the environment contains very little phosphite (White & Metcalf, 2004). A 4660 bp DNA segment 98% similar to the PAGI-7 pix operon is also found in strain 2192, but at the 3′ end of a 63 kbp genomic island located next to the PA2729 homolog, or RGP28 (Mathee, et al., 2008). The PAGI-7 ptx operon is flanked by inverted repeats that carry duplicated ORFs encoding putative IS5-related transposases (7PG10 and 7PG16). In addition, it has a G+C content of 61.4%, higher than the overall 55.8% G+C content of PAGI-7, suggesting that this region has an origin distinct from that of the rest of the island.

The seven remaining ORFs of PAGI-7 are similar to genes associated with mobile elements, including genes encoding recombinases (7PG1 and 7PG3), a type III restriction enzyme (7PG6), a putative transposase similar to an ORF found in IS66-related insertion sequences (7PG9), and a reverse transcriptase (7PG17). A 2063 bp region of DNA that spans most of 7PG4-6 is 90% similar to a block of DNA found in strain PA7. The ORF encoding the predicted reverse transcriptase homolog is located within a 1.8 kb region that is similar to a group II intron found on a megaplasmid from Ralstonia eutropha (Schwartz, et al., 2003). Group II introns are RNA retro-elements that are often associated with mobile DNA elements in bacteria (Dai & Zimmerly, 2002). The two recombinase ORFs (7PG1 and 7PG3) are located at the beginning of the island and are 80% and 76% identical to site-specific recombinase genes from Pseudomonas stutzeri A1501 (PST0585 and PST0587, respectively) (Yan, et al., 2008), and are also similar to two recombinase genes in P. syringae pv. tomato (PSPTO4742 and 4744). The PAGI-7 recombinases are adjacent to the tox pathogenicity island RtrR regulator homolog (7PG4), yet neither P. stutzeri nor P. syringae pv. tomato contain the tox pathogenicity island. However, both the PAGI-7 recombinase ORFs and those of P. stutzeri and P. syringae are contained in islands that have integrated into hrpB genes (P. aeruginosa PA3961, P. stutzeri A 1501. PST0583, and P. syringae pv. tomato PSPTO4745, respectively). This suggests that these recombinases may mediate integration into a conserved site in or near hprB genes. Although less common than integration into tRNA genes, insertion into non-tRNA genes has been observed with other P. aeruginosa islands, such as PAGI-1 (Liang, et al., 2001). Thus PAGI-7 appears to be a large mobile element that acquired the pix operon as well as a group II intron.

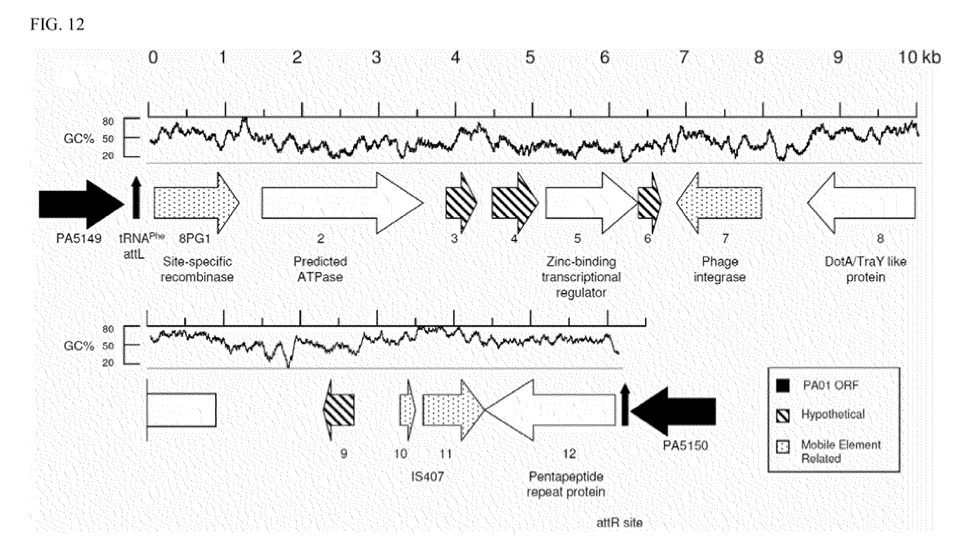

PAGI-8. The next largest identified island was PAGI-8 (FIG. 12). This island, which is 16 195 bp in size and has a G+C content of 54.1%, is inserted into the genome immediately flanking a tRNA^{Phe} gene (PA5149.1) at a site designated as RGP60 by Mathee et al. (2008). A 44 bp duplication of the tRNA gene (representing an attR site) is at the end of the island. PA5149.1 is located between PA5149 and PA5150, which encode a hypothetical protein and a probable short-chain dehydrogenase, respectively. PAGI-8 contains 12 ORFs, including one predicted to encode a protein with 69% identity and 78% similarity to the TraY/DotA-like type IV secretion system protein of Cupriavidus metallidurans strain CH34 (formerly Ralstonia metallidurans), and 19% and 21% identities and 32% and 37% similarities to DotA of Legionella pneumophila and TraY of Escherichia coli, respectively. Also in PAGI-8 are ORFs similar to an ATPase and a zinc-binding transcriptional regulator, but no additional ORFs with similarity to other type IV secretion system genes.

PAGI-8 contains 12 ORFs, several of which may encode proteins with interesting functions (FIG. 12, Table 8). ORF 8PG8 is predicted to encode a protein with 68% identity and 78% similarity to the TraY/DotA-like type IV secretion system protein of Cupriavidus metallidurans strain CH34 (formerly Ralstonia metallidurans), and 19% and 21% identity and 32% and 37% similarity to DotA of Legionella pneumophila and TraY of E. coli, respectively. DotA is an integral cytoplasmic membrane protein thought to comprise part of the Dot/Icm type IV secretion apparatus, although it reportedly is secreted into culture supernatants by the same transport system (Roy & Isberg, 1997, Nagai & Roy, 200.1). Like DotA, the putative protein encoded by 8PG8 is predicted to contain eight membrane-spanning regions, suggesting that it is a transmembrane protein. Type IV secretion systems often play important roles in the propagation of genomic islands (Juhas, et al., 2007), although none of the other ORFs in PAGI-8 are similar to known type IV secretion system genes, so the function of this putative TraY/DotA-like protein in P. aeruginosa is unclear. A second ORF in the island, 8PG5, is predicted to encode a zinc-binding transcriptional regulator with a helix-turn-helix DNA binding motif and a zinc peptidase domain. This predicted transcriptional regulator and its flanking hypothetical ORF (8PG4) are similar to two adjacent ORFs in Photobacterium profundum. Another PAGI-8 ORF, 8PG2, has 22% identity to an ORF predicted to encode a member of the AAA+ superfamily of adenosine triphosphatases (ATPases), and contains a nucleotide binding Walker A motif (Walker, et al., 1982). AAA+ ATPases provide energy for many cellular processes, including some bacterial secretion systems (Akeda & Galan, 2005). Another ORF, 8PG12, has a low level of similarity (15% identity and 20% similarity) to a pentapeptide repeat protein. The function of these proteins is unknown, but they are defined by tandem repeats of a five amino acid motif (Vetting, et al., 2006), yet 8PG12 has only a single 5 amino acid sequence that matches this motif. Four other ORFs in this island (8PG3, 8PG4, 8PG6, 8PG9) are not similar to known genes. PAGI-8 contains 4 ORFs associated with mobile elements: two ORFs related to the transposon IS407 (8PG10 and 8PG11), an ORF predicted to encode a phage integrase (8PG7), and an ORF predicted to encode a site-specific recombinase (8PG 1). Interestingly, the transposon-related genes are 99% identical to the IS407 transposase ORFs PA0986 and PA0987 found in several other Pseudomonas aeruginosa strains, including PAO1 (the 8.9 kb tRNA^{Lys} associated genomic island), C3719, PA14, and PA7 (Stover, et al., 2000). These genes are also found in PAGI-5 of PSE9 and in the ExoU genomic islands (Kulasekara, et al., 2006, Battle, et al., 2008).

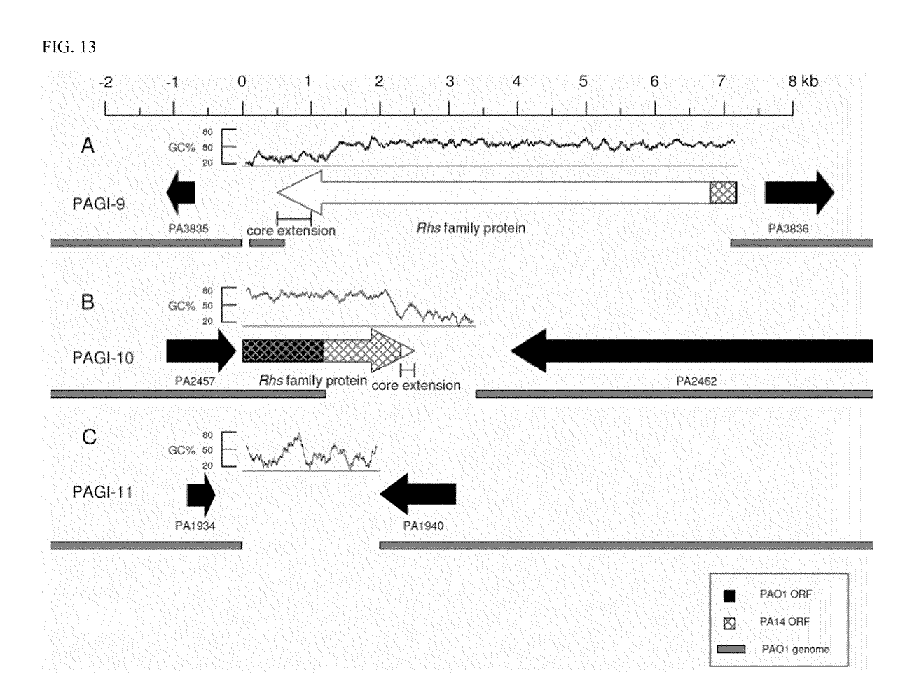

PAGI-9 and PAGI-10. The PAGI-9 island is 6581 bp in size and is located in an intergenic region between PA3835 and PA3836, both of which encode hypothetical proteins (FIG. 13A). This region was not identified as an RGP by Mathee et al. (2008). It has a G+C content of 63.4%. This island consists of a single very large ORF of 6672 bp that is similar to the rearrangement hot spot (Rhs) family of genetic elements (FIG. 13A and Table 9) (Hill, 1999). PAGI-10 is 2194 bp in size and has a G+C content of 56.6%. It is located in RGP25 between PA2457 and PA2462 of PAO1, which encode a hypothetical protein with partial similarity to an Rhs core protein and an extremely large hypothetical protein (5628 amino acids) with a low level of similarity to hemagglutinin, respectively (FIG. 13B). PAGI-10 replaces the PAO1 ORFs PA2458-61. PA2458 has partial similarity to an Rhs element, and PA2459-61 do not have similarity to known genes. Similar to PAGI-9, PAGI-10 contains a single 2457-bp ORF with similarity to an Rhs core ORF.

Both PAGI-9 and PAGI-10 are similar to Rhs elements. These intriguing elements were first characterized in E. coli but were subsequently found in a number of Gram-negative bacteria, including Salmonella, Yersinia, Actinobacillus, Burkholderia, Vibrio, as well as Pseudomonas aeruginosa (Hill, 1999, Mena & Chen, 2007). Their presence and number vary from strain to strain; whereas E. coli strain K-12 contains five Rhs elements that constitute 0.8% of its entire genome (Hill, et al., 1994), other E. coli strains do not harbor any of these elements (Hill, et al., 1995). Their function is unknown, although it has been speculated that they encode proteins that are secreted or associated with the cell wall and that bind to ligands (Hill, et al., 1994). In any case, the maintenance of such large ORFs indicates that Rhs elements are under strong positive selection (Petersen, et al., 2007). Rhs elements vary in structure but typically consist of several of the following components (Hill, 1999): (i) A large Rhs core ORF comprised of a conserved Rhs core followed by a shorter highly variable core extension region (Feulner, et al., 1990). Interestingly although they form a single ORF, the cure and core extension often differ significantly in G+C content, suggesting that the Rhs core ORF is a composite element. A conspicuous feature of the predicted core protein is a repeated peptide motif consisting of YDxxGRL(I/T) (Hill, et al., 1994). (ii) A small downstream ORF that appears to encode a protein with a signal peptide. (iii) A downstream insertion sequence. (iv) An upstream gene encoding a Val-Gly dipeptide repetition (Vgr) protein. Vgr proteins have attracted much interest recently because they have been shown to be virulence determinants associated with novel type VI secretion systems in P. aeruginosa and V. cholerae (Wilderman, et al., 2001, Sheahan, et al., 2004, Mougous, et al., 2006). In the latter bacterium, Vgr proteins are secreted and cause intoxication of mammalian cells upon cell contact (Wildermian, et al., 2001). (v) A gene encoding a hemolysin co-regulated protein (Hcp) (Wang, et al., 1998). Secretion of Hcp by a type VI secretion system has been demonstrated in V. cholerae (Wilderrnan, et al., 2001) and P. aeruginosa (Mougous, et al., 2006).

The PAGI-9 Rhs element does not contain ORFs predicted to encode Vgr or Hcp proteins or an associated insertion sequence. However, its Rhs core ORF does manifest a marked discrepancy in G+C content between the Rhs core itself (64.9%) and the core extension (45.2%) (FIG. 13A). The putative protein encoded by the PAGI-9 Rhs core ORF is nearly identical over the first 2000 amino acids to an Rhs homolog found in P. aeruginosa strain 2192 (PA2G_{—}03278) (Mathee, et al., 2008). However the core extensions of these two ORFs are unrelated. PA14 carries a truncated homolog (the N-terminal 159 amino acids) of this ORF in the same genomic location, followed by 1565 bp of noncoding sequence (Lee, et al., 2006), while PAO1 has 2645 bp of noncoding DNA between PA3835 and PA3836 (Stover, et al., 2000).

Similar to PAGI-9, PAGI-10 contains a single ORF with similarity to an Rhs core ORF. However, whereas the PAGI-9 Rhs core ORF is 6,672 bp, the PAGI-10 ORF is only 2,457 bp. Furthermore, PAGI-10 contains only the 3′portion of this ORF; the 5′ portion is encoded by PAO1 conserved sequence (FIG. 13B). Thus the PAGI-10 genomic insertion extends an ORF already present in the PAO1 genome. PA14 carries two ORFs (PA14 20520 and 32830) that are 97% and 98% identical, respectively, to the PAGI-10 associated ORF over their entire lengths except for the core extensions (Lee, et al., 2006). PA14 32830 is located in the same genomic locus as PAGI-10. Similar ORFs are also found in the recently sequenced strains PACS2, 2192, and C3719 at the same locus; thus PAGI-10 may constitute a locus of the relatively conserved P. aeruginosa genomic backbone that was deleted from PAO1. Over the N-terminal predicted 750 amino acids, the PAGI-10 Rhs core ORF protein is very similar to multiple Rhs-like putative proteins encoded by other P. aeruginosa strains, but the C-terminal region does not share similarity with any of these, suggesting that this ORF encodes a conserved Rhs core protein with a novel core extension. Consistent with this interpretation is the difference in G+C content between the core (64.9%) and the core extension (49.7%) (FIG. 13B). No Vgr- or Hcp-encoding genes or insertion sequences are associated with PAGI-10.

PAGI-11. PAGI-11 is the smallest of the identified genomic islands, consisting of 2003 bp (FIG. 13C). As such, it verifies the power of the subtractive hybridization approach to detect genetic elements as small as 2 kb. PAGI-11 has a G+C content of 50.5% and does not contain any ORFs or repeated sequences. It is located in RGP52 between PA1934 and PA1940 of the PAO1 genome, which encode hypothetical proteins, although a region of PA1940 is similar to a catalase domain. PAGI-11 replaces a 5870-bp segment of the PAO1 genome containing PA1935-1939. PA1935 and PA1936 are predicted to encode proteins of unknown function, and PA1939 is similar to a gene encoding an ATP-dependant endonuclease. PA1937 and PA1938 are similar to two different IS911 ORFs predicted to encode transposases, and are almost completely identical to PA0979 and PA0978, which are part of the 8.9 kb tRNA^{Lys}-associated genomic island of strain PAO1. In addition to PAO1, unique genomic insertions containing between one and five ORFs are found at this locus in strains PA14, PACS2, 2192, and C3719 (Mathee et al., 2008). Thus, it appears that these strains contain mobile genetic elements at the locus where PAGI-11 resides in PSE9.

Distribution of genomic islands in clinical isolates. To determine the frequency and distribution of PAGI-6, -7, -8, -9, and -10 in P. aeruginosa strains, a collection of 35 clinical isolates was screened for sequences found within these genomic islands (Table 5). PCR was used to amplify a sequence from each island in each isolate (Table 11). PAGI-6 was found in two (6%) of the 35 isolates. PAGI-7 and PAGI-9 were both present in the same 16 (46%) isolates. PAGI-8 was found only in strain PSE9 (3%). PAGI-10 was found in 20 (57%) of strains, and with the exception of PSE9, was only present in strains that lacked PAGI-7 and PAGI-9.

### CONCLUSION

In conclusion, the information presented here, along with that previously reported (Battle et al., 2008), demonstrates the utility of targeting a hypervirulent strain of P. aeruginosa as a source of genetic information found in the accessory genome. Applying this approach to a panel of clinical isolates has led to the identification of seven novel genomic islands varying in size from 99 to 2 kb and together containing 201 ORFs. Several are related to known pathogenicity islands, phages, or Rhs elements while others are quite novel. Many of these islands appear to be chimeric in nature, further demonstrating that composite genomic islands occur commonly in the evolution of P. aeruginosa. While three of the seven islands are located in or adjacent to tRNA genes, the remaining four are not, indicating that alternative sites are also capable of being targeted for integration in P. aeruginosa. Together, these results shed additional light on die evolution of genomic islands in P. aeruginosa and attest to the vast amount of genetic information carried by these elements.

### REFERENCES

Akeda Y & Galan J E (2005) Chaperone release and unfolding of substrates in type III secretion. Nature 437: 911-915.
Alibaud, L., T. Kohler, A. Coudray, C. Prigent-Combaret, E. Bergeret, J. Perrin, M. Benghezal, C. Reimmann, Y. Gauthier, C. van Delden, I. Attree, M. O. Fauvarque, and P. Cosson. 2008. Pseudomonas aeruginosa virulence genes identified in a Dictyostelium host model. Cell. Microbiol. 10:729-740.
Altschul, S. F., W. Gish, W. Miller, E. W. Myers, and D. J. Lipman. 1990. Basic local alignment search tool. J. Mol. Biol. 215:403-410.
Battle S E, Meyer F, Rello J, Kung V L & Hauser A R (2008) The hybrid pathogenicity island PAGI-5 contributes to the highly virulent phenotype of a Pseudomonas aeruginosa isolate in mammals. J Bacteriol 190: 7130-7140.
Cheetham, B. F., and M. E. Katz. 1995. A role for bacteriophages in the evolution and transfer of bacterial virulence determinants. Mol. Microbiol. 18:201-208.
Comolli, J. C., A. R. Hauser, L. Waite, C. B. Whitchurch, J. S. Mattick, and J. N. Engel. 1999. Pseudomonas aeruginosa gene products PilT and PiIU are required for cytotoxicity in vitro and virulence in a mouse model of acute pneumonia. Infect. Immun. 67:3625-3630.
Costas A M, White A K & Metcalf W W (2001) Purification and characterization of a novel phosphorus-oxidizing enzyme from Pseudomonas stutzeri WM88. J Biol Chem 276: 17429-17436.
Cserzo M, Wallin E, Simon I, von Heijne G & Elofsson A (1997) Prediction of transmembrane alpha-helices in prokaryotic membrane proteins: the dense alignment surface method. Protein Eng 10: 673-676.
Dai L & Zimmerly S (2002) The dispersal of five group II introns among natural populations of Escherichia coli. Rna 8: 1294-1307.
Dobrindt, U., B. Hochhut, U. Hentschel, and J. Hacker. 2004. Genomic islands in pathogenic and environmental microorganisms. Nat. Rev. Microbiol. 2:414-424.
Dong, X., M. Mindrinos, K. R. Davis, and F. M. Ausubel. 1991. Induction of Arabidopsis defense genes by virulent and avirulent Pseudomonas syringae strains and by a cloned avirulence gene. Plant Cell 3:61-72.
Elrod R & Braun A (1942) Pseudomonas aeruginosa: its role as a plant pathogen. J Bacteriol 44: 633-645.
Feltman, H., G. Schulert, S. Khan, M. Jain, L. Peterson, and A. R. Hauser. 2001. Prevalence of type III secretion genes in clinical and environmental isolates of Pseudomonas aeruginosa. Microbiology 147:2659-2669.
Feulner G, Gray J A, Kirschman J A, et al. (1990) Structure of the rhsA locus from Escherichia coli K-12 and comparison of rhsA with other members of the rhs multigene family. J Bacteriol 172: 446-456.
Finck-Barbancon, V., J. Goranson, L. Zhu, T. Sawa, J. P. Wiener-Kronish, S. M. J. Fleiszig, C. Wu, L. Mende-Mueller, and D. Frank. 1997. ExoU expression by Pseudomonas aeruginosa correlates with acute cytotoxicity and epithelial injury. Mol. Microbiol. 25:547-557.
Finlay B B & Falkow S (1997) Common themes in microbial pathogenicity revisited. Microbiol. Mol Biol R 61: 136-169.
Fitzsimmons, S. C. 1993. The changing epidemiology of cystic fibrosis. J. Pediatr. 122:1-9.
Fleiszig, S. M. J., J. P. Wiener-Kronish, H. Miyazaki, V. Vallas, K. Mostov, D. Kanada, T. Sawa, T. S. B. Yen, and D. Frank. 1997. Pseudomonas aeruginosa-mediated cytotoxicity and invasion correlate with distinct genotypes at the loci encoding exoenzyme S. Infect. Immun. 65:579-586.
Fogh, R. H., G. Ottleben, H. Ruterjans, M. Schnarr, R. Boelens, and R. Kaptein. 1994. Solution structure of the LexA repressor DNA binding domain determined by 1H NMR spectroscopy. EMBO J. 13:3936-3944.
Gaillard, C., and F. Strauss. 1990. Ethanol precipitation of DNA with linear polyacrylamide as carrier. Nucleic Acids Res. 18:378.
Garriga, X., S. Calero, and J. Barbe. 1992. Nucleotide sequence analysis and comparison of the lexA genes from Salmonella typhimurium, Erwinia carotovora, Pseudomonas aeruginosa and Pseudomonas putida. Mol. Gen. Genet. 236:125-134.
Genka H, Baba T, Tsuda M, et al. (2006) Comparative analysis of argK-tox clusters and their flanking regions in phaseolotoxin-producing Pseudomonas syringae pathovars. J Mol Evol 63: 401-414.
Glazebrook, J. S., R. S. Campbell, G. W. Hutchinson, and N. D. Stallman. 1978. Rodent zoonoses in North Queensland: the occurrence and distribution of zoonotic infections in North Queensland rodents. Aust. J. Exp. Biol. Med. Sci. 56:147-156.
Green, S. K., M. N. Schroth, J. J. Cho, S. K. Kominos, and V. B. Vitanza-jack. 1974. Agricultural plants and soil as a reservoir for Pseudomonas aeruginosa. Appl. Microbiol. 28:987-991.
Hammer A S, Pedersen K, Andersen T H, Jorgensen J C & Dietz H H (2003) Comparison of Pseudomonas aeruginosa isolates from mink by serotyping and pulsed-field gel electrophoresis. Vet Microbiol 94: 237-243.
Hauser, A. R., E. Cobb, M. Bodi, D. Mariscal, J. Vallés, J. N. Engel, and J. Rello. 2002. Type III protein secretion is associated with poor clinical outcomes in patients with ventilator-associated pneumonia caused by Pseudomonas aeruginosa. Crit. Care Med. 30:521-528.
Hauser, A. R., P. J. Kang, and J. Engel. 1998. PepA, a novel secreted protein of Pseudomonas aeruginosa, is necessary for cytotoxicity and virulence. Mol. Microbiol. 27:807-818.
Hayashi, T., T. Baba, H. Matsumoto, and Y. Terawaki. 1990. Phage-conversion of cytotoxin production in Pseudomonas aeruginosa. Mol. Microbiol. 4:1703-1709.
Hayashi T, Matsumoto H, Ohnishi M & Terawaki Y (1993) Molecular analysis of a cytotoxin-converting phage, phi CTX, of Pseudomonas aeruginosa: structure of the attP-cos-ctx region and integration into the serine tRNA gene. Mol Microbiol 7: 657-667.
Hayashi T, Matsumoto H, Olnishi M, Yokota S, Shinomiya T, Kageyama M & Terawaki Y (1994) Cytotoxin-converting phages, phi CTX and PS21, are R pyocin-related phages. FEMS Microbiol Lett 122: 239-244.
He, J., R. L. Baldini, E. Deziel, M. Saucier, Q. Zhang, N. T. Liberati, D. Lee, J. Urbach, H. M. Goodman, and L. G. Rahme. 2004. The broad host range pathogen Pseudomonas aeruginosa strain PA14 carries two pathogenicity islands harboring plant and animal virulence genes. Proc. Natl. Acad. Sci. USA 101:2530-2535.
Hill C W (1999) Large genomic sequence repetitions in bacteria: lessons from rRNA operons and Rhs elements. Res Microbiol 150: 665674.
Hill C W, Sandt C H & Vlazny D A (1994) Rhs elements of Escherichia coli: a family of genetic composites each encoding a large mosaic protein. Mol. Microbiol. 12: 865-871.
Hill C W, Feulner G, Brody M S, Zhao S, Sadosky A B & Sandt C H (1995) Correlation of Rhs elements with Escherichia coli population structure. Genetics 141: 15-24.
Hoadley, A. W. 1977. Pseudomonas aeruginosa in surface waters, p. 31-57. In V. M. Young (ed.), Pseudomonas aeruginosa: ecological aspects and patient colonization. Raven Press, New York, N.Y.
Hoang, T. T., A. J. Kutchma, A. Becher, and H. P. Schweizer. 2000. Integration-proficient plasmids for Pseudomonas aeruginosa: site-specific integration and use for engineering of reporter and expression strains. Plasmid 43:59-72.
Hogan, D. A., and R. Kolter. 2002. Pseudomonas-Candida interactions: an ecological role for virulence factors. Science 296:2229-2232.
Holloway, B. W., V. Krishnapillai, and A. F. Morgan. 1979. Chromosomal genetics of Pseudomonas. Microbiol. Rev. 43:73-102.
Jander, G., L. G. Rahme, and F. M. Ausubel. 2000. Positive correlation between virulence of Pseudomonas aeruginosa mutants in mice and insects. J. Bacteriol. 182:3843-3845.
Juhas M, Crook D W, Dimopoulou I D, Lunter G, Harding R M, Ferguson D J & Hood D W (2007) Novel type IV secretion system involved in propagation of genomic islands. J. Bacteriol. 189: 761-771.
Kholodii, G. Y., O. V. Yurieva, Z. Gorlenko, S. Z. Mindlin, I. A. Bass, O. L. Lomovskaya, A. V. Kopteva, and V. G. Nikiforov. 1997. Tn5041: a chimeric mercury resistance transposon closely related to the toluene degradative transposon Tn4651. Microbiology 143:2549-2556.
Kiewitz, C., K. Larbig, J. Klockgether, C. Weinel, and B. Tummler. 2000. Monitoring genome evolution ex vivo: reversible chromosomal integration of a 106 kb plasmid at two tRNALys gene loci in sequential Pseudomonas aeruginosa airway isolates. Microbiology 146:2365-2373.
Klockgether, J., O. Reva, K. Larbig, and B. Tummler. 2004. Sequence analysis of the mobile genome island pKLC102 of Pseudomonas aeruginosa C. J. Bacteriol. 186:518-534.
Klockgether, J., D. Wurdemann 0. Reva, L. Wiehlmann, and B. Tummler. 2007. Diversity of the abundant pKLC102/PAGI-2 family of genomic islands in Pseudomonas aeruginosa. J. Bacteriol. 189:2443-2459.
Kulasekara, B. R., H. D. Kulasekara, M. C. Wolfgang, L. Stevens, D. W. Frank, and S. Lory. 2006. Acquisition and evolution of the exoU locus in Pseudomonas aeruginosa. J. Bacteriol. 188:40374050.
Larbig, K. D., A. Christmann, A. Johann, J. Klockgether, T. Hartsch, R. Merkl, L. Wiehimann, H. J. Fritz, and B. Tummler. 2002. Gene islands integrated into tRNAGly genes confer genome diversity on a Pseudomonas aeruginosa clone. J. Bacteriol. 184:6665-6680.
Lawrence, J. G. 2005. Horizontal and vertical gene transfer: the life history of pathogens. Contrib. Microbiol. 12:255-271.
Lee, D. G., J. M. Urbach, G. Wu, N. T. Liberati, R. L. Feinbaum, S. Miyata, L. T. Diggins, J. He, M. Saucier, E. Deziel, L. Friedman, L. Li, G. Grills, K. Montgomery, R. Kucherlapati, L. G. Rahme, and F. M. Ausubel. 2006. Genomic analysis reveals that Pseudomonas aeruginosa virulence is combinatorial. Genome Biol. 7:R90.
Lehnherr H, Maguin E, Jafri S & Yarmolinsky M B (1993) Plasmid addiction genes of bacteriophage P1: doc, which causes cell death on curing of prophage, and phd, which prevents host death when prophage is retained. J Mol Biol 233: 414-428.
Liang, X., X. Q. Pham, M. V. Olson, and S. Lory. 2001. Identification of a genomic island present in the majority of pathogenic isolates of Pseudomonas aeruginosa. J. Bacteriol. 183:843-853.
Liu S, Yahr T L, Frank D W & Barbieri J T (1997) Biochemical relationships between the 53-kilodalton (Exo53) and 49-kilodalton (ExoS) forms of exoenzyme S of Pseudomonas aeruginosa. J. Bacteriol. 179: 1609-1613.
Logsdon, L. K., and J. Mecsas. 2003. Requirement of the Yersinia pseudotuberculosis effectors YopH and YopE in colonization and persistence in intestinal and lymph tissues. Infect. Immun. 71:4595-4607.
Lukashin, A. V., and M. Borodovsky. 1998. GeneMark.hmm: new solutions for gene finding. Nucleic Acids Res. 26:1107-1115.
Mahajan-Miklos, S., M.-W. Tan, L. G. Rahme, and F. M. Ausubel. 1999. Molecular mechanisms of bacterial virulence elucidated using a Pseudomonas aeruginosa-Caenorhabditis elegans pathogenesis model. Cell 96:47-56.
Mathee K, Narasimhan G, Valdes C et al. (2008) Dynamics of Pseudomonas aeruginosa genome evolution. P Natl Acad Sci USA 105: 3100-3105.
McKay D B & Steitz T A (1981) Structure of catabolite gene activator protein at 2.9 A resolution suggests binding to left-handed B-DNA. Nature 290: 744-749.
Mena J & Chen C (2007) Identification of strain-specific DNA of Actinobacillus actinomycetemcomitans by representational difference analysis. Oral Microbiol. Immunol. 22: 429-432.
Metcalf W W & Wolfe R S (1998) Molecular genetic analysis of phosphite and hypophosphite oxidation by Pseudomonas stutzeri WM88. J Bacteriol 180: 5547-5558.
Meyer, F., A. Goesmann, A. C. McHardy, D. Bartels, T. Bekel, J. Clausen, J. Kalinowski, B. Linke, O. Rupp, R. Giegerich, and A. Puhler. 2003. GenDB—an open source genome annotation system for prokaryote genomes. Nucleic Acids Res. 31:2187-2195.
Mitchell R E & Bieleski R L (1977) Involvement of Phaseolotoxin in Halo Blight of Beans: Transport and Conversion to Functional Toxin. Plant Physiol 60: 723-729.
Mougous J D, Cuff M E, Raunser S, et al. (2006) A virulence locus of Pseudomonas aeruginosa encodes a protein secretion apparatus. Science 312: 1526-1530.
Nagai H & Roy C R (2001) The DotA protein from Legionella pneumophila is secreted by a novel process that requires the Dot/Icm transporter. Embo J 20: 5962-5970.
Nakayama, K., S. Kanaya, M. Ohnishi, Y. Terawaki, and T. Hayashi. 1999. The complete nucleotide sequence of phi CTX, a cytotoxin-converting phage of Pseudomonas aeruginosa: implications for phage evolution and horizontal gene transfer via bacteriophages. Mol. Microbiol. 31:399-419.
Nakayama K, Takashima K, Ishihara H et al. (2000) The R-type pyocin of Pseudomonas aeruginosa is related to P2 phage, and the F-type is related to lambda phage. Mol Microbiol 38: 213-231.
Nicas, T. I., and B. H. Iglewski. 1984. Isolation and characterization of transposon-induced mutants of Pseudomonas aeruginosa deficient in production of exoenzyme S. Infect. Immun. 45:470-474.
Petersen L, Bollback J P, Dimmic M, Hubisz M & Nielsen R (2007) Genes under positive selection in Escherichia coli. Genome Res. 17: 1336-1343.
Qiu, X., A. U. Gurkar, and S. Lory. 2006. Interstrain transfer of the large pathogenicity island (PAPI-1) of Pseudomonas aeruginosa. Proc. Natl. Acad. Sci. USA 103:19830-19835.
Rahme, L. G., F. M. Ausubel, H. Cao, E. Drenkard, B. C. Goumnerov, G. W. Lau, S. Mahajan-Miklos, J. Plotnikova, M. W. Tan, J. Tsongalis, C. L. Walendziewicz, and R. G. Tompkins. 2000. Plants and animals share functionally common bacterial virulence factors. Proc. Natl. Acad. Sci. USA 97:8815-8821.
Rahme, L. G., E. J. Stevens, S. F. Wolfort, J. Shao, R. G. Tompkins, and F. M. Ausubel. 1995. Common virulence factors for bacterial pathogenicity in plants and animals. Science 268:1899-1902.
Rahme, L. G., M. W. Tan, L. Le, S. M. Wong, R. G. Tompkins, S. B. Calderwood, and F. M. Ausubel. 1997. Use of model plant hosts to identify Pseudomonas aeruginosa virulence factors. Proc. Natl. Acad. Sci. USA 94:13245-13250.
Ramos, J. L., M. Mailinez-Bueno, A. J. Molina-Henares, W. Teran, K. Watanabe, X. Zhang, M. T. Gallegos, R. Brennan, and R. Tobes. 2005. The TetR family of transcriptional repressors. Microbiol. Mol. Biol. Rev. 69:326-356.
Raymond, C. K., E. H. Sims, A. Kas, D. H. Spencer, T. V. Kutyavin, R. G. Ivey, Y. Zhou, R. Kaul, J. B. Clendenning, and M. V. Olson. 2002. Genetic variation at the O-antigen biosynthetic locus in Pseudomonas aeruginosa. J. Bacteriol. 184:3614-3622.
Reiter, W. D., P. Palm, and S. Yeats. 1989. Transfer RNA genes frequently serve as integration sites for prokaryotic genetic elements. Nucleic Acids Res. 17:1907-1914.
Rhame, F. S. 1979. The ecology and epidemiology of Pseudomonas aeruginosa, p. 31-51. In L. D. Sabath (ed.), Pseudomonas aeruginosa: the organism, diseases it causes, and their treatment. Hans Huber Publishers, Bern, Switzerland.
Riley M A & Wertz J E (2002) Bacteriocins: evolution, ecology, and application. Annu Rev Microbiol 56: 117-137.
Roy C R & Isberg R R (1997) Topology of Legionella pneumophila DotA: an inner membrane protein required for replication in macrophages. Infect Immun 65: 571-578.
Roy-Burman, A., R. H. Savel, S. Racine, B. L. Swanson, N. S. Revadigar, J. Fujimoto, T. Sawa, D. W. Frank, and J. P. Wiener-Kronish. 2001. Type III protein secretion is associated with death in lower respiratory and systemic Pseudomonas aeruginosa infections. J. Infect. Dis. 183:1767-1.774.
Sambrook, J., E. F. Fritsch, and T. Maniatis. 1989. Molecular cloning: a laboratory manual, 2nd ed. Cold Spring Harbor Laboratory Press, Cold Spring Harbor, N.Y.
Sastry, P. A., B. B. Finlay, B. L. Pasloske, W. Paranchych, J. R. Pearlstone, and L. B. Smillie. 1985. Comparative studies of the amino acid and nucleotide sequences of pilin derived from Pseudomonas aeruginosa PAK and PAO. J. Bacteriol. 164:571-577.
Schmidt K D, Tummler B & Romling U (1996) Comparative genome mapping of Pseudomonas aeruginosa PAO with P. aeruginosa C, which belongs to a major clone in cystic fibrosis patients and aquatic habitats. J Bacteriol 178: 85-93.
Schulert, G. S., H. Feltman, S. D. P. Rabin, C. G. Martin, S. E. Battle, J. Rello, and A. R. Hauser. 2003. Secretion of the toxin ExoU is a marker for highly virulent Pseudomonas aeruginosa isolates obtained from patients with hospital-acquired pneumonia. J. Infect. Dis. 188:1695-1706.
Schwartz E, Henie A, Cramm R, Eitinger T, Friedrich B & Gottschalk G (2003) Complete nucleotide sequence of pHG1: a Ralstonia eutropha H16 megaplasmid encoding key enzymes of H(2)-based ithoautotrophy and anaerobiosis. J Mol Biol 332: 369-383.
Schweizer, H. P., and T. T. Hoang. 1995. An improved system for gene replacement and xylE fusion analysis in Pseudomonas aeruginosa. Gene 158:15-22.
Sheahan K L, Cordero C L & Satchell K J (2004) Identification of a domain within the multifunctional Vibrio cholerae RTX toxin that covalently cross-links actin. Proc Natl Acad Sci USA 101: 9798-9803.
Shen K, Sayeed S, Antalis P et al. (2006) Extensive genomic plasticity in Pseudomonas aeruginosa revealed by identification and distribution studies of novel genes among clinical isolates. Infect Immun 74: 5272-5283.
Shinomiya T & Ina S (1989) Genetic comparison of bacteriophage PS17 and Pseudomonas aeruginosa R-type pyocin. J Bacteriol 171: 2287-2292.
Simon, R., U. Priefer, and A. Puhler. 1983. A broad host range mobilization system for in vivo genetic engineering: transposon mutagenesis in gram negative bacteria. Bio/Technology 1:784-791.
Spangenberg, C., R. Fislage, U. Roniling, and B. Tummler. 1997. Disrespectful type IV pilins. Mol. Microbiol. 25:203-204.
Spencer D H, Kas A, Smith E E et al. (2003) Whole-genome sequence variation among multiple isolates of Pseudomonas aeruginosa. J Bacteriol 185: 1316-1325.
Stover, C. K., X. Q. Pham, A. L. Erwin, S. D. Mizoguchi, P. Warrener, M. J. Hickey, F. S. L. Brinkman, W. O. Hufnagle, D. J. Kowalk, M. Lagrou, R. L. Garber, L. Goltry, E. Tolentino, S. Westbrock-Wadman, Y. Yuan, L. L. Brody, S. N. Coulter, K. R. Folger, A. Kas, K. Larbig, R. Lim, K. Smith, D. Spencer, G. K.-S. Wong, Z. Wu, I. T. Paulsen, J. Reizer, M. H. Saler, R. E. W. Hancock, S. Lory, and M. V. Olson. 2000. Complete genome sequence of Pseudomonas aeruginosa PAO1, an opportunistic pathogen. Nature 406:959-964.
Stryjewski, M. E., and D. J. Sexton. 2003. Pseudomonas aeruginosa infections in specific types of patients and clinical settings, p. 1-15. In A. R. Hauser and J. Rello (ed.), Severe infections caused by Pseudomonas aeruginosa, vol. 7. Kluwer Academic Publishers, Boston, Mass.
Vetting M W, Hegde S S, Fajardo J E, Fiser A, Roderick S L, Takiff H E & Blanchard J S (2006) Pentapeptide repeat proteins. Biochemistry 45: 1-10.
Vogel, H. J., and D. M. Bonner. 1956. Acetylornithinase of Escherichia coli: partial purification and some properties. J. Biol. Chem. 218:97-106.
Walker J E, Saraste M, Runswick M J & Gay N J (1982) Distantly related sequences in the alpha- and beta-subunits of ATP synthase, myosin, kinases and other ATP-requiring enzymes and a common nucleotide binding fold. Embo J 1: 945-951.
Wang Y D, Zhao S & Hill C W (1998) Rhs elements comprise three subfamilies which diverged prior to acquisition by Escherichia coli. J. Bacteriol. 180: 4102-4110.
White A K & Metcalf W W (2004) The htx and ptx operons of Pseudomonas Stutzeri WM88 are new members of the pho regulon. J Bacteriol 186: 5876-5882.
Wilderman P J, Vasil A I, Johnson Z & Vasil M L (2001) Genetic and biochemical analyses of a eukaryotic-like phospholipase D of Pseudomonas aeruginosa suggest horizontal acquisition and a role for persistence in a chronic pulmonary infection model. Mol. Microbiol. 39: 291-303.
Williams, K. P. 2002. Integration sites for genetic elements in prokaryotic tRNA and tmRNA genes: sublocation preference of integrase subfamilies. Nucleic Acids Res. 30:866-875.
Wolfgang, M. C., B. R. Kulasekara, X. Liang, D. Boyd, K. Wu, Q. Yang, C. G. Miyada, and S. Lory. 2003. Conservation of genome content and virulence determinants among clinical and environmental isolates of Pseudomonas aeruginosa. Proc. Natl. Acad. Sci. USA 100:8484-8489.
Woods, D. E., J. S. Lam, W. Paranchych, D. P. Speert, M. Campbell, and A. J. Godfrey. 1997. Correlation of Pseudomonas aeruginosa virulence factors from clinical and environmental isolates with pathogenicity in the neutropenic mouse. Can. J. Microbiol. 43:541-551.
Wurdemann, D., and B. Tummler. 2007. In silico comparison of pKLC102-like genomic islands of Pseudomonas aeruginosa. FEMS Microbiol. Lett. 275:244-249.
Yan Y, Yang J, Dou Y, et al. (2008) Nitrogen fixation island and rhizosphere competence traits in the genome of root-associated Pseudomonas stutzeri A1501. Proc. Natl. Acad. Sci. USA 105: 7564-7569.

In the foregoing description, certain terms have been used for brevity, clearness, and understanding. No unnecessary limitations are to be implied therefrom beyond the requirement of the prior art because such terms are used for descriptive purposes and are intended to be broadly construed. The different compositions and method steps described herein may be used alone or in combination with other compositions and method steps. It is to be expected that various equivalents, alternatives and modifications are possible. Citations to a number of non-patent references are made herein. The cited references are incorporated by reference herein in their entireties. In the event that there is an inconsistency between a definition of a term in the specification as compared to a definition of the term in a cited reference, the term should be interpreted based on the definition in the specification.

### Tables

TABLE 1
Characteristics of the 21 distinct subtractive hybridization
products with similarity to known sequences

| | No. of | |
|---|---|---|
| Region | clones | % Identity^{a} |
| Serotype 01 O-antigen | 12 | 95-99 |
| biosynthesis cluster | | |
| pilA gene | 1 | 99 |
| Phage φCTX DNA | 1 | 89^{b} |
| PAPI-1 | 1 | 95 |
| Rhs element | 2 | 91 (AA) |
| Site-specific recombinase | 2 | 37 (AA) |
| Phage-related protein | 1 | 33 (AA) |
| Zinc-binding transcriptional | 1 | 31 (AA) |
| regulator | | |

^{a}Nucleotide sequence identity, unless indicated as amino acid sequence identity (AA).
^{b}The sequence was 701 bp long and showed 89% similarity to φCTX in a 100-bp region but no significant similarity in the remaining sequence.

TABLE 2
ORFs of PAGI-5.

| | | | | | | | Homolog | | | | |
|---|---|---|---|---|---|---|---|---|---|---|---|
| | | | | | AA | GC | Accession | Gene | E-value | | Conserved |
| ORF | Annotation result | Orient | Start | Stop | length | % | number | name | (% identity) | | domain |
| 5PG1 | Putative integrase | − | 1442 | 162 | 426 | 60.7 | ABJ10138 | xerC | 0.0 | (98) | cd01182 |
| 5PG2 | Conserved hypothetical protein | − | 3358 | 1439 | 639 | 59.2 | AAP22591 | | 0.0 | (98) | pfam07514 |
| 5PG3 | Hypothetical protein | − | 3705 | 3607 | 32 | 60.6 | None | | | | None |
| 5PG4 | Possible methyltransferase | − | 5139 | 3877 | 420 | 53.4 | ABF09701 | | 5 × 10−100 | (46) | pfam01555 |
| 5PG5 | Hypothetical protein | + | 5620 | 7653 | 677 | 48.9 | AAX68258 | | 5 × 10−67 | (30) | None |
| 5PG6 | Hypothetical protein | − | 8260 | 7949 | 103 | 50.0 | None | | | | None |
| 5PG7 | Hypothetical protein | + | 8336 | 9391 | 351 | 49.6 | None | | | | None |
| 5PG8 | Plasmid stabilization system | − | 10160 | 9810 | 116 | 60.7 | EAZ62002 | parE | 3 × 10−61 | (100) | COG3668 |
| | protein | | | | | | | | | | |
| 5PG9 | Transcriptional regulator protein | − | 10436 | 10164 | 90 | 60.8 | AAP22603 | nikR | 3 × 10−44 | (100) | COG3609 |
| 5PG10 | Hypothetical protein similar to | + | 10620 | 10883 | 87 | 57.2 | AAG04531 | | 1 × 10−42 | (98) | pfam01527 |
| | PA0986 | | | | | | | | | | |
| 5PG11 | Transposase and inactivated | + | 11113 | 11766 | 217 | 60.2 | EAZ53156 | | 3 × 10−126 | (98) | pfam00655 |
| | derivatives | | | | | | | | | | |
| 5PG12 | Hypothetical protein | + | 11803 | 12156 | 117 | 50.3 | ABJ13911 | | 2 × 10−20 | (59) | None |
| 5PG13 | Conserved hypothetical protein | − | 13721 | 12186 | 511 | 59.2 | AAP22582 | | 0.0 | (96) | None |
| 5PG14 | Hypothetical protein | − | 14065 | 13718 | 115 | 62.9 | AAP94685 | | 8 × 10−58 | (100) | None |
| 5PG15 | Putative integral membrane protein | − | 15447 | 14065 | 460 | 64.6 | EAZ62006 | | 0.0 | (99) | None |
| 5PG16 | Conserved hypothetical protein | − | 16409 | 15471 | 312 | 65.8 | AAP94687 | | 0.0 | (100) | pfam07513 |
| 5PG17 | Putative integral membrane protein | − | 16840 | 16409 | 143 | 62.5 | AAP94688 | | 2 × 10−74 | (100) | pfam07511 |
| 5PG18 | hypothetical protein | + | 18978 | 19196 | 72 | 55.3 | EAZ62011 | | 3 × 10−33 | (100) | None |
| 5PG19 | Hypothetical outer membrane | − | 19852 | 19193 | 219 | 63.0 | EAZ62012 | dsbG | 8 × 10−124 | (100) | cd03023 |
| | protein | | | | | | | | | | |
| 5PG20 | Hypothetical protein | − | 20133 | 19849 | 94 | 58.2 | EAZ62013 | | 7 × 10−44 | (100) | None |
| 5PG21 | Type IV, secretory pathway, | − | 22934 | 20130 | 934 | 63.5 | ABR82757 | | 0.0 | (100) | COG3451 |
| | VirB4 components | | | | | | | | | | |
| 5PG22 | Conserved hypothetical protein | − | 23419 | 23072 | 115 | 64.1 | EAZ62014 | | 6 × 10−63 | (100) | None |
| 5PG23 | Conserved hypothetical protein | − | 24998 | 23493 | 501 | 64.1 | EAZ62015 | | 0.0 | (99) | None |
| 5PG24 | Conserved hypothetical protein | − | 25866 | 24982 | 294 | 66.1 | EAZ56168 | | 1 × 10−164 | (99) | None |
| 5PG25 | Hypothetical protein | − | 26522 | 25863 | 219 | 61.1 | EAZ62017 | | 7 × 10−128 | (100) | None |
| 5PG26 | Hypothetical protein | − | 26905 | 26519 | 128 | 66.4 | EAZ16018 | | 1 × 10−66 | (100) | None |
| 5PG27 | Hypothetical protein | − | 27272 | 26916 | 118 | 58.1 | EAZ62019 | | 6 × 10−61 | (100) | None |
| 5PG28 | Conserved hypothetical protein | − | 27529 | 27290 | 79 | 63.3 | ABJ3895 | | 6 × 10−34 | (94) | None |
| 5PG29 | Putative candidate type III effector | − | 27864 | 27526 | 112 | 66.4 | EAZ62021 | | 6 × 10−56 | (96) | pfam09686 |
| | Hop protein | | | | | | | | | | |
| 5PG30 | Aconitase H | − | 28256 | 27957 | 99 | 58.0 | ABD94651 | | 3 × 10−52 | (97) | None |
| 5PG31 | Putative pathogeuesis-related | − | 29894 | 28785 | 369 | 46.8 | EAZ62024 | | 0.0 | (99) | None |
| | protein | | | | | | | | | | |
| 5PG32 | Putative helicase | − | 31507 | 30026 | 493 | 59.8 | AAP22562 | | 0.0 | (98) | pfam00580 |
| 5PG33 | Cytochrome c biogenesis factor | − | 32264 | 31518 | 248 | 61.2 | ABD94655 | | 1 × 10−139 | (98) | None |
| 5GP34 | TraG/TraD family protein protein | − | 34495 | 32264 | 743 | 64.7 | EAZ56177 | | 0.0 | (99) | pfam02534 |
| 5PG35 | dTDP-D-glucose 4,6-dehydrase | − | 34768 | 34499 | 89 | 61.5 | ABR85411 | | 7 × 10−44 | (100) | None |
| 5PG36 | Conserved hypothetical protein | − | 35227 | 34777 | 166 | 65.5 | ABR84905 | | 6 × 10−89 | (100) | None |
| 5PG37 | Soluble lytic murein | − | 35855 | 35274 | 193 | 64.8 | ABJ13877 | | 2 × 10−106 | (99) | None |
| | transglycosylase | | | | | | | | | | |
| 5PG38 | Hypothetical protein | − | 36595 | 35840 | 251 | 65.5 | ABR86054 | | 1 × 10−132 | (97) | None |
| 5PG39 | Conserved hypothetical protein | − | 37295 | 36606 | 229 | 63.5 | EAZ56182 | | 1 × 10−127 | (99) | None |
| 5PG40 | Hypothetical protein | + | 37513 | 37785 | 90 | 56.0 | ABR13381 | | 6 × 10−45 | (100) | None |
| 5PG41 | Hypothetical protein | + | 38352 | 38489 | 45 | 59.4 | None | | | | None |
| 5PG42 | Hypothetical protein | + | 38591 | 38749 | 52 | 43.4 | None | | | | None |
| 5PG43 | Hypothetical protein | − | 39346 | 38954 | 130 | 46.1 | ABH09854 | | 3 × 10−17 | (42) | None |
| 5PG44 | Hypothetical protein | − | 39901 | 39698 | 67 | 56.9 | EAZ56183 | | 1 × 10−29 | (95) | None |
| 5PG45 | Hypothetical protein | − | 40494 | 39898 | 198 | 60.5 | EAZ56184 | | 4 × 10−100 | (91) | cd00198 |
| 5PG46 | Hypothetical protein | + | 40887 | 41069 | 60 | 62.8 | None | | | | None |
| 5PG47 | Hypothetical protein | − | 41462 | 41088 | 124 | 57.3 | None | | | | None |
| 5PG48 | Hypothetical protein | − | 43009 | 42326 | 227 | 61.3 | EAZ56186 | | 1 × 10−123 | (95) | pfam02586 |
| 5PG49 | SOS-response transcriptional | + | 43114 | 43545 | 143 | 61.8 | EAZ56187 | | 3 × 10−76 | (99) | COG1974 |
| | repressor | | | | | | | | | | |
| 5PG50 | Nucleotidyltransferase (fragment) | + | 43532 | 43672 | 46 | 58.9 | EAZ56188 | | 1 × 10−15 | (100) | cd01700 |
| 5PG51 | Site-specific recombinase, phage | + | 43770 | 44927 | 385 | 57.0 | AAZ35387 | | 3 × 10−97 | (82) | cd00798 |
| | integrase family | | | | | | | | | | |
| 5PG52 | Phage integrase | + | 44938 | 46554 | 537 | 48.8 | ABR80851 | | 0.0 | (99) | cd00798 |
| 5PG53 | Phage integrase like protein | + | 46544 | 48388 | 614 | 52.0 | YP_00134593 | | 0.0 | (100) | cd01182 |
| 5PG54 | TetR family transcriptional | + | 48385 | 48813 | 142 | 55.2 | AAP22553 | | 4 × 10−46 | (72) | None |
| | regulator | | | | | | | | | | |
| 5PG55 | Hypothetical protein | + | 49052 | 49234 | 60 | 61.2 | ABR84232 | | 4 × 10−26 | (100) | None |
| 5PG56 | MerR protein | − | 49787 | 49377 | 136 | 60.6 | ABR86667 | merR | 6 × 10−73 | (100) | cd1108 |
| 5PG57 | MerT protein | + | 49859 | 50209 | 116 | 61.0 | ABR82023 | merT | 5 × 10−59 | (100) | pfam02411 |
| 5PG58 | Periplasmic mereuic ion binding | + | 50223 | 50498 | 91 | 61.6 | ABR81298 | | 7 × 10−44 | (100) | cd00371 |
| | protein | | | | | | | | | | |
| 5PG59 | MerC | + | 50511 | 50945 | 144 | 63.7 | ABR83604 | merC | 1 × 10−75 | (100) | pfam03203 |
| 5PG60 | MerA | + | 50978 | 52660 | 560 | 65.2 | ABR83086 | merA | 0.0 | (100) | cd00371 |
| 5PG61 | Hypothetical protein | + | 52679 | 53095 | 138 | 64.5 | CAC80080 | | 3 × 10−45 | (78) | None |
| 5PG62 | Nucleotidyltransferase (fragment) | + | 53506 | 54660 | 384 | 61.8 | EAZ56188 | | 0.0 | (99) | cd01700 |
| 5PG63 | putative helicase | − | 57546 | 55297 | 749 | 63.6 | AAP22548 | | 0.0 | (99) | cd00079 |
| 5PG64 | Type I restriction-modification | − | 59097 | 57652 | 481 | 63.9 | AAP22547 | | 0.0 | (99) | COG0286 |
| | system methyltransferase | | | | | | | | | | |
| | subunit | | | | | | | | | | |
| 5PG65 | Hypothetical protein | − | 59732 | 59127 | 201 | 63.5 | AAP22600 | | 1 × 10−112 | (99) | None |
| 5PG66 | Conserved hypothetical protein | − | 60078 | 59824 | 84 | 61.6 | EAZ62038 | | 8 × 10−41 | (98) | None |
| 5PG67 | Conserved hypothetical protein | − | 60508 | 60146 | 120 | 58.7 | EAZ62039 | | 9 × 10−66 | (100) | None |
| 5PG68 | Conserved hypothetical protein | − | 61381 | 60611 | 256 | 61.5 | AAP22544 | | 2 × 10−127 | (93) | None |
| 5PG69 | Conserved hypothetical protein | − | 61788 | 61438 | 116 | 63.2 | ABR86730 | | 1 × 10−59 | (98) | None |
| 5PG70 | Conserved hypothetical protein | − | 62731 | 62024 | 235 | 53.0 | EAZ56198 | | 4 × 10−137 | (99) | None |
| 5PG71 | Hypothetical protein | − | 63123 | 62833 | 96 | 49.8 | EAZ56199 | | 7 × 10−48 | (94) | None |
| 5PG72 | Conserved hypothetical protein | − | 63449 | 63216 | 77 | 51.3 | EAZ62044 | | 4 × 10−36 | (94) | None |
| 5PG73 | Hypothetical protein | − | 63645 | 63487 | 52 | 56.6 | EAZ62045 | | 4 × 10−21 | (94) | None |
| SPG74 | Conerved hypothetical protein | − | 64270 | 63782 | 162 | 57.3 | ABJ13854 | | 9 × 10−90 | (99) | None |
| 5PG75 | Hypothetical protein | − | 64729 | 64595 | 44 | 52.6 | ABJ13853 | | 5 × 10−18 | (100) | None |
| 5PG76 | Hypothetical protein | − | 64907 | 64731 | 58 | 53.7 | AAP22537 | | 9 × 10−13 | (64) | None |
| 5PG77 | Hypothetical protein | − | 65371 | 64982 | 129 | 60.5 | EAZ62048 | | 3 × 10−66 | (97) | None |
| 5PG78 | Hypothetical protein | + | 65566 | 65742 | 58 | 61.6 | None | | | | None |
| 5PG79 | Hypothetical protein | + | 65901 | 65990 | 29 | 56.7 | EAZ56205 | | 2 × 10−17 | (96) | None |
| 5PG80 | PilM | − | 66723 | 66286 | 145 | 67.4 | AAP22535 | pilM | 7 × 10−75 | (98) | pfam07419 |
| 5PG81 | PilV | − | 68080 | 66752 | 442 | 63.7 | EAZ62050 | pilV | 0.0 | (99) | pfam04917 |
| 5PG82 | PilU | − | 69026 | 68085 | 313 | 65.5 | AAP22533 | pilU | 1 × 10−177 | (99) | cd01131 |
| 5PG83 | PilS | − | 69553 | 69023 | 176 | 60.1 | ZP_01363763 | pilS | 8 × 10−94 | (100) | pfam08805 |
| 5PG84 | PilR | − | 69673 | 69575 | 32 | 57.6 | ABJ13846 | pilR | 2 × 10−9 | (100) | None |
| 5PG85 | Type IV B pilus protein | − | 70656 | 69670 | 328 | 64.0 | ABJ13846 | pilR | 2 × 10−149 | (98) | pfam00482 |
| 5PG86 | PilQ2 | − | 71276 | 70656 | 206 | 64.9 | ABJ13845 | pilQ | 2 × 10−121 | (99) | cd01129 |
| 5PG87 | PilQ | − | 72237 | 71332 | 301 | 62.0 | AAP22531 | pilQ | 2 × 10−160 | (99) | cd01129 |
| 5PG88 | PilP | − | 72779 | 72246 | 177 | 71.2 | EAZ62055 | pilP | 8 × 10−93 | (98) | None |
| 5PG89 | PilO | − | 74094 | 72769 | 441 | 63.9 | ZP_01363767 | pilO | 0.0 | (99) | pfam06864 |
| 5PG90 | PilN | − | 75807 | 74098 | 569 | 63.8 | ABR83573 | pilN | 0.0 | (99) | pfam00263 |
| 5PG91 | PilL | − | 76931 | 75807 | 374 | 65.6 | AAP22527 | pilL | 0.0 | (95) | None |
| 5PG92 | Putative DNA helicase | − | 78992 | 77019 | 657 | 64.4 | ABJ13838 | | 0.0 | (98) | cd00269 |
| 5PG93 | Hypothetical protein | − | 80878 | 78989 | 629 | 60.8 | AAP22524 | | 0.0 | (94) | None |
| 5PG94 | Hypothetical protein | − | 81742 | 80990 | 250 | 57.5 | ABR83709 | | 5 × 10−146 | (100) | None |
| 5PG95 | Putative TopA topoisomerase | − | 83670 | 81751 | 639 | 61.4 | ABR81659 | | 0.0 | (99) | cd00186 |
| 5PG96 | Hypothetical protein | − | 84391 | 81197 | 64 | 55.4 | None | | | | None |
| 5PG97 | Single stranded DNA binding | − | 84983 | 84495 | 162 | 61.3 | EAZ62067 | ssb | 9 × 10−90 | (98) | cd04496 |
| | protein | | | | | | | | | | |
| 5PG98 | Conserved hypothetical protein | − | 85530 | 84997 | 177 | 60.9 | EAZ56223 | | 2 × 10−97 | (10) | None |
| 5PG99 | Conserved hypothetical protein | − | 86264 | 85536 | 242 | 62.7 | ABR84248 | | 2 × 10−139 | (100) | pfam08900 |
| 5PG100 | Hypothetical protein | − | 86618 | 86421 | 65 | 55.6 | ZP_01363778 | | 5 × 10−24 | (100) | None |
| 5PG101 | Hypothetical protein | − | 87205 | 86966 | 79 | 46.2 | ABJ13829 | | 1 × 10−37 | (96) | None |
| 5PG102 | Hypothetical protein | − | 87555 | 87196 | 119 | 62.8 | ABR86721 | | 1 × 10−21 | (69) | None |
| 5PG103 | Hypothetical protein | − | 86732 | 87806 | 308 | 59.0 | ABR85685 | | 7 × 10−156 | (97) | None |
| 5PG104 | Hypothetical protein | − | 88880 | 88698 | 60 | 56.8 | ABJ13827 | | 3 × 10−22 | (98) | None |
| 5PG105 | Hypothetical protein | − | 89644 | 88877 | 255 | 57.8 | ABJ13826 | | 6 × 10−142 | (97) | None |
| 5PG106 | Hypothetical protein | − | 89764 | 89672 | 30 | 62.4 | AAP22516 | | 2 × 10−19 | (100) | None |
| 5PG107 | Hypothetical protein | − | 91409 | 89775 | 544 | 58.9 | AAP22516 | | 0.0 | (95) | None |
| 5PG108 | Putative DNA binding protein | − | 91675 | 91406 | 89 | 59.6 | ABR81832 | | 1 × 10−40 | (95) | pfam03869 |
| 5PG109 | Hypothetical protein | − | 92157 | 91696 | 153 | 63.0 | ABJ13823 | | 1 × 10−41 | (98) | pfam04245 |
| 5PG110 | Hypothetical protein | − | 92390 | 92157 | 77 | 65.4 | ABJ13822 | | 7 × 10−18 | (100) | None |
| 5PG111 | Hypothetical protein | − | 92622 | 92383 | 79 | 64.6 | EAZ56231 | | 1 × 10−26 | (85) | None |
| 5PG112 | Hypothetical protein | − | 92872 | 92615 | 85 | 60.5 | EAZ62077 | | 1 × 10−44 | (100) | None |
| 5PG113 | Hypothetical protein | − | 93396 | 92869 | 175 | 60.6 | EAZ56233 | | 9 × 10−98 | (99) | None |
| 5PG114 | Hypothetical protein | − | 93571 | 93386 | 61 | 59.1 | None | | | | None |
| 5PG115 | Replicative DNA helicase | − | 94914 | 93568 | 448 | 60.8 | EAZ56234 | dnaB | 0.0 | (99) | cd00984 |
| 5PG116 | Hypothetical protein | − | 95663 | 94962 | 233 | 64.4 | EAZ56237 | | 2 × 10−133 | (100) | None |
| 5PG117 | Hypothetical protein | − | 96349 | 95663 | 228 | 64.5 | EAZ56238 | | 2 × 10−127 | (98) | None |
| 5PG118 | Hypothetical protein | − | 97056 | 96346 | 236 | 61.9 | AAP22503 | | 4 × 10−106 | (77) | None |
| 5PG119 | Hypothetical protein | − | 97559 | 97062 | 165 | 62.4 | AAP22502 | | 2 × 10−88 | (99) | None |
| 5PG120 | Hypothetical protein | − | 98293 | 97556 | 245 | 59.9 | EAZ56241 | | 7 × 10−140 | (100) | None |
| 5PG121 | Chromosome partitioning | − | 99161 | 98295 | 288 | 60.1 | EAZ56242 | [symbol] oj | 2 × 10−164 | (98) | COG1192 |
| | related protein | | | | | | | | | | |

[symbol]

indicates data missing or illegible when filed

TABLE 3
Primers used in screening the fosmid library

| Subtrac- | | | | |
|---|---|---|---|---|
| tive | | | | |
| hybridi- | | | | SEQ |
| zation | Sequence | Primer | | ID |
| product | similarity | name | Sequence | NO: |
| 2-14 | — | 2-14F | AGAATTTGACATGTTGCAGCG | 266 |
| | | 2-14R | AGCTTGCTCTCGGTCAATCTC | 267 |
| 2-53 | — | 2-53F | TACCCTATGACCATGCCCATT | 268 |
| | | 2-53R | TCAACCCCGAACAGCCTGA | 269 |
| 2-5 | PAPI-1 | 2-5F | ACTTGTAGACCAGGTGCGG | 270 |
| | | 2-5R | TGGATCATCACTGAGGCAGA | 271 |

TABLE 4
Characteristics of PSE9 genomic islands

| Genomic | Size | | Number of | | Insertion |
|---|---|---|---|---|---|
| island | (bp) | Location* | predicted ORFs | G + C % | site |
| PAGI-5^{†} | 99 276 | 1 061 197 | 121 | 59.6 | tRNA^{Lys} |
| PAGI-6 | 44 302 | 5 810 047 | 47 | 60.8 | tRNA^{Thr} |
| PAGI-7 | 22 479 | 4 439 857 | 20 | 55.8 | |
| PAGI-8 | 16 195 | 5 798 636 | 11 | 54.1 | tRNA^{Phe} |
| PAGI-9 | 7192 | 4 294 706 | 1 | 61.6 | |
| PAGI-10 | 2194 | 2 759 146 | 1 | 52.7 | |
| PAGI-11 | 2003 | 2 116 708 | 0 | 50.5 | |

*Genomic locations are given with respect to the PAO1 genome nomenclature (Stover et al., 2000).
^{†}The identification and characterization of PAGI-5 is presented elsewhere (Battle et al., 2008). It is included here for completeness.

TABLE 5
Distribution of PAGI sequences throughout the collection of PSE
clinical isolates

| Isolate | PAGI-6 | PAGI-7 | PAGI-8 | PAGI-9 | PAGI-10 |
|---|---|---|---|---|---|
| PSE1 | + | | | | + |
| PSE2 | | + | | + | |
| PSE3 | | + | | + | |
| PSE4 | | | | | + |
| PSE5 | | + | | + | |
| PSE6 | | + | | + | |
| PSE7 | | | | | + |
| PSE8 | | + | | + | |
| PSE9 | + | + | + | + | + |
| PSE10 | | | | | + |
| PSE11 | | | | | + |
| PSE12 | | + | | + | |
| PSE13 | | | | | + |
| PSE14 | | | | | + |
| PSE15 | | | | | + |
| PSE16 | | + | | + | |
| PSE17 | | | | | + |
| PSE18 | | | | | + |
| PSE19 | | + | | + | |
| PSE20 | | | | | + |
| PSE21 | | | | | + |
| PSE22 | | | | | + |
| PSE23 | | + | | + | |
| PSE24 | | | | | + |
| PSE25 | | + | | + | |
| PSE26 | | + | | + | |
| PSE27 | | + | | + | |
| PSE28 | | | | | + |
| PSE29 | | | | | + |
| PSE30 | | | | | + |
| PSE33 | | + | | + | |
| PSE35 | | | | | + |
| PSE37 | | + | | + | |
| PSE39 | | | | | + |
| PSE41 | | + | | + | |

TABLE 6
Annotation of ORFs in PAGI-6

| | | | | | | | Homolog | | | | |
|---|---|---|---|---|---|---|---|---|---|---|---|
| | | | | | AA | | Access. | Gene | | | Conserved |
| ORF | Annotation result | Orient | Start | Stop | length | GC % | Number | name | E-value (% identity) | | domain |
| 6PG1 | Predicted capsid packaging protein | − | 3342 | 2287 | 351 | 62.4 | ACD38654 | Q | 0.0 | (96) | COG5518 |
| 6PG2 | Predicted ATPase terminase | − | 5123 | 3342 | 593 | 63.1 | BAA36228 | P | 0.0 | (99) | COG5484 |
| | subunit | | | | | | | | | | pfam03237 |
| 6PG3 | Presumed capsid scaffold | + | 5258 | 6079 | 273 | 64.2 | BAA36229 | O | 2 × 10−133 | (99) | pfam05929 |
| 6PG4 | Predicted major capsid protein | + | 6115 | 7131 | 338 | 63.8 | BAA36230 | N | 2 × 10−172 | (99) | pfam05125 |
| 6PG5 | Predicted terminase subunit | + | 7137 | 7838 | 233 | 66.5 | ACD38658 | M | 1 × 10−130 | (100) | pfam05944 |
| 6PG6 | Predicted capsid completion | + | 7942 | 8403 | 153 | 66.2 | ACD38659 | L | 4 × 10−83 | (100) | pfam05926 |
| | protein | | | | | | | | | | |
| 6PG7 | Hypothetical protein | + | 8403 | 8615 | 70 | 69.5 | ACD38660 | X | 2 × 10−33 | (98) | pfam05489 |
| 6PG8 | Hypothetical protein | + | 8640 | 8993 | 117 | 68.6 | BAA36235 | | 3 × 10−19 | (100) | None |
| 6PG9 | Hypothetical protein | + | 8995 | 9267 | 90 | 67.4 | ACD38662 | | 2 × 10−41 | (100) | pfam05449 |
| 6PG10 | Hypothetical protein | + | 9264 | 10070 | 268 | 65.7 | BAA36237 | | 3 × 10−145 | (95) | pfam01471 |
| 6PG11 | Hypothetical protein | + | 10067 | 10309 | 80 | 60.1 | None | | | | None |
| 6PG12 | Predicted lysis | + | 10306 | 10767 | 153 | 68.4 | BAA36238 | lysB | 1 × 10−57 | (95) | None |
| 6PG13 | Predicted tail completion | + | 10845 | 11381 | 178 | 65.5 | ACD38665 | R | 5 × 10−98 | (99) | pfam06891 |
| 6PG14 | Predicted tail completion | + | 11374 | 11832 | 152 | 65.1 | BAA36241 | S | 2 × 10−73 | (91) | pfam05069 |
| 6PG15 | Hypothetical protein | + | 11842 | 12270 | 142 | 55.2 | ACD38666 | | 9 × 10−79 | (100) | None |
| 6PG16 | Predicted baseplate | + | 12448 | 13020 | 190 | 66.7 | ACD38667 | V | 4 × 10−102 | (98) | pfam04717 |
| 6PG17 | Predicted baseplate | + | 13017 | 13361 | 114 | 65.5 | ACD38668 | W | 1 × 10−59 | (99) | pfam04965 |
| 6PG18 | Predicted baseplate or tail fiber | − | 13358 | 14272 | 304 | 68.0 | BAA36245 | J | 1 × 10−171 | (99) | pfam04865 |
| | base | | | | | | | | | | |
| 6PG19 | Hypothetical protein | + | 14272 | 14808 | 178 | 67.6 | BAA36246 | I | 6 × 10−96 | (99) | COG4385 |
| 6PG20 | Hypothetical protein | + | 14810 | 17167 | 785 | 63.2 | BAA36247 | H | 0.0 | (87) | COG5301 |
| 6PG21 | Putative tail fiber assembly protein | + | 17164 | 17628 | 154 | 66.7 | BAA36248 | | 5 × 10−41 | (60) | None |
| 6PG22 | Putative tail sheath protein | + | 17719 | 18894 | 391 | 64.6 | BAA36249 | FI | 0.0 | (98) | pfam04984 |
| 6PG23 | Phage tail tube protein | + | 18951 | 19466 | 171 | 64.5 | ACD38674 | FII | 5 × 10−93 | (97) | pfam04985 |
| 6PG24 | Phage tail E | + | 19521 | 19850 | 109 | 65.8 | BAA36251 | E | 1 × 10−44 | (96) | pfam06158 |
| 6PG25 | Hypothetical protein | + | 19859 | 19978 | 39 | 65.0 | BAA36252 | E | 4 × 10−14 | (100) | None |
| 6PG26 | Phage related tail protein | + | 19968 | 22715 | 915 | 67.4 | ACD38677 | T | 0.0 | (95) | COG5283 |
| 6PG27 | Phage related tail protein | + | 22721 | 23161 | 146 | 68.0 | BAA36254 | U | 2 × 10−77 | (99) | pfam06995 |
| 6PG28 | Phage late control D | + | 23158 | 24444 | 428 | 67.9 | BAA36255 | D | 0.0 | (95) | pfam05954 |
| 6PG29 | Hypothetical protein | + | 25328 | 25861 | 177 | 44.9 | None | | | | None |
| 6PG30 | Hypothetical protein | − | 26265 | 25915 | 116 | 58.1 | BAA36258 | | 2 × 10−55 | (89) | None |
| 6PG31 | Hypothetical protein | − | 26434 | 26318 | 38 | 60.7 | BAA36259 | | 2 × 10−11 | (94) | None |
| 6PG32 | Hypothetical protein | + | 27266 | 27736 | 156 | 66.7 | BAA36261 | | 1 × 10−83 | (98) | None |
| 6PG33 | Hypothetical protein | + | 27733 | 28026 | 97 | 65.3 | BAA36262 | ogr | 4 × 10−50 | (97) | None |
| 6PG34 | Hypothetical protein | + | 28023 | 28373 | 116 | 63.5 | ACD38690 | | 8 × 10−60 | | None |
| 6PG35 | Hypothetical protein | + | 28444 | 28677 | 77 | 63.2 | BAA36264 | | 2 × 10−38 | (100) | None |
| 6PG36 | Hypothetical protein | + | 28674 | 31394 | 906 | 64.4 | BAA36265 | | 0.0 | (96) | cd1029 |
| 6PG37 | Hypothetical protein | + | 31439 | 31792 | 117 | 64.1 | ACD38693 | | 6 × 10−61 | (97) | None |
| 6PG38 | Hypothetical protein | + | 31804 | 32010 | 68 | 69.6 | BAA36267 | | 8 × 10−31 | (98) | pfam01258 |
| 6PG39 | Site-specific DNA methylase | + | 32314 | 33999 | 561 | 67.1 | BAA36269 | | 0.0 | (73) | COG0270 |
| | | | | | | | | | | | Pfam00145 |
| 6PG40 | Hypothetical protein | + | 34010 | 34198 | 62 | 55.6 | BAA36270 | | 1 × 10−6 | (76) | None |
| 6PG41 | Hypothetical protein | + | 34195 | 34407 | 70 | 59.2 | None | | | | None |
| 6PG42 | Site specific recombinase phage | + | 34404 | 35552 | 382 | 61.0 | AAZ34138 | | 1 × 10−163 | (70) | cd00800 |
| | integrase family | | | | | | | | | | |
| 6PG43 | Putative bacteriophage protein | + | 35794 | 36015 | 73 | 56.8 | AAO69527 | | 3 × 10−10 | (59) | None |
| 6PG44 | Prophage maint system killer | + | 36019 | 36525 | 168 | 57.0 | AAO69526 | | 2 × 10−43 | (58) | COG3654 |
| | protein | | | | | | | | | | |
| 6PG45 | Phage integrase | + | 37061 | 38380 | 439 | 60.8 | ACA75411 | | 0.0 | (98) | cd00801 |
| 6PG46 | Phage integrase | + | 39311 | 40291 | 326 | 59.2 | ACA75412 | | 3 × 10−161 | (86) | None |
| 6PG47 | Hypothetical protein | + | 42667 | 42765 | 32 | 33.7 | None | | | | None |

TABLE 7
Annotation of ORFs in PAGI-7

| | | | | | | | Homolog | | | | |
|---|---|---|---|---|---|---|---|---|---|---|---|
| | | | | | AA | | Access. | Gene | | | Conserved |
| ORF | Annotation result | Orient | Start | Stop | length | GC % | number | name | E-value (% identity) | | domain |
| 7PG1 | Site-specific recombinase | + | 115 | 1581 | 488 | 46.4 | ABP78291 | | 0.0 | (80) | cd01182 |
| 7PG2 | Hypothetical protein | − | 3320 | 3228 | 30 | 50.5 | None | | | | None |
| 7PG3 | Site-specific recombinase | + | 3725 | 5551 | 608 | 46.8 | ABP78293 | | 0.0 | (76) | None |
| 7PG4 | Predicted transcriptional regulator | − | 6336 | 6115 | 73 | 58.6 | BAF32907 | | 3 × 10−30 | (90) | cd00093 |
| 7PG5 | Conserved hypothetical protein | + | 6441 | 7019 | 192 | 57.5 | ACD39184 | | 5 × 10−98 | (92) | None |
| 7PG6 | Type III restriction enzyme | + | 7016 | 8416 | 466 | 57.4 | ACD39185 | | 1 × 10−117 | (50) | smart00487 |
| | | | | | | | | | | | smart00490 |
| 7PG7 | Hypothetical protein | + | 8507 | 8809 | 100 | 52.5 | ACD39186 | | 2 × 10−20 | (58) | None |
| 7PG8 | Hypothetical protein | − | 9036 | 8884 | 50 | 52.3 | None | | | | None |
| 7PG9 | Transposase | + | 9676 | 10011 | 111 | 61.6 | ACA75650 | | 7 × 10−51 | (90) | pfam05717 |
| 7GP10 | Transposase | − | 10415 | 10035 | 126 | 58.8 | BAB32744 | | 9 × 10−56 | (100) | None |
| 7GP11 | Transcriptional regulator (PtxE) | − | 11392 | 10523 | 289 | 62.5 | ABR80272 | ptxE | 6 × 10−163 | (98) | pfam03466 |
| 7GP12 | PtxD | − | 12396 | 11386 | 336 | 63.5 | AAC71709 | ptxD | 0.0 | (98) | COG1052 |
| 7GP13 | PtxC | − | 13232 | 12405 | 275 | 63.8 | ABR80270 | ptxC | 3 × 10−153 | (99) | COG3639 |
| 7GP14 | PtxB | − | 14104 | 13241 | 287 | 57.1 | AAC71707 | ptxB | 3 × 10−163 | (98) | COG3221 |
| 7GP15 | PtxA | − | 14928 | 14101 | 275 | 62.1 | AAC71706 | ptxA | 7 × 10−154 | (100) | cd03256 |
| 7GP16 | Transposase | + | 15271 | 15912 | 213 | 59.2 | ABR80273 | | 2 × 10−97 | (100) | COG3039 |
| 7GP17 | Reverse transcriptase | + | 16301 | 17569 | 422 | 58.9 | ABP79384 | | 6 × 10−179 | (87) | COG3344 |
| 7GP18 | Hypothetical protein | − | 18474 | 18301 | 57 | 55.2 | None | | | | None |
| 7GP19 | Hypothetical protein | − | 21108 | 19564 | 514 | 57.2 | EDO21782 | | 3 × 10−175 | (59) | smart00044 |
| | | | | | | | | | | | COG2114 |
| 7GP20 | Hypothetical protein | − | 22127 | 21189 | 312 | 55.4 | AAK24667 | | 8 × 10−76 | (47) | cd00038 |
| | | | | | | | | | | | COG4271 |

TABLE 8
Annotation of ORFs in PAGI-8

| | | | | | | | Homolog | | | | |
|---|---|---|---|---|---|---|---|---|---|---|---|
| | | | | | AA | | Access. | Gene | | | Conserved |
| ORF | Annotation result | Orient | Start | Stop | length | GC % | number | name | E-value (% identity) | | domain |
| 8PG1 | Site-specific recombinase | + | 90 | 1211 | 373 | 58.3 | AAO54077 | x | 9 × 10−137 | (70) | cd00796 |
| 8PG2 | Predicted ATPase | + | 1523 | 3601 | 692 | 48.8 | EAY29456 | x | 2 × 10−155 | (40) | None |
| 8PG3 | Hypothetical protein | + | 3953 | 4345 | 130 | 60.6 | ABJ11402 | x | 9 × 10−8 | (30) | None |
| 8PG4 | Hypothetical protein | + | 4488 | 5150 | 220 | 48.9 | ACA70219 | x | 9 × 10−6 | (23) | None |
| 8PG5 | Phage related DNA-bidning protein | + | 5196 | 6371 | 400 | 45.6 | CAG19734 | x | 1 × 10−78 | (38) | cd00093 |
| | | | | | | | | | | | COG2856 |
| 8PG6 | Hypothetical protein | + | 6386 | 6676 | 96 | 48.1 | None | | | | None |
| 8PG7 | Phage integrase | − | 8020 | 6923 | 365 | 52.1 | ABM43726 | x | 2 × 10−29 | (29) | cd01182 |
| 8PG8 | Putative TraY/DotA like protein | − | 10828 | 8573 | 751 | 60.2 | ABF09700 | x | 0.0 | (69) | None |
| 8PG9 | Hypothetical protein | − | 12730 | 12278 | 150 | 46.1 | None | | | | None |
| 8PG10 | Transposase | + | 13279 | 13542 | 87 | 57.2 | AAG04375 | x | 1 × 10−42 | (98) | pfam01527 |
| 8PG11 | Transposase | + | 13575 | 14417 | 280 | 61.6 | EAZ53156 | x | 8 × 10−163 | (99) | pfam00665 |
| 8PG12 | Pentapeptide repeat | − | 16063 | 14444 | 539 | 54.9 | ABO91967 | x | 2 × 10−44 | (29) | COG1357 |

TABLE 9
Annotation of ORFs in PAGI-9 and PAGI-10

| | | | | | AA | | Homolog Access. | Gene | | Conserved |
|---|---|---|---|---|---|---|---|---|---|---|
| ORF | Annotation result | Orient | Start | Stop | length | GC % | number | name | E-value (% identity) | domain |
| 9PG1 | Rhs family protein | − | 7192 | 521 | 2223 | 63.4 | EAZ59968 | x | 0.0 (96) | COG3209 |
| 10PG1 | Rhs family protein | + | 1 | 2457 | 818 | 67.4 | ABJ12620 | x | 0.0 (98) | COG3209 |

TABLE 10
Primers used in screening the fosmid library

| Sub. | | | | SEQ |
|---|---|---|---|---|
| Hyb. | Sequence | Primer | | ID |
| sequence | homology | name | Sequence | NO: |
| 2-3 | Site- | 2-3F | AAATTGGCCGAATACGCTT | 204 |
| | specific | 2-3R | TATTGCTTGCTGAATACCGGG | 205 |
| | recombi- | | | |
| | nase | | | |
| 2-30 | Site- | 2-30F | TCTCCAATCTTGAGTTGGGC | 206 |
| | specific | 2-30R | TTCTATCAACAGACCGGGATG | 207 |
| | recombi- | | | |
| | nase | | | |
| 2-7 | Zinc- | 2-7F | CAGCTGGACAAATTTATCG | 208 |
| | binding | 2-7R | AAATCCTACCCCACGGTGTAA | 209 |
| | transcrip- | | | |
| | tional | | | |
| | regulator | | | |
| 3-18 | Phage- | 3-18F | ACGCAAGTAGGGTCGTGAAAT | 210 |
| | related | 3-18R | TACTTTTTGAACCGTCGAGC | 211 |
| | protein | | | |
| 2-11 | — | 2-11F | ACTTTTTGAACCGTCGAGGTG | 212 |
| | | 2-11R | AACCAGGAGTTAACACGCAAG | 213 |
| 2-27 | — | 2-27F | AGGCGGTAGTCATGCGATG | 214 |
| | | 2-27R | TATCGCGGGGTGAATTTTTC | 215 |
| 3-23 | — | 3-23F | GCAGCCTGGACAAATTTATCG | 216 |
| | | 3-23R | ACCATCGAGATACACCATCCA | 217 |
| 2-13 | — | 2-13F | ATAAAGGATCTGCCCCAACG | 218 |
| | | 2-13R | TTAATCGAGTTGCTGAAGGC | 219 |
| 2-18 | — | 2-18F | CATTTAAAGAGGCGATCGATG | 220 |
| | | 2-18R | AATCCGACCTACTGCCTGAAC | 221 |
| 3-4 | — | 3-4F | TGATGGATCGATTGTATTGGG | 222 |
| | | 3-4R | GCCCTCAAGATCGGTAAAAT | 223 |
| 3-15 | — | 3-15F | ATTGAGAGTGGCAGAGGTTGA | 224 |
| | | 3-15R | ATGGCCCCTCTTTCGGTT | 225 |
| 2-32 | — | 2-32F | CATTTATTCGCTGTGGGACG | 226 |
| | | 2-32R | AATGTTAGGACCTCCTTGCG | 227 |
| 2-47 | — | 2-47F | CAGATGGAAGGAATCTCGGTCA | 228 |
| | | 2-47R | CGGGCAGGTACATAATAACG | 229 |
| 2-33 | — | 2-33F | AGTTTGAACACAGGAAAAGCG | 230 |
| | | 2-33R | GCCTAAAAGGACTACCGTCAG | 231 |
| 3-21 | — | 3-21F | GAACAGCTCGATTCTCATGCT | 232 |
| | | 3-21R | AATCTTGTCCGTTCGCAACA | 233 |
| 3-8 | Rhs | 3-8F | TTCCCAACTGCAGGAGGTAAA | 234 |
| | family | 3-8R | AAAATCAGCCATCACATCCC | 235 |
| | protein | | | |
| 2-2 | _{Φ}CTX DNA | 2-2F | TTGCAGAATCTTACCTGCAGC | 236 |
| | | 2-2R | AAAACACCAGCAGGACTACGA | 237 |
| 2-6 | — | 2-6F | ATGGAGTTCAAATGCATCGG | 238 |
| | | 2-6R | AATAATCTGCCCCCTCTTTCC | 239 |
| 2-41 | — | 2-41F | GCCGCGGGATTATGTTATATG | 240 |
| | | 2-41R | TATAAAGGATCTGCCCCAACG | 241 |
| 2-56 | Rhs | 2-56F | GATATACCCCCTATCTGAGCG | 242 |
| | family | 2-56R | TGTGGAATTGTGAGCGGATA | 243 |
| | protein | | | |
| 2-5 | PAPI-1 | 2-5F | ACTTGTAGACCAGGTGCGG | 244 |
| | | 2-5R | TGGATCATCACTGAGGCAGA | 245 |

TABLE 11
Primers used in screening clinical isolates for
PAGI sequences

| | | | SEQ | |
|---|---|---|---|---|
| Genomic | Primer | | ID | |
| Island | name | Sequence | NO: | RGP |
| PAGI-6 | PAGI6- | CGCGACTATAACCGGGCCAT | 246 | — |
| | 20250F | | | |
| | PAGI6- | CATTGCGCCGAGGATCTGCT | 247 | |
| | 20801R | | | |
| PAGI-7 | PAGI7- | CGGTCGATATTGCACGCCAG | 248 | — |
| | 6236F | | | |
| | PAGI7- | ATCGCTCTGCCTCGCCCATT | 249 | |
| | 6876R | | | |
| PAGI-8 | PAGI8- | TGCTGCTGTCGGGTATTACCTT | 250 | RGP6 |
| | 7050F | | | |
| | PAGI8- | CCCCTAACCGACATCCCTAACA | 251 | |
| | 8100R | | | |
| PAGI-9 | PAGI9- | TCGTAGCGGTAGCGAACGGA | 252 | — |
| | 2892F | | | |
| | PAGI9- | ACCAGCAAGGTCGACGCCAA | 253 | |
| | 3475R | | | |
| PAGI-10 | PAGI10- | GAACGCCTCGAATACGACGTC | 254 | RGP2 |
| | 472F | | | |
| | PAGI10- | TTGTCGCCTACTGCCAGGGT | 255 | |
| | 1292R | | | |
| PAGI-11 | Not | | | RGP5 |
| | screened | | | |

We claim:

1. A method for detecting a virulent strain of Pseudomonas bacteria in a sample, the method comprising detecting at least a fragment of PAGI-5, PAGI-6, PAGI-7, PAGI-8, PAGI-9, PAGI-10, or PAGI-11 nucleic acid in the sample, thereby detecting the virulent strain of Pseudomonas bacteria.

2. The method of claim 1, comprising:
(a) amplifying at least a fragment of PAGI-5, PAGI-6, PAGI-7, PAGI-8, PAGI-9, PAGI-10, or PAGI-11 from the sample to obtain amplified DNA; and
(b) detecting the amplified DNA, thereby detecting the virulent strain of Pseudomonas bacteria.

3. The method of claim 1, wherein the virulent strain of Pseudomonas bacteria is a virulent strain of Pseudomonas aeruginosa.

4. The method of claim 1, wherein the sample is a biological sample from a patient.

5. The method of claim 1, wherein the detected fragment comprises at least about 10 contiguous nucleotides of PAGI-5, PAGI-6, PAGI-7, PAGI-8, PAGI-9, PAGI-10, or PAGI-11.

6. The method of claim 1, wherein the detected fragment comprises at least about 10 contiguous nucleotides of PAGI-5 within novel region I (NR-I) or novel region II (NR-II).

7. The method of claim 1, wherein the detected fragment comprises at least about 10 contiguous nucleotides within an ORF present in PAGI-5, PAGI-6, PAGI-7, PAGI-8, PAGI-9, PAGI-10, or PAGI-11.

8. The method of claim 1, comprising:
(a) isolating nucleic acid from the sample;
(b) contacting the isolated nucleic with an oligonucleotide that specifically hybridizes to nucleic acid of PAGI-5, PAGI-6, PAGI-7, PAGI-8, PAGI-9, PAGI-10, or PAGI-11; and
(c) detecting hybridization of the oligonucleotide to the isolated nucleic acid, thereby detecting the virulent strain of Pseudomonas bacteria.

9. The method of claim 8, wherein the oligonucleotide comprises a label and detecting hybridization of the oligonucleotide to the isolated nucleic acid comprises detecting a signal from the label.

10. The method of claim 8, comprising contacting the isolated nucleic with a pair of oligonucleotides that function as primers and wherein detecting hybridization of the oligonucleotide to the isolated nucleic acid comprises amplifying at least a portion of the isolated nucleic acid.

11. The method of claim 8, further comprising amplifying at least a portion of the isolated nucleic acid.

12. The method of claim 1, further comprising detecting at least a fragment of PAPI-1 or PAPI-2 nucleic acid in the sample, thereby detecting the virulent strain of Pseudomonas bacteria.

13. The method of claim 12, wherein the detected PAPI-1 or PAPI-2 nucleic acid in the sample comprises exoU nucleic acid.

14. The method of claim 1, wherein the virulent strain of Pseudomonas bacteria has an LD50 in mice that is no more than about 1.3×10⁶ CFU.

15. A method for detecting a virulent strain of Pseudomonas bacteria in a sample, the method comprising:
(a) reacting the sample with an antibody that binds specifically to a polypeptide encoded by an ORF present in PAGI-5, PAGI-6, PAGI-7, PAGI-8, PAGI-9, PAGI-10, or PAGI-11; and
(b) detecting binding of the antibody to the polypeptide, thereby detecting the virulent strain of Pseudomonas bacteria in the sample.

16. The method of claim 15, wherein the virulent strain of Pseudomonas bacteria is a virulent strain of Pseudomonas aeruginosa.

17. The method of claim 15, wherein the sample is a biological sample from a patient.

18. The method of claim 15, wherein the detected polypeptide is encoded by an ORF present in PAGI-5 within novel region I (NR-I) or novel region II (NR-II).

19. The method of claim 15, wherein the antibody comprises a label and detecting binding of the antibody to the polypeptide comprises detecting a signal from the label.

20. The method of claim 15, further comprising reacting the sample with an antibody that binds specifically to a polypeptide encoded by an ORF present in PAPI-1 or PAPI-2.

21. The method of claim 15, wherein the virulent strain of Pseudomonas bacteria has an LD50 in mice that is no more than about 1.3×10⁶ CFU.
